SEQUENCE LISTING (6 sequences)

SEQ ID NO: 1 - DNA, 13 bp, synthetic construct
  source 1..13: mol_type = other DNA
tvvvvvtvvvvvt

SEQ ID NO: 2 - DNA, 12 bp, synthetic construct
  source 1..12: mol_type = other DNA
aaattgccccaa

SEQ ID NO: 3 - DNA, 10 bp, synthetic construct
  source 1..10: mol_type = other DNA
tttaacgggg

SEQ ID NO: 4 - DNA, 15 bp, synthetic construct
  source 1..15: mol_type = other DNA
bbbbabbbbabbbba

SEQ ID NO: 5 - DNA, 12 bp, synthetic construct
  source 1..12: mol_type = other DNA
abbbbbabbbba

SEQ ID NO: 6 - DNA, 13 bp, synthetic construct
  source 1..13: mol_type = other DNA
bbbabbabbbaba

(19) United States
(12) Patent Application Publication
(10) Pub. No.: US 2026/0218296 A1
(43) Pub. Date: Jul. 30, 2026

(54) SYSTEMS, METHODS, AND COMPOSITIONS FOR SEQUENCING

(71) Applicant: Ultima Genomics, Inc., Fremont, CA (US)

(72) Inventors: Gilad Almogy, Palo Alto, CA (US); Florian Oberstrass, Redwood City, CA (US); Charles Francavilla, Fremont, CA (US); Theo Nikiforov, Carlsbad, CA (US); Robert Ono, Palo Alto, CA (US); Derek Fleck, Fremont, CA (US)

(21) Appl. No.: 19/313,329

(22) Filed: Aug. 28, 2025

Related U.S. Application Data
(63) Continuation of application No. PCT/US2024/018563, filed on Mar. 5, 2024.
(60) Provisional application No. 63/450,205, filed on Mar. 6, 2023.
(60) Provisional application No. 63/450,618, filed on Mar. 7, 2023.
(60) Provisional application No. 63/488,969, filed on Mar. 7, 2023.
(60) Provisional application No. 63/581,542, filed on Sep. 8, 2023.

(51) Int. Cl. C12Q 1/6874; C12Q 1/6813

(52) U.S. Cl. CPC C12Q 1/6874; C12Q 1/6813

(57) ABSTRACT

Provided herein are systems, methods, and compositions for sequencing.

### CROSS-REFERENCE

This application is a continuation application of International Application No. PCT/US2024/18563 filed on Mar. 5, 2024, which claims the benefit of U.S. Provisional App. Nos. 63/450,205 filed on Mar. 6, 2023, 63/450,618 filed on Mar. 7, 2023, 63/488,969 filed on Mar. 7, 2023, and 63/581,542 filed on Sep. 8, 2023, each of which is entirely incorporated by reference herein for all purposes.

### REFERENCE TO AN ELECTRONIC SEQUENCE LISTING

This application contains a Sequence Listing, which has been submitted electronically in xml format and is hereby incorporated by reference in its entirety. Said xml copy, created on Jan. 6, 2026, is named SeqList-368893-41001.xml and is 6,272 bytes in size.

### BACKGROUND

Biological sample processing has various applications in the fields of molecular biology and medicine (e.g., diagnosis). For example, nucleic acid sequencing may provide information that may be used to diagnose a certain condition in a subject and in some cases tailor a treatment plan. Sequencing is widely used for molecular biology applications, including vector designs, gene therapy, vaccine design, industrial strain design and verification. Biological sample processing may involve a fluidics system and/or a detection system.

Nucleic acid sequencing may comprise the use of fluorescently labeled moieties. Such moieties may be labeled with organic fluorescent dyes. The sensitivity of a detection scheme can be improved by using dyes with both a high extinction coefficient and quantum yield, where the product of these characteristics may be termed the dye's “brightness.” Dye brightness may be attenuated by quenching phenomena, including quenching by biological materials, quenching by proximity to other dyes, and quenching by solvent. Other routes to brightness loss include photobleaching, reactivity to molecular oxygen, and chemical decomposition.

### SUMMARY

DNA origami is a revolutionary and innovative technique in the field of nanotechnology that harnesses the unique properties of DNA molecules to create intricate and programmable nanostructures. This method involves the design and self-assembly of DNA strands into specific shapes and patterns, mimicking the art of origami but at a microscopic scale.

The process of DNA origami typically begins with a long, single-stranded DNA scaffold, which serves as the backbone for the desired structure. Shorter DNA strands, known as staple strands, are then designed to complement specific regions of the scaffold, guiding it into the desired shape through Watson-Crick base pairing. The combination of these carefully designed staple strands and the scaffold results in the formation of intricate and precisely defined nanoscale structures.

What is disclosed are methods to attach DNA molecules, short or long, to a substrate via DNA origami scaffold. Also disclosed are compositions and process for achieving the same.

Provided herein are systems and methods that use DNA nanostructures as supports to prepare for use in amplification and/or sequencing. Further, the present disclosure provides labeled (e.g., detectable) reagents and the use of these reagents in nucleic acid processing (e.g., sequencing). The methods and materials provided herein may reduce fluorescent quenching in reagents with multiple labeling moieties (e.g., fluorescent dyes). Quenching can reduce the precision at which labeling moieties are detected during nucleic acid processing, and hence can negatively impact nucleic acid sequencing quality and downstream analysis. The present disclosure recognizes the need for methods and materials for improved labeled reagents.

Further provided herein are systems and methods for improving accuracy of sequencing homopolymer regions. Sequencing homopolymeric regions with labeled nucleotides presents a wide range of challenges. For example, sequencing with reversibly terminated nucleotides can be prohibitively slow, especially when sequencing large portions of a genome. Sequencing using non-terminated nucleotides and simultaneously detecting multiple adjacent labeled nucleotides can generate quenching interactions between detectable sequencing reagents (e.g., dye-coupled nucleotides). Cleaving labels from reagents can diminish quenching interactions but can also generate chemical scars for both terminated and non-terminated sequencing, which scars can inhibit detection, reagent activity, and nucleic acid polymerization. Therefore, new reagents and methods are needed for sequencing using labeled reagents, where cleavage of labels during the sequencing can generate chemical scars. The present disclosure may be advantageous to improve sequencing results.

In an aspect, provided is a labeled reagent comprising: an object; a linker, comprising a cleavable portion; a nucleic acid moiety, wherein the nucleic acid moiety is attached to the object via the linker; and one or more detectable moieties coupled to the nucleic acid moiety. In some embodiments, the object comprises a nucleotide base. In some embodiments, the object comprises a protein.

In some embodiments, the nucleic acid moiety comprises an oligonucleotide. In some embodiments, the oligonucleotide is double-stranded, comprising a first strand and a second strand. In some embodiments, the first strand of the oligonucleotide is coupled to the one or more detectable moieties. In some embodiments, the second strand of the oligonucleotide is not covalently coupled to the one or more detectable moieties.

In some embodiments, the first strand of the oligonucleotide comprises a sequence of at least a first and a second canonical base type, wherein bases of the first canonical base type are coupled to detectable moieties. In some embodiments, the sequence of the first strand of the oligonucleotide comprises an alternation of the first and second canonical base types, respectively. In some embodiments, the sequence of the first strand of the oligonucleotide comprises a series of nucleotide bases in the following order: one or more nucleotide bases of the second canonical base type (Z); and a nucleotide base of the first canonical base type (X).

In some embodiments, the sequence of the first strand of the oligonucleotide comprises a series of nucleotide bases of the first canonical base type (X) and second canonical base type (Z) in the form of (ZnX)i, wherein: n is a number of bases of the second canonical base type (Z), wherein n is an integer between 1 and 20; and i is a number of repeating units of a nucleotide base of the first canonical base type and n nucleotide bases of the second canonical base type, wherein i is an integer between 1 and 10.

In some embodiments, the first strand of the oligonucleotide comprises a sequence of at least three canonical base types. In some embodiments, the first strand of the oligonucleotide comprises a sequence of at least four canonical base types. In some embodiments, only a single canonical base type is coupled to detectable moieties.

In some embodiments, the nucleic acid moiety comprises a predetermined two dimensional or three-dimensional shape. In some embodiments, the predetermined two dimensional or three-dimensional shape encloses the one or more detectable moieties. In some embodiments the predetermined two dimensional or three-dimensional shape further comprises one or more attachment sites for coupling to detectable moieties.

In some embodiments, the predetermined two- or three-dimensional shape comprises one or more single stranded nucleic acid molecules. In some embodiments, the predetermined two- or three-dimensional shape comprises double stranded or partially double stranded nucleic acid molecules.

In some embodiments, the one or more detectable moieties coupled to the nucleic acid moiety comprise fluorescent dyes. In some embodiments, the fluorescent dyes comprise ATTO 390, ATTO 425, ATTO 465, ATTO 488, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 643, ATTO 647, ATTO 655, ATTO Oxa12, ATTO 665, ATTO 680, ATTO 700, ATTO 725, ATTO 740, KK1119, KK9046, Abberior STAR635, Abberior STAR635P, or Kam.

In some embodiments, the one or more detectable moieties coupled to the nucleic acid moiety comprise one or more fluorescent nanoparticles. In some embodiments, the one or more fluorescent nanoparticles comprise Q-dots. In some embodiments, the one or more fluorescent nanoparticles comprise fluorescent beads. In some embodiments, the one or more fluorescent nanoparticles comprise gel particles.

In another aspect, a method for sequencing, comprises providing a primer-hybridized template nucleic acid molecule; contacting the primer-hybridized template nucleic acid molecule with nucleotides, wherein at least a subset of the nucleotides comprises a labeled reagent according to embodiments described above.

In some embodiments, the method further comprises detecting one or more signals from the primer-hybridized template nucleic acid molecule. In some embodiments, the nucleotides are of a first canonical base type.

In another aspect, provided is a method of pre-enrichment, comprising: contacting a template nucleic acid to a support, wherein the support comprises a DNA nanostructure, the DNA nanostructure comprising (i) a pre-enrichment site configured to bind to the template nucleic acid and (ii) an amplification site configured to bind an amplified derivative of the template nucleic acid, to generate a support-template complex, wherein the DNA nanostructure comprises fewer pre-enrichment sites than amplification sites, wherein the template nucleic acid is capable of hybridizing to the pre-enrichment site and not to the amplification site.

In some embodiments, the pre-enrichment site comprises a first oligonucleotide molecule comprising (1) a first sequence comprising a capture sequence complementary to an adapter sequence of the template nucleic acid and (2) a second sequence comprising an amplification primer sequence, and wherein the amplification site comprises a second oligonucleotide molecule comprising the second sequence, wherein the amplification site does not comprise the first sequence. In some embodiments, the template nucleic acid is hybridized to the first sequence, and further comprising extending (1) the first oligonucleotide molecule to generate a first extended molecule and (2) the template nucleic acid to generate a second extended molecule. In some embodiments, the second extended molecule is removed from the first extended molecule, and the method further comprises attaching the second extended molecule or a derivative of the second extended molecule to the second oligonucleotide molecule.

In some embodiments, the DNA nanostructure comprises a plurality of amplification sites. In some embodiments, the DNA nanostructure comprises at most 1% pre-enrichment sites from all attachment sites including pre-enrichment sites and amplification sites on the DNA nanostructures. In some embodiments, the DNA nanostructure is bound to at most one template nucleic acid. In some embodiments, the DNA nanostructure further comprises a surface attachment site configured to attach to a binder of a substrate.

In some embodiments, the method further comprises contacting a plurality of template nucleic acids, including the template nucleic acid, and a plurality of supports, including the support, to generate a plurality of support-template complexes wherein a majority of the plurality of support-template complexes comprises a single template nucleic acid of the plurality of template nucleic acids. In some embodiments, the plurality of template nucleic acids is provided at lower concentration than the plurality of supports.

In some embodiments, the method further comprises providing a diffusion-limiting agent with the support and the template nucleic acid. In some embodiments, the diffusion-limiting agent comprises polyethylene glycol (PEG). In some embodiments, the method further comprises constructing the DNA nanostructure using a scaffold strand and a plurality of staple strands. In some embodiments, the DNA nanostructure comprises a cross-link. In some embodiments, the DNA nanostructure comprises a dideoxy NTP (ddNTP). In some embodiments, the method further comprises loading the support-template complex onto a substrate.

In an aspect, provided is a composition, comprising: a support, wherein the support comprises a DNA nanostructure, the DNA nanostructure comprising (i) a pre-enrichment site configured to bind to a template nucleic acid and (ii) an amplification site configured to bind an amplified derivative of the template nucleic acid, wherein the DNA nanostructure comprises fewer pre-enrichment sites than amplification sites, wherein the template nucleic acid is capable of hybridizing to the pre-enrichment site and not to the amplification site.

In some embodiments, the pre-enrichment site comprises a first oligonucleotide molecule comprising (1) a first sequence comprising a capture sequence complementary to an adapter sequence of the template nucleic acid and (2) a second sequence comprising an amplification primer sequence, and wherein the amplification site comprises a second oligonucleotide molecule comprising the second sequence, wherein the amplification site does not comprise the first sequence.

In some embodiments, the composition further comprises a plurality of supports, each support of the plurality of supports comprising a DNA nanostructure. In some embodiments, the DNA nanostructure further comprise a surface attachment site. In some embodiments, the DNA nanostructure comprises a cross-link. In some embodiments, the DNA nanostructure comprises a dideoxy NTP (ddNTP).

In some embodiments, the composition further comprises the template nucleic acid. In some embodiments, the template nucleic acid is not bound to the support. In some embodiments, the template nucleic acid is bound to the support. In some embodiments, the composition further comprises a substrate. In some embodiments, the composition further comprises a diffusion-limiting agent. In some embodiments, the diffusion-limiting agent comprises polyethylene glycol (PEG).

In another aspect, provided is a kit, comprising: a support, wherein the support comprises a DNA nanostructure, the DNA nanostructure comprising (i) a pre-enrichment site configured to bind to a template nucleic acid and (ii) an amplification site configured to bind an amplified derivative of the template nucleic acid, wherein the DNA nanostructure comprises fewer pre-enrichment sites than amplification sites, wherein the template nucleic acid is capable of hybridizing to the pre-enrichment site and not to the amplification site.

In some embodiments, the pre-enrichment site comprises a first oligonucleotide molecule comprising (1) a first sequence comprising a capture sequence complementary to an adapter sequence of the template nucleic acid and (2) a second sequence comprising an amplification primer sequence, and wherein the amplification site comprises a second oligonucleotide molecule comprising the second sequence, wherein the amplification site does not comprise the first sequence.

In some embodiments, the kit further comprises a plurality of supports, each support of the plurality of supports comprising a DNA nanostructure. In some embodiments, the DNA nanostructure further comprise a surface attachment site. In some embodiments, the DNA nanostructure comprises a cross-link. In some embodiments, the DNA nanostructure comprises a dideoxy NTP (ddNTP).

In some embodiments, the kit further comprises the template nucleic acid. In some embodiments, the template nucleic acid is not bound to the support. In some embodiments, the template nucleic acid is bound to the support. In some embodiments, the kit further comprises a substrate. In some embodiments, the kit further comprises a diffusion-limiting agent. In some embodiments, diffusion-limiting agent comprises polyethylene glycol (PEG).

In another aspect, provided is a method, comprising contacting a plurality of supports to a substrate to immobilize the plurality of supports to the substrate, wherein the plurality of supports comprises a plurality of DNA nanostructures, wherein the plurality of DNA nanostructures comprises amplification sites.

In another aspect, provided is a method for sequencing a nucleic acid molecule, comprising: (a) contacting a first nucleotide solution to a growing nucleic acid strand hybridized to the nucleic acid molecule, wherein the first nucleotide solution comprises first labeled nucleotides; (b) detecting a signal from the growing nucleic acid strand indicative of incorporation of a labeled nucleotide of the first labeled nucleotides; (c) contacting with the growing nucleic acid strand (i) a cleavage reagent configured to cleave a label from the labeled nucleotide to the growing nucleic acid strand and (ii) a capping reagent configured to generate a capped moiety on the growing nucleic acid strand from a cleaved linker of the labeled nucleotide; (d) contacting a second nucleotide solution to the growing nucleic acid strand, wherein the second nucleotide solution comprises second labeled nucleotides, wherein the first nucleotide solution and the second nucleotide solution comprise nucleotides of the same canonical base type; and (e) detecting a second signal from the growing nucleic acid strand indicative of incorporation of a labeled nucleotide of the second labeled nucleotides.

In some embodiments, the method further comprises using the first signal and the second signal to determine a sequencing read of the nucleic acid molecule.

In some embodiments, the capping reagent comprises a disulfide group. In some embodiments, the capping reagent comprises dipyridyl disulfide (DPDS) or pyridyl ethyl amine disulfide (PEAD). In some embodiments, the capping reagent is provided to the growing nucleic acid strand in a mixture with the second nucleotide solution.

In some embodiments, the first labeled nucleotides are non-terminated nucleotides. In some embodiments, the first labeled nucleotides and the second labeled nucleotides comprise a single canonical base type. In some embodiments, the first labeled nucleotides and the second labeled nucleotides comprise a same type of dye. In some embodiments, the first nucleotide solution comprises a mixture of labeled and unlabeled nucleotides.

In some embodiments, the nucleic acid molecule is immobilized to a substrate. In some embodiments, nucleic acid molecule is coupled to a bead immobilized to the substrate. In some embodiments, the bead comprises a plurality of nucleic acid molecules, including the nucleic acid molecule, comprising an identical sequence, wherein the plurality of nucleic acid molecules are hybridized to a plurality of growing nucleic acid strands, including the growing nucleic acid strand.

In some embodiments, in (c), cleaving of the label from the labeled nucleotide by the cleavage reagent generates a thiol scar on the growing nucleic acid strand.

In some embodiments, the cleavage reagent is selected from the group consisting of tris(3-hydroxypropyl) phosphine (THP), β-mercaptoethanol (β-ME), dithiothreitol (DTT), tris(2-carboxyethyl) phosphine (TCEP), Ellman's reagent, hydroxylamine, glutathione, and cyanoborohydride.

In some embodiments, the labeled nucleotide of the first labeled nucleotides comprises a cleavable linker, wherein the cleavable linker comprises a disulfide bond. In some embodiments, the labeled nucleotide of the first labeled nucleotides comprises a hydroxyproline linker.

In another aspect, provided is a method for sequencing a nucleic acid molecule, comprising: (a) incorporating a labeled nucleotide into a growing nucleic acid strand hybridized to a nucleic acid molecule, wherein the labeled nucleotide is coupled to a dye via a cleavable linker; (b) detecting a signal from the dye; (c) cleaving the cleavable linker; and (d) contacting the growing nucleic acid strand with a capping reagent, wherein the capping reagent comprises pyridyl ethyl amine disulfide.

In another aspect, provided is a kit for sequencing, comprising: a plurality of labeled nucleotides comprising a cleavable linker; and a capping reagent comprising pyridyl ethyl amine disulfide.

In some embodiments, the kit further comprises a cleavage reagent. In some embodiments, the cleavage reagent is selected from the group consisting of tris(3-hydroxypropyl) phosphine (THP), β-mercaptoethanol (β-ME), dithiothreitol (DTT), tris(2-carboxyethyl) phosphine (TCEP), Ellman's reagent, hydroxylamine, glutathione, and cyanoborohydride.

In another aspect, provided is a method for sequencing a nucleic acid molecule, comprising: (a) incorporating a labeled nucleotide into a growing nucleic acid strand hybridized to a nucleic acid molecule, wherein the labeled nucleotide is coupled to a dye via a cleavable linker; (b) detecting a signal from the dye; (c) cleaving the cleavable linker; and (d) contacting the growing nucleic acid strand with a capping reagent, wherein the capping reagent comprises pyridyl ethyl amine disulfide.

In another aspect, provided is a kit for sequencing, comprising: a plurality of labeled nucleotides comprising a cleavable linker; and a capping reagent comprising pyridyl ethyl amine disulfide.

In some embodiments, the kit further comprises a cleavage reagent. In some embodiments, the cleavage reagent is selected from the group consisting of tris(3-hydroxypropyl) phosphine (THP), β-mercaptoethanol (β-ME), dithiothreitol (DTT), tris(2-carboxyethyl)phosphine (TCEP), Ellman's reagent, hydroxylamine, glutathione, and cyanoborohydride.

In another aspect, a method is provided comprising: (a) contacting a growing strand hybridized to a template with a first reagent mixture comprising labeled, non-terminated bases and reversibly terminated bases of a first same canonical base type and detecting a first signal indicative of incorporation of at least a subset of the labeled, non-terminated bases of the first reagent mixture in the growing strand, or lack thereof, to generate first sequencing data; (b) reversing termination of the reversibly terminated bases of the first reagent mixture incorporated in the growing strand, if any; (c) contacting the growing strand with a second reagent mixture comprising labeled, non-terminated bases and terminated bases of the first same canonical base type and detecting a second signal indicative of incorporation of at least a subset of the labeled, non-terminated bases of the second reagent mixture in the growing strand, or lack thereof, to generate second sequencing data; and (d) processing the first sequencing data and the second sequencing data to determine length information of a homopolymer sequence in the template.

In some embodiments, the length information of the homopolymer sequence in the template comprises a minimum length of the homopolymer sequence. In some embodiments, the length information of the homopolymer sequence in the template comprises a total length of the homopolymer sequence.

In some embodiments, the method further comprises (e) reversing termination of the reversibly terminated bases of the second reagent mixture incorporated in the growing strand, if any, and (f) contacting the growing strand with a third reagent mixture comprising unlabeled, non-terminated bases of the first same canonical base type.

In some embodiments, the method further comprises (g) repeating (a)-(f) with a second same canonical base type different from the first canonical base type. In some embodiments, the method further comprises (h) repeating (a)-(f) with a third same canonical base type different from the first canonical base type and the second canonical base type. In some embodiments, the method further comprises (i) repeating (a)-(f) with a fourth same canonical base type different from the first canonical base type, the second canonical base type, and the third canonical base type. In some embodiments, the method further comprises (j) repeating (a)-(i) at least 10 times.

In some embodiments, the first signal is localized to a single molecule of the template. In some embodiments, the first signal is localized to a colony of molecules comprising the template.

In some embodiments, the template is immobilized to a substrate surface. In some embodiments, the template is coupled to a bead that is immobilized to the substrate surface. In some embodiments, the substrate surface comprises at least 1,000,000 individually addressable locations and the template is immobilized to an individually addressable location in the at least 1,000,000 individually addressable locations.

Another aspect of the present disclosure provides a system comprising one or more computer processors and computer memory coupled thereto. The computer memory comprises machine executable code that, upon execution by the one or more computer processors, implements any of the methods above or elsewhere herein. Another aspect of the present disclosure provides a non-transitory computer readable medium comprising machine executable code that, upon execution by one or more computer processors, implements any of the methods above or elsewhere herein.

Additional aspects and advantages of the present disclosure will become readily apparent to those skilled in this art from the following detailed description, wherein only illustrative instances of the present disclosure are shown and described. As will be realized, the present disclosure is capable of other and different instances, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein) of which:

FIG. 1 illustrates an example workflow for processing a sample for sequencing.

FIG. 2 illustrates examples of individually addressable locations distributed on substrates, as described herein.

FIG. 3 shows an example image of a substrate with a hexagonal lattice of beads, as described herein.

FIG. 4 illustrates example systems and methods for loading a sample or a reagent onto a substrate, as described herein.

FIGS. 5A-5B illustrate multiplexed stations in a sequencing system.

FIG. 6 shows components that may be used to construct labelling reagents and labeled reagents.

FIG. 7 shows examples of different types of scarred nucleotides.

FIG. 8 provides example chemical reaction schemes for capping thiol scars.

FIG. 9A provides exemplary formulae for labeled oligonucleotides.

FIG. 9B provides an exemplary schematic of a nucleic acid base coupled to a labeled oligonucleotide. Sequences shown in FIG. 9B: TVVVVVTVVVVVT (SEQ ID NO:1); ABBBBBABBBBA (SEQ ID NO:5). Additional modifications are as shown in the figure.

FIG. 10A provides an exemplary schematic of a nucleic acid base coupled to a nucleic acid structure that is further coupled to one or more labels.

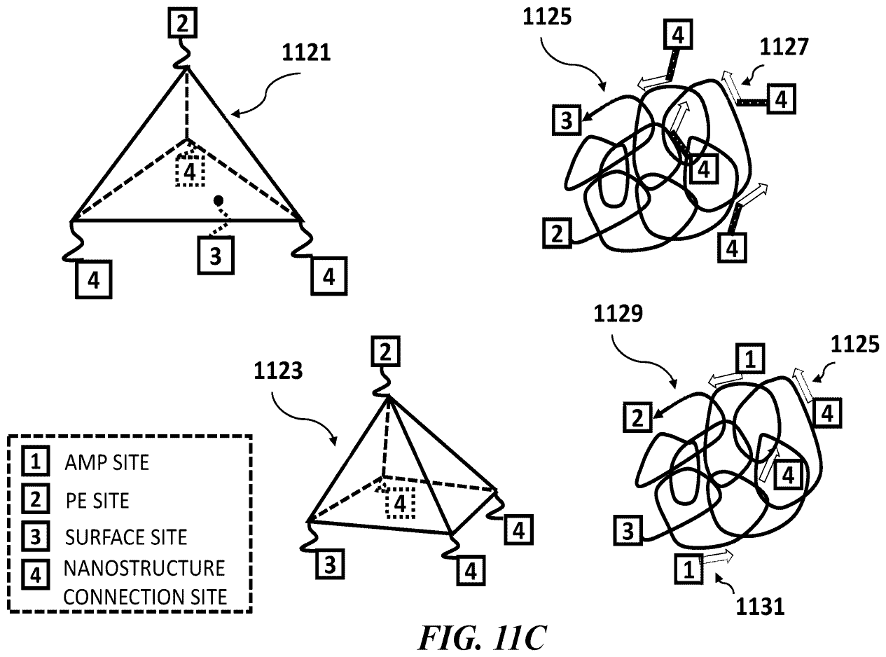

FIG. 10B provides an exemplary schematic of a nucleic acid base coupled to a label enclosed within a nucleic acid structure.

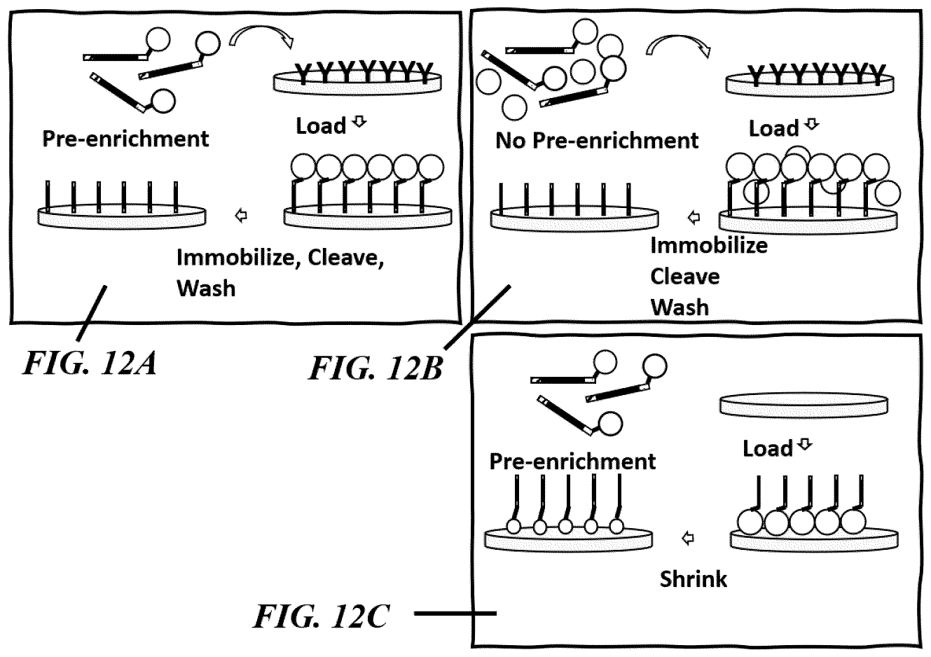

FIGS. 11A, 11B, and 11C illustrate example DNA nanostructures that can be used as supports.

FIGS. 12A, 12B, and 12C illustrate different workflows for loading nucleic acids using beads as spacers.

FIGS. 12D, 12E, and 12F illustrate different workflows for loading nucleic acids using DNA nanoballs as spacers.

FIG. 12G illustrates another example of DNA nanoball loading onto a substrate.

FIG. 13 illustrates an example flow sequencing method that can be used to generate the sequencing data described herein.

FIG. 14A illustrates a mixed-reversibly terminated sequencing method.

FIGS. 14B and 14C illustrate mixed-color sequencing methods. Sequences in FIG. 14C that are 10 nucleotides or longer: AAATTGCCCCAA (SEQ ID NO:2); TTTAACGGGG (SEQ ID NO:3).

FIG. 15 illustrates a computer system that is programmed or otherwise configured to implement methods provided herein.

FIGS. 16A and 16B show an example method for preparing a labeled nucleotide comprising a guanine analog.

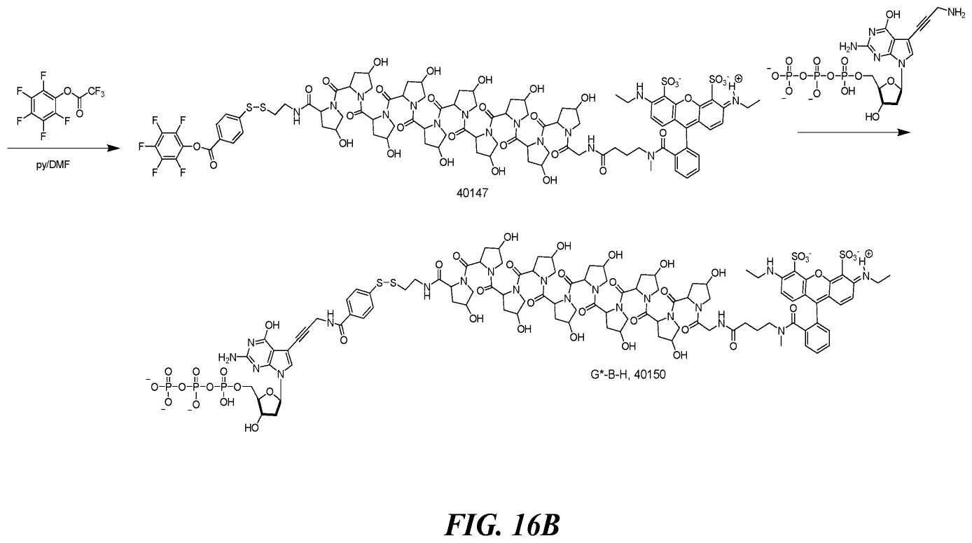

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used herein, the singular forms “a,” “an,” and “the” include the plural reference unless the context clearly dictates otherwise.

When a range of values is provided, it is to be understood that each intervening value between the upper and lower limit of that range, and any other stated or intervening value in that stated range is encompassed within the scope of the present disclosure. Where the stated range includes upper or lower limits, ranges excluding either of those included limits are also included in the present disclosure.

The term “coupled to,” as used herein, generally refers to an association between two or more objects that may be temporary or substantially permanent. A first object may be reversibly or irreversibly coupled to a second object. For example, a nucleic acid molecule may be reversibly coupled to a particle. A reversible coupling may comprise, for example, a releasable coupling (e.g., in which a first object may be released from a second object to which it is coupled). A first object releasably coupled to a second object may be separated from the second object, e.g., upon application of a stimulus, which stimulus may comprise a photostimulus (e.g., ultraviolet light), a thermal stimulus, a chemical stimulus (e.g., reducing agent), or any other useful stimulus. Coupling may encompass immobilization to a support (e.g., as described herein). Similarly, coupling may encompass attachment, such as attachment of a first object to a second object. A coupling may comprise any interaction that affects an association between two objects, including, for example, a covalent bond, a non-covalent interaction (e.g., electrostatic interaction [e.g., hydrogen bonding, ionic interaction, and halogen bonding], π-interaction [e.g., π-π interaction, polar-π interaction, cation-π interaction, and anion-π interaction], van der Waals force-based interactions [e.g., dipole-dipole interactions, dipole-induced dipole interactions, and induced dipole-induced dipole interactions], hydrophobic interaction), a magnetic interaction (e.g., magnetic dipole-dipole interaction, indirect dipole-dipole coupling), an electromagnetic interaction, adsorption, or any other useful interaction. A coupling between a first object and a second object may comprise a labile moiety, such as a moiety comprising an ester, vicinal diol, phosphodiester, peptidic, glycosidic, sulfone, Diels-Alder, or similar linkage. The strength of a coupling between a first object and a second object may be indicated by a dissociation constant (Kd) that indicates the inclination of a coupled object comprising a first object and a second object to dissociate into the uncoupled first and second objects and may be expressed as a ratio of dissociated (e.g., uncoupled) objects to coupled objects. A smaller dissociation constant is generally indicative of a stronger coupling between coupled objects. Coupled objects and their corresponding uncoupled components may exist in dynamic equilibrium with one another. For example, a solution comprising a plurality of coupled objects each comprising a first object and a second object may also include a plurality of first objects and a plurality of second objects. At a given point in time, a given first object and a given second object may be coupled to one another or the objects may be uncoupled; the relative concentrations of coupled and uncoupled components throughout the solution can depend upon the strength of the coupling between the first and second objects (reflected in the dissociation constant).

The terms “nucleotide,” “base,” or “nucleic acid base,” as used herein, generally refer to any nucleotide or nucleotide analog. The nucleotide may be naturally occurring or non-naturally occurring. The nucleotide may be a modified, synthesized, or engineered nucleotide. The nucleotide may include a canonical base or a non-canonical base. The nucleotide may comprise an alternative base. The nucleotide may include a modified polyphosphate chain (e.g., triphosphate coupled to a fluorophore). The nucleotide may comprise a label. The nucleotide may be terminated (e.g., reversibly terminated). Nonstandard nucleotides, nucleotide analogs, and/or modified analogs may include, but are not limited to, diaminopurine, 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid(v), 5-methyl-2-thiouracil, 3-(3-amino-3-N-2-carboxypropyl) uracil, (acp3)w, 2,6-diaminopurine, ethynyl nucleotide bases, 1-propynyl nucleotide bases, azido nucleotide bases, phosphoroselenoate nucleic acids and the like. In some cases, nucleotides may include modifications in their phosphate moieties, including modifications to a triphosphate moiety. Additional, non-limiting examples of modifications include phosphate chains of greater length (e.g., a phosphate chain having, 4, 5, 6, 7, 8, 9, 10 or more phosphate moieties), modifications with thiol moieties (e.g., alpha-thio triphosphate and beta-thiotriphosphates) or modifications with selenium moieties (e.g., phosphoroselenoate nucleic acids). Nucleic acids may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone. Nucleic acids may also contain amine-modified groups, such as aminoallyl-dUTP (aa-dUTP) and aminohexhylacrylamide-dCTP (aha-dCTP) to allow covalent attachment of amine reactive moieties, such as N-hydroxysuccinimide esters (NHS). Alternatives to standard DNA base pairs or RNA base pairs in the oligonucleotides of the present disclosure can provide higher density in bits per cubic mm, higher safety (resistant to accidental or purposeful synthesis of natural toxins), easier discrimination in photo-programmed polymerases, or lower secondary structure. Nucleotides may be capable of reacting or bonding with detectable moieties for nucleotide detection.

The term “terminator” as used herein with respect to a nucleotide may generally refer to a moiety that is capable of terminating primer extension. A terminator may be a reversible terminator. A reversible terminator may comprise a blocking or capping group that is attached to the 3′-oxygen atom of a sugar moiety (e.g., a pentose) of a nucleotide or nucleotide analog. Such moieties are referred to as 3′-O-blocked reversible terminators. Examples of 3′-O-blocked reversible terminators include, for example, 3′-ONH₂ reversible terminators, 3′-O-allyl reversible terminators, and 3′-O-aziomethyl reversible terminators. Alternatively, a reversible terminator may comprise a blocking group in a linker (e.g., a cleavable linker) and/or dye moiety of a nucleotide analog. 3′-unblocked reversible terminators may be attached to both the base of the nucleotide analog as well as a fluorescing group (e.g., label, as described herein). Examples of 3′-unblocked reversible terminators include, for example, the “virtual terminator” developed by Helicos BioSciences Corp. and the “lightning terminator” developed by Michael L. Metzker et al. Cleavage of a reversible terminator may be achieved by, for example, irradiating a nucleic acid molecule including the reversible terminator.

The term “sequencing,” as used herein, generally refers to a process for generating or identifying a sequence of a biological molecule, such as a nucleic acid. The sequence may be a nucleic acid sequence which comprises a sequence of nucleic acid bases. Examples of sequencing include single molecule sequencing or sequencing by synthesis. Sequencing may comprise generating sequencing signals and/or sequencing reads.

The term “misincorporation,” as used herein, generally refers to occurrences when the DNA polymerase incorporates a nucleotide, either labeled or unlabeled, that is not the correct Watson-Crick partner for the template base. Misincorporation can occur more frequently in methods that lack competition of all four bases in an incorporation event, and leads to strand loss, and thus limits the read length of a sequencing method.

The term “scar,” as used herein, generally refers to a residue left on a previously labeled nucleotide or nucleotide analog after cleavage of an optical (e.g., fluorescent) dye and, optionally, all or a portion of a linker attaching the optical dye to the nucleotide or nucleotide analog. Examples of scars include, but are not limited to, hydroxyl moieties (e.g., resulting from cleavage of an azidomethyl group, hydrocarbyldithiomethyl linkage, or 2-nitrobenzyloxy linkage), thiol moieties (e.g., resulting from cleavage of a disulfide linkage), propargyl moieties (e.g., propargyl alcohol, propargyl amine, or propargyl thiol), and benzyl moieties. For example, a scar may comprise an aromatic group such as a phenyl or benzyl group. The size and nature of a scar may affect subsequent incorporations.

Compounds and chemical moieties described herein, including linkers, may contain one or more asymmetric centers and thus give rise to enantiomers, diastereomers, and other stereoisomeric forms that are defined, in terms of absolute stereochemistry, as (R) or (S), and, in terms of relative stereochemistry, as (D)- or (L)-. The D/L system relates molecules to the chiral molecule glyceraldehyde and is commonly used to describe biological molecules including amino acids. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a phenyl ring. Separation of stereoisomers may be performed by chromatography or by forming diastereomers and separating by recrystallization, or chromatography, or any combination thereof (Jean Jacques, Andre Collet, Samuel H. Wilen, “Enantiomers, Racemates and Resolutions,” John Wiley and Sons, Inc., 1981, incorporated by reference herein in its entirety). Stereoisomers may also be obtained by stereoselective synthesis.

Compounds and chemical moieties described herein, including linkers, may exist as tautomers. A “tautomer” refers to a molecule wherein a proton shift from one atom of a molecule to another atom of the same molecule is possible. In circumstances where tautomerization is possible, a chemical equilibrium of the tautomers may exist. Unless otherwise stated, chemical structures depicted herein are intended to include structures which are different tautomers of the structures depicted. For example, the chemical structure depicted with an enol moiety also includes the keto tautomer form of the enol moiety. The exact ratio of the tautomers depends on several factors, including physical state, temperature, solvent, and pH.

Compounds and chemical moieties described herein, including linkers and dyes, may be provided in different enriched isotopic forms. For example, compounds may be enriched in the content of ²H, ³H, ¹¹C, ¹³C and/or ¹⁴C. For example, a linker, substrate (e.g., nucleotide or nucleotide analog), or dye may be deuterated in at least one position. In some examples, a linker, substrate (e.g., nucleotide or nucleotide analog), or dye may be fully deuterated. Such deuterated forms can be made by the procedure described in U.S. Pat. Nos. 5,846,514 and 6,334,997, each of which is incorporated by reference herein in its entirety. As described therein, deuteration can improve the metabolic stability and or efficacy, thus increasing the duration of action of drugs. Unless otherwise stated, structures depicted and described herein are intended to include compounds which differ only in the presence of one or more isotopically enriched atoms. For example, compounds and chemical moieties having the present structures except for the replacement of a hydrogen by a deuterium or tritium, or the replacement of a carbon by ¹³C- or ¹⁴C-enriched carbon are within the scope of the present disclosure. The compounds and chemical moieties of the present disclosure may contain unnatural proportions of atomic isotopes at one or more atoms that constitute such compounds. For example, a compound or chemical moiety such as a linker, substrate (e.g., nucleotide or nucleotide analog), or dye, or a combination thereof, may be labeled with one or more isotopes, such as deuterium (²H), tritium (H), iodine-125 (¹²⁵I) or carbon14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, and ¹²⁵I are all contemplated. All isotopic variations of the compounds and chemical moieties described herein whether radioactive or not are encompassed within the scope of the present disclosure.

The term “analyte,” as used herein, generally refers to an object that is the subject of analysis, or an object, regardless of being the subject of analysis, that is directly or indirectly analyzed during a process. An analyte may be synthetic. An analyte may be, originate from, and/or be derived from, a sample, such as a biological sample. In some examples, an analyte is or includes a molecule, macromolecule (e.g., nucleic acid, carbohydrate, protein, lipid, etc.), nucleic acid, carbohydrate, lipid, antibody, antibody fragment, antigen, peptide, polypeptide, protein, macromolecular group (e.g., glycoproteins, proteoglycans, ribozymes, liposomes, etc.), cell, tissue, biological particle, or an organism, or any engineered copy or variant thereof, or any combination thereof. The term “processing an analyte,” as used herein, generally refers to one or more stages of interaction with one more samples. Processing an analyte may comprise conducting a chemical reaction, biochemical reaction, enzymatic reaction, hybridization reaction, polymerization reaction, physical reaction, any other reaction, or a combination thereof with, in the presence of, or on, the analyte. Processing an analyte may comprise physical and/or chemical manipulation of the analyte. For example, processing an analyte may comprise detection of a chemical change or physical change, addition of or subtraction of material, atoms, or molecules, molecular confirmation, detection of the presence of a fluorescent label, detection of a Forster resonance energy transfer (FRET) interaction, or inference of absence of fluorescence.

The term “biological sample,” as used herein, generally refers to any sample derived from a subject or specimen. The biological sample can be a fluid, tissue, collection of cells (e.g., cheek swab), hair sample, or feces sample. The fluid can be blood (e.g., whole blood), saliva, urine, or sweat. The tissue can be from an organ (e.g., liver, lung, or thyroid), or a mass of cellular material, such as, for example, a tumor. The biological sample can be a cellular sample or cell-free sample. Examples of biological samples include nucleic acid molecules, amino acids, polypeptides, proteins, carbohydrates, fats, or viruses. In an example, a biological sample is a nucleic acid sample including one or more nucleic acid molecules, such as deoxyribonucleic acid (DNA) and/or ribonucleic acid (RNA). The nucleic acid sample may comprise cell-free nucleic acid molecules, such as cell-free DNA or cell-free RNA. Non-limiting examples of nucleic acids include DNA, RNA, genomic DNA or synthetic DNA/RNA or coding or non-coding regions of a gene or gene fragment, loci (locus) defined from linkage analysis, exons, introns, messenger RNA (mRNA), transfer RNA, ribosomal RNA (rRNA), short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, and isolated RNA of any sequence. Further, samples may be extracted from variety of animal fluids containing cell free sequences, including but not limited to blood, serum, plasma, vitreous, sputum, urine, tears, perspiration, saliva, semen, mucosal excretions, mucus, spinal fluid, amniotic fluid, lymph fluid and the like. Cell free polynucleotides may be fetal in origin (via fluid taken from a pregnant subject) or may be derived from tissue of the subject itself. A biological sample may also refer to a sample engineered to mimic one or more properties (e.g., nucleic acid sequence properties, e.g., sequence identity, length, GC content, etc.) of a sample derived from a subject or specimen.

As used herein, the term “template nucleic acid” generally refers to the nucleic acid to be sequenced. The template nucleic acid may be a polynucleotide. The template nucleic acid may be an analyte or be associated with an analyte. For example, the analyte can be a mRNA, and the template nucleic acid is the mRNA, or a cDNA derived from the mRNA, or other derivative thereof. In another example, the analyte can be a protein, and the template nucleic acid is an oligonucleotide that is conjugated to an antibody that binds to the protein, or derivatives thereof. Sequencing may be performed on template nucleic acids immobilized on a support, such as a flow cell, substrate, and/or one or more beads. In some cases, a template nucleic acid may be amplified to produce a colony of nucleic acid molecules attached to the support to produce amplified sequencing signals. In one example, (i) a template nucleic acid is subjected to a nucleic acid reaction, e.g., amplification, to produce a clonal population of the nucleic acid attached to a bead, the bead immobilized to a substrate, (ii) amplified sequencing signals from the immobilized bead are detected from the substrate surface during or following one or more nucleotide flows, and (iii) the sequencing signals are processed to generate sequencing reads. The substrate surface may immobilize multiple beads at distinct locations, each bead containing distinct colonies of nucleic acids, and upon detecting the substrate surface, multiple sequencing signals may be simultaneously or substantially simultaneously processed from the different immobilized beads at the distinct locations to generate multiple sequencing reads. In some sequencing methods, the nucleotide flows comprise non-terminated nucleotides. In some sequencing methods, the nucleotide flows comprise terminated nucleotides.

The term “nucleotide flow” as used herein, generally refers to a temporally distinct instance of providing a nucleotide-containing reagent to a sequencing reaction space. The term “flow” as used herein, when not qualified by another reagent, generally refers to a nucleotide flow. For example, providing two flows may refer to (i) providing a nucleotide-containing reagent (e.g., an A-base-containing solution) to a sequencing reaction space at a first time point and (ii) providing a nucleotide-containing reagent (e.g., G-base-containing solution) to the sequencing reaction space at a second time point different from the first time point. A “sequencing reaction space” may be any reaction environment comprising a template nucleic acid. For example, the sequencing reaction space may be or comprise a substrate surface comprising a template nucleic acid immobilized thereto; a substrate surface comprising a bead immobilized thereto, the bead comprising a template nucleic acid immobilized thereto; or any reaction chamber or surface that comprises a template nucleic acid, which may or may not be immobilized. A nucleotide flow can have any number of base types (e.g., A, T, G, C; or U), for example 1, 2, 3, or 4 canonical base types. A “flow order,” as used herein, generally refers to the order of nucleotide flows used to sequence a template nucleic acid. A flow order may be expressed as a one-dimensional matrix or linear array of bases corresponding to the identities of, and arranged in chronological order of, the nucleotide flows provided to the sequencing reaction space:
(e.g., [A, T, G, C, A, T, G, C, A, T, G, A, T, G, A, T, G, A, T, G, C, A, T, G, C]).

Such one-dimensional matrix or linear array of bases in the flow order may also be referred to herein as a “flow space.” A flow order may have any number of nucleotide flows. A “flow position,” as used herein, generally refers to the sequential position of a given nucleotide flow entry in the flow space (e.g., an element in the one-dimensional matrix or linear array). A “flow cycle,” as used herein, generally refers to the order of nucleotide flow(s) of a sub-group of contiguous nucleotide flow(s) within the flow order. A flow cycle may be expressed as a one-dimensional matrix or linear array of an order of bases corresponding to the identities of, and arranged in chronological order of, the nucleotide flows provided within the sub-group of contiguous flow(s) (e.g., [A, T, G, C], [A, A, T, T, G, G, C, C], [A, T], [A/T, A/G], [A, A], [A], [A, T, G], etc.). A flow cycle may have any number of nucleotide flows. A given flow cycle may be repeated one or more times in the flow order, consecutively or non-consecutively. Accordingly, the term “flow cycle order,” as used herein, generally refers to an ordering of flow cycles within the flow order and can be expressed in units of flow cycles. For example, where [A, T, G, C] is identified as a 1st flow cycle, and [A T G] is identified as a 2^{nd} flow cycle, the flow order of [A, T, G, C, A, T, G, C, A, T, G, A, T, G, A, T, G, A, T, G, C, A, T, G, C] may be described as having a flow-cycle order of [1^{st} flow cycle; 1^{st} flow cycle; 2^{nd} flow cycle; 2^{nd} flow cycle; 2^{nd} flow cycle; 1^{st} flow cycle; 1^{st} flow cycle]. Alternatively or in addition, the flow cycle order may be described as [cycle 1, cycle 2, cycle 3, cycle 4, cycle 5, cycle 6], where cycle 1 is the 1^{st} flow cycle, cycle 2 is the 1^{st} flow cycle, cycle 3 is the 2^{nd} flow cycle, etc. In some cases, a flow-cycle order may be [T G C A]. However, any other permutation of nucleotides T (or U), G, C, and A may be used as a flow-cycle order.

### Sample Processing Methods

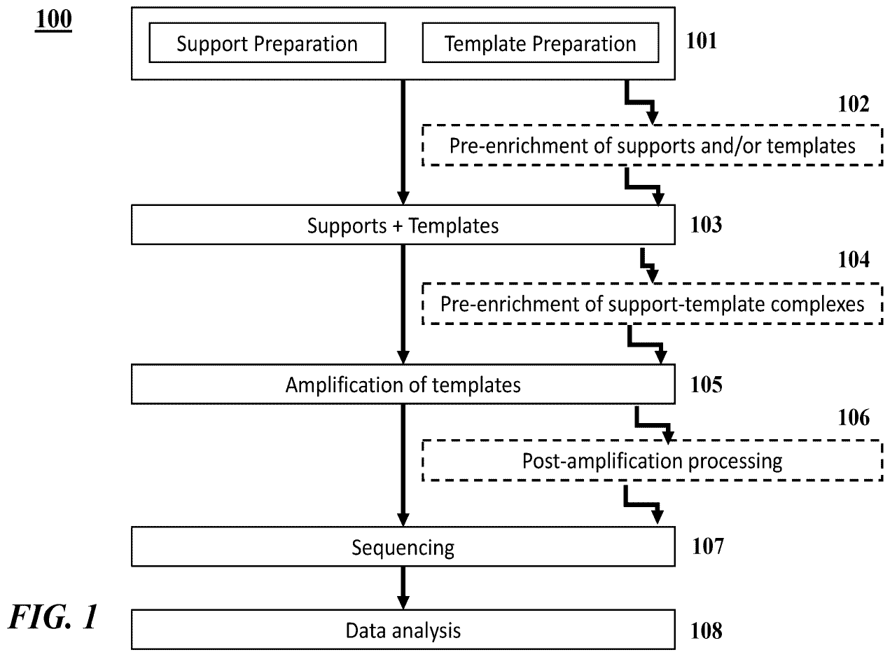

Described herein are devices, systems, methods, compositions, and kits for processing samples, such as to prepare a sample for sequencing, to sequence a sample, and/or to analyze sequencing data. FIG. 1 illustrates an example sequencing workflow 100, according to the devices, systems, methods, compositions, and kits of the present disclosure.

Supports and/or template nucleic acids may be provided and/or prepared (101) to be compatible with downstream sequencing operations (e.g., 107). A support (e.g., bead) may help immobilize a template nucleic acid to a substrate, such as when the template nucleic acid is coupled to the support, and the support is in turn immobilized to the substrate. The support may further function as a binding entity to retain derivatives (e.g., amplification products) from a single template nucleic acid together for downstream processing, such as for sequencing operations. This may be useful in distinguishing a colony from other colonies (e.g., on other supports) and generating amplified sequencing signals corresponding to a template nucleic acid. A support may comprise an oligonucleotide comprising one or more functional nucleic acid sequences. The oligonucleotide may be single-stranded, double-stranded, or partially double-stranded. For example, the oligonucleotide may comprise a capture sequence, a primer sequence, a sequencing primer sequence, a barcode sequence, a sample index sequence, a unique molecular identifier (UMI), a flow cell adapter sequence, an adapter sequence, a target sequence, a random sequence, a binding sequence (e.g., for a splint, primer, template nucleic acid, capture sequence, etc.), or any other functional sequence useful for a downstream operation, a complement thereof, or any combination thereof. The capture sequence may be configured to hybridize to a sequence of a template nucleic acid or derivative thereof. The support may comprise a plurality of oligonucleotides, for example on the order of 10, 10², 10³, 10⁴, 10⁵, 10⁶, 10⁷, or more molecules. The support may comprise a single species of oligonucleotide which comprise identical sequences. The support may comprise multiple species of oligonucleotides which have varying sequences. In some cases, the support comprises a single species of a primer (e.g., forward primer) for amplification. In some cases, the support comprises two species of primer (e.g., forward primer, reverse primer) for amplification. Devices, systems, methods, compositions, and kits for preparing and using support species are described in further detail in U.S. Patent Pub. No. 20220042072A1 and International Patent Pub. No. WO2022040557A2, each of which is entirely incorporated herein by reference for all purposes.

A support may comprise one or more capture entities, where a capture entity is configured for capture by a capturing entity. A capture entity may be coupled to or be part of an oligonucleotide coupled to the support. A capture entity may be coupled to or be part of the support. Examples of capture entity-capturing entity pairs and capturing entity-capture entity pairs include streptavidin (SA)-biotin; complementary sequences; magnetic particle-magnetic field system; charged particle-electric field system; azide-cyclooctyne; thiol-maleimide; click chemistry pairs; cross-linking pairs; etc. The capture entity-capturing entity pair may comprise one or more chemically modified bases. A capture entity and capturing entity may bind, couple, hybridize, or otherwise associate with each other. The association may comprise formation of a covalent bond, non-covalent bond, releasable bond (e.g., cleavable bond that is cleavable upon application of a stimulus), and/or no bond. The capture entity may be capable of linking to a nucleotide. In some instances, the capturing entity may comprise a secondary capture entity, e.g., for subsequent capture by a secondary capturing entity. The secondary capture entity-secondary capturing entity pair may comprise any one or more of the capturing mechanisms described elsewhere herein.

A support may comprise one or more cleavable moieties, also referred to herein as excisable moieties. The cleavable moiety may be coupled to or be part of an oligonucleotide coupled to the support. The cleavable moiety may be coupled to the support. A cleavable moiety may comprise any useful moiety that can be used to cleave an oligonucleotide (or portion thereof) from the support, or otherwise release a nucleic acid strand from the support and/or the oligonucleotide. A cleavable moiety may comprise a uracil, ribonucleotide, methylated nucleotide, or another modified nucleotide that is excisable or cleavable using an enzyme (e.g., UDG, RNAse, APE1, MspJI, endonuclease, exonuclease, etc.). The cleavable moiety may comprise an abasic site or an analog of an abasic site (e.g., dSpacer), a dideoxyribose, a spacer, e.g., C3 spacer, hexanediol, triethylene glycol spacer (e.g., Spacer 9), hexa-ethyleneglycol spacer (e.g., Spacer 18), a photocleavable moiety, or combinations or analogs thereof. Alternatively, or in addition, the cleavable moiety may be cleavable using one or more stimuli, e.g., photostimulus, chemical stimulus, thermal stimulus, etc.

The sequencing workflow 100 may not involve supports, for example when a template nucleic acid and/or its derivatives are directly attached to a substrate and amplified and/or sequenced from the substrate.

A template nucleic acid may include an insert sequence sourced from a biological sample. The template nucleic acid may be derived from any nucleic acid of the biological sample (e.g., endogenous nucleic acid) and result from any number of processing operations, such as but not limited to fragmentation, degradation or digestion, transposition, ligation, reverse transcription, extension, replication, etc. The template nucleic acid may be single-stranded, double-stranded, or partially double-stranded. A template nucleic acid may comprise one or more functional nucleic acid sequences. For example, the template nucleic acid may comprise a capture sequence, a primer sequence, a sequencing primer sequence, a barcode sequence, a sample index sequence, a unique molecular identifier (UMI), a flow cell adapter sequence, an adapter sequence, a target sequence, a random sequence, a binding sequence (e.g., for a splint, primer, template nucleic acid, capture sequence, etc.), or any other functional sequence useful for a downstream operation, a complement thereof, or any combination thereof. The template nucleic acid may comprise an adapter sequence configured to be captured by a capture sequence of an oligonucleotide coupled to a support. The one or more functional nucleic acid sequences may be disposed at one end or both ends of the insert sequence. A nucleic acid molecule comprising the insert sequence or complement thereof may be processed with (e.g., attached to, extended from, etc.) one or more adapter molecules to generate the template nucleic acid comprising the insert sequence and one or more functional nucleic acid sequences. A template nucleic acid may comprise one or more capture entities and/or one or more cleavable moieties that are described elsewhere herein.

Optionally, the supports and/or template nucleic acids may be pre-enriched (102). For example, a support comprising a distinct oligonucleotide sequence is pre-enriched to isolate from a mixture comprising support(s) that do not have the distinct oligonucleotide sequence. For example, a template nucleic acid comprising a distinct configuration (e.g., comprising a particular adapter sequence) is pre-enriched to isolate from a mixture comprising template nucleic acids that do not have the distinct configuration. In some cases, the capture entity on the supports and/or template nucleic acids are used for pre-enrichment.

The supports and template nucleic acids may be attached (103) to generate support-template complexes. A template nucleic acid may be coupled to a support via any method(s) that results in a stable association between the template nucleic acid and the support. For example, the template nucleic acid may hybridize to an oligonucleotide on the support; the template nucleic acid may be ligated to a nucleic acid coupled to the support; the template nucleic acid may hybridize to one or more intermediary molecules, such as a splint, bridge, and/or primer molecule, which hybridizes to an oligonucleotide on the support; and/or the template nucleic acid may be hybridized to an oligonucleotide on a support, which oligonucleotide comprises a primer sequence which is extended. In some cases, the respective concentrations of the supports and template nucleic acids may be adjusted such that a majority of support-template complexes are single template-attached supports (e.g., a support attached to a single template nucleic acid).

Optionally, support-template complexes may be pre-enriched (104), wherein a support-template complex is isolated from a mixture comprising support(s) and/or template nucleic acid(s) that are not attached to each other. In some cases, the capture entity on the supports and/or template nucleic acids are used for pre-enrichment.

The template nucleic acids may be subjected to amplification reactions (105) to generate a plurality of amplification products immobilized to the support. Such amplification reactions may comprise performing polymerase chain reaction (PCR) or any other amplification methods described herein, including but not limited to emulsion PCR (ePCR or emPCR), isothermal amplification, recombinase polymerase amplification (RPA), rolling circle amplification (RCA), multiple displacement amplification (MDA), bridge amplification, template walking, etc. Amplification reactions can occur while the support is immobilized to a substrate. Amplification reactions can occur off the substrate, such as in solution, or on a different surface or platform. Amplification reactions can occur in isolated reaction volumes, such as within multiple droplets in an emulsion during emulsion PCR (ePCR or emPCR), or in wells or tubes.

Optionally, subsequent to amplification, the supports, template nucleic acids, and/or support-template complexes may be subjected to post-amplification processing (106). Often, subsequent to amplification, a resulting mixture may comprise a mix of positive supports (e.g., those comprising a template nucleic acid molecule) and negative supports (e.g., those not attached to template nucleic acid molecules). Enrichment procedure(s) may isolate positive supports from the mixtures. Example methods of enrichment of amplified supports are described in U.S. Pat. Nos. 10,900,078 and 11,118,223, and U.S. patent application Ser. No. 18/176,418, each of which is incorporated by reference herein in its entirety.

The template nucleic acids may be subject to sequencing (107). The template nucleic acid(s) may be sequenced while attached to the support. Alternatively, the template nucleic acid molecules may be free of the support when sequenced and/or analyzed. The template nucleic acids may be sequenced while immobilized to a substrate, such as via a support or otherwise. Examples of substrate-based sample processing systems are described elsewhere herein. Any sequencing method may be used, for example pyrosequencing, single molecule sequencing, sequencing by synthesis (SBS), sequencing by ligation, sequencing by binding, etc.

For example, sequencing comprises extending a sequencing primer (or growing strand) hybridized to a template nucleic acid by providing labeled nucleotide reagents, washing away unincorporated nucleotides from the reaction space, and detecting one or more signals from the labeled nucleotide reagents which are indicative of an incorporation event or lack thereof. After detection, the labels may be cleaved and the whole process may be repeated any number of times to determine sequence information of the template nucleic acid. One or more intermediary flows may be provided intra- or inter-repeat, such as washing flows, label cleaving flows, terminator cleaving flows, reaction-completing flows (e.g., double tap flow, triple tap flow, etc.), labeled flows (or bright flows), unlabeled flows (or dark flows), phasing flows, chemical scar capping flows, etc. A nucleotide mixture that is provided during any one flow may comprise only labeled nucleotides, only unlabeled nucleotides, or a mixture of labeled and unlabeled nucleotides. The mixture of labeled and unlabeled nucleotides may be of any fraction of labeled nucleotides, such as at least or at most 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 99%. A nucleotide mixture that is provided during any one flow may comprise only non-terminated nucleotides, only terminated nucleotides, or a mixture of terminated and non-terminated nucleotides. When using only non-terminated nucleotides, terminator cleaving flows may be omitted from the sequencing process. When using terminated nucleotides, to proceed with the next step of extension, prior to, during, or subsequent to detection, a terminator cleaving flow may be provided to cleave blocking moieties. A nucleotide mixture that is provided during any one flow may comprise any number of canonical base types (e.g., A, T, G, C, U), such as a single canonical base type, two canonical base types, three canonical base types, four canonical base types or five canonical base types (including T and U). Different types of nucleotide bases may be flowed in any order and/or in any mixture of base types that is useful for sequencing. Various flow-based sequencing systems and methods are described in U.S. Pat. No. 11,459,609, which is incorporated by reference herein in its entirety. In some cases, nucleotides of different canonical base types may be labeled and detectable at a single frequency (e.g., using the same or different dyes). In other cases, nucleotides of different canonical base types may be labeled and detectable at different frequencies (e.g., using the same or different dyes).

Subsequent to sequencing, the sequencing signals collected and/or generated may be subjected to data analysis (108). The sequencing signals may be processed to generate base calls and/or sequencing reads. In some cases, the sequencing reads may be processed to generate diagnostics data to the biological sample, or the subject from which the biological sample was derived from. The data analysis may comprise image processing, alignment to a genome or reference genome, training and/or trained algorithms, error correction, and the like.

While the sequencing workflow 100 with respect to FIG. 1 has been described with respect to the use of supports to bind template molecules, it will be appreciated that the different supports may be effectively replaced by using spatially distinct locations on one or more surfaces, which do not necessarily have to be the surfaces of individual supports (e.g., beads). For example, a first spatially distinct location on a surface may be capable of directly immobilizing a first colony of a first template nucleic acid and a second spatially distinct location on the same surface (or a different surface) may be capable of directly immobilizing a second colony of a second template nucleic acid to distinguish from the first colony. In some cases, the surface comprising the spatially distinct locations may be a surface of the substrate on which the sample is sequenced, thus streamlining the amplification-sequencing workflow.

It will be appreciated that in some instances, the different operations described in the sequencing workflow 100 may be performed in a different order. It will be appreciated that in some instances, one or more operations described in the sequencing workflow 100 may be omitted or replaced with other comparable operation(s). It will be appreciated that in some instances, one or more additional operations described in the sequencing workflow 100 may be performed. The different operations described with respect to sequencing workflow 100 may be performed with the help of open substrate systems described herein.

### Open Substrate Systems

Described herein are devices, systems, and methods that use open substrates or open flow cell geometries to process a sample. The term “open substrate,” as used herein, generally refers to a substrate in which any point on an active surface of the substrate is physically accessible from a direction normal to the substrate. The devices, systems and methods may be used to facilitate any application or process involving a reaction or interaction between two objects, such as between an analyte and a reagent or between two reagents. For example, the reaction or interaction may be chemical (e.g., polymerase reaction) or physical (e.g., displacement). The devices, systems, and methods described herein may benefit from higher efficiency (e.g., faster reagent delivery and lower volumes of reagents required per surface area), shorter completion time, use of fewer resources (e.g., various reagents), and/or reduced system costs. The devices, systems, and methods described herein may avoid contamination problems common to microfluidic channel flow cells that are fed from multiport valves which can be a source of carryover from one reagent to the next. The open substrates or flow cell geometries may be used to process any analyte from any sample, such as but not limited to, nucleic acid molecules, protein molecules, antibodies, antigens, cells, and/or organisms, as described herein. The open substrates or flow cell geometries may be used for any application or process, such as, but not limited to, sequencing by synthesis, sequencing by ligation, amplification, proteomics, single cell processing, barcoding, and sample preparation, as described herein.

A sample processing system may comprise a substrate, and devices and systems that perform one or more operations with or on the substrate. The sample processing system may permit highly efficient dispensing of analytes and reagents onto the substrate. The sample processing may permit highly efficient imaging of one or more analytes, or signals corresponding thereto, on the substrate. The sample processing system may comprise an imaging system comprising a detector. Substrates, detectors, and sample processing hardware that can be used in the sample processing system are described in further detail in U.S. Patent Pub. No. 20200326327A1, U.S. Patent Pub. No. 20210079464A1, International Patent Pub. No. WO2022072652A1, and U.S. Patent Pub. No. 20210354126A1, each of which is entirely incorporated herein by reference for all purposes.

### Substrates

The substrate may be a solid substrate. The substrate may entirely or partially comprise one or more of rubber, glass, silicon, a metal such as aluminum, copper, titanium, chromium, or steel, a ceramic such as titanium oxide or silicon nitride, a plastic such as polyethylene (PE), low-density polyethylene (LDPE), high-density polyethylene (HDPE), polypropylene (PP), polystyrene (PS), high impact polystyrene (HIPS), polyvinyl chloride (PVC), polyvinylidene chloride (PVDC), acrylonitrile butadiene styrene (ABS), polyacetylene, polyamides, polycarbonates, polyesters, polyurethanes, polyepoxide, polymethyl methacrylate (PMMA), polytetrafluoroethylene (PTFE), phenol formaldehyde (PF), melamine formaldehyde (MF), urea-formaldehyde (UF), polyetheretherketone (PEEK), polyetherimide (PEI), polyimides, polylactic acid (PLA), furans, silicones, polysulfones, any mixture of any of the preceding materials, or any other appropriate material. The substrate may be entirely or partially coated with one or more layers of a metal (e.g., aluminum, copper, silver, or gold), an oxide (e.g., silicon oxide, SiₓO_{y}, where x, y may take on any possible values), a photoresist (e.g., SU8), an aminosilane or hydrogel, polyacrylic acid, polyacrylamide dextran, polyethylene glycol (PEG), or any combination of any of the preceding materials, or any other appropriate coating. The substrate may comprise multiple layers of the same or different types of material. The substrate may be fully or partially opaque or transparent to visible light. A surface of the substrate may be modified to comprise active chemical groups, such as amines, esters, hydroxyls, epoxides, and the like, or a combination thereof. A surface of the substrate may be modified to comprise any of the binders or linkers described herein. In some instances, such binders, linkers, active chemical groups, and the like may be added as an additional layer or coating to the substrate.

The substrate may have the general form of a cylinder, cylindrical shell, disk, rectangular prism, or any other geometric form. The substrate may have a thickness (e.g., a minimum dimension) of at least and/or at most about 100 micrometers (μm), 200 μm, 500 μm, 1 millimeter (mm), 2 mm, 5 mm, 10 mm, 15 mm, 20 mm, 25 mm, 30 mm, 35 mm, 40 mm, 45 mm, 50 or mm. The substrate may have a first lateral dimension (such as a width for a substrate having the general form of a rectangular prism or a radius or diameter for a substrate having the general form of a cylinder) and/or a second lateral dimension (such as a length for a substrate having the general form of a rectangular prism) of at least and/or at most about 1 mm, 2 mm, 5 mm, 10 mm, 20 mm, 30 mm, 40 mm, 50 mm, 100 mm, 150 mm, 200 mm, 300 mm, 400 mm, 500 mm, 1,000 mm, 1,500 mm, 2,000 mm, 2,500 mm, 3,000 mm, 4,000 mm, 5,000 mm or more.

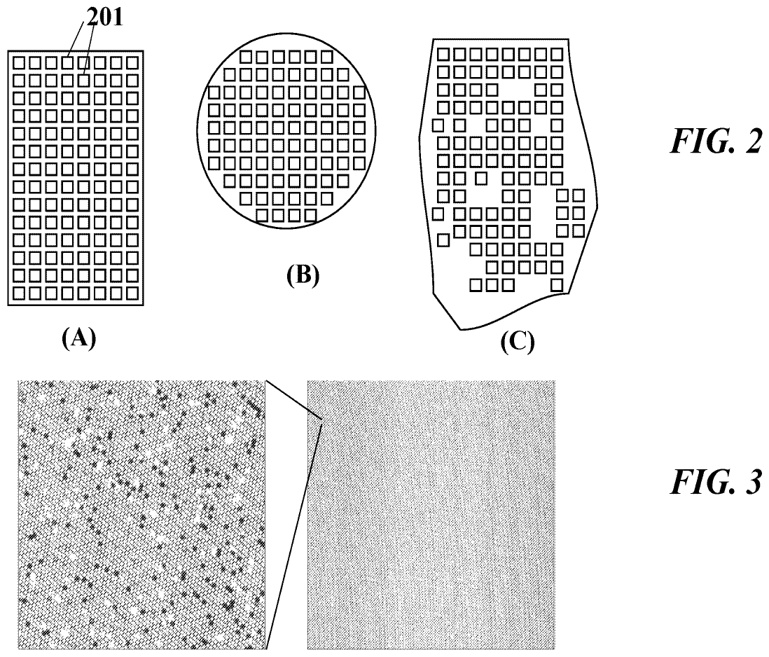

The substrate may comprise a plurality of individually addressable locations. The individually addressable locations may comprise locations that are physically accessible for manipulation (e.g., placement, extraction, reagent dispensing, seeding, heating, cooling, or agitation). The manipulation may be accomplished through, e.g., localized microfluidic, pipet, optical, laser, acoustic, magnetic, and/or electromagnetic interactions with the analyte or its surroundings. The individually addressable locations may comprise locations that are digitally accessible (e.g., each individually addressable location may be located, identified, and/or accessed electronically or digitally for indexing, mapping, sensing, associating with a device (e.g., detector, processor, dispenser, etc.), or other processing). In some cases, the individually addressable locations may be defined by physical features of the substrate (e.g., on a modified surface) to distinguish individually addressable locations from each other and from non-individually addressable locations. In some cases, the individually addressable locations may not be defined by physical features of the substrate, and instead may be defined digitally (e.g., by indexing) and/or via the analytes and/or reagents that are loaded on the substrate (e.g., the locations in which analytes are immobilized on the substrate). The plurality of individually addressable locations may be arranged as an array, randomly, or according to any pattern on the substrate. FIG. 2 illustrates top views of different substrates comprising different arrangements of individually addressable locations 201. Panel A shows a substantially rectangular substrate with regular linear array, panel B shows a substantially circular substrate with regular linear array, and panel C shows an irregularly shaped substrate with irregular placement of locations.

The substrate may have any number of individually addressable locations, for example, on the order of 1, 10¹, 10², 10³, 10⁴, 10⁵, 10⁶, 10⁷, 10⁸, 10⁹, 10¹⁰, 10¹¹, 10¹², 10¹³ or more individually addressable locations. Each individually addressable location may have any shape or form, for example the general shape or form of a circle, oval, square, rectangle, polygonal, or non-polygonal shape when viewed from the top. A plurality of individually addressable locations can have uniform shape or form, or different shapes or forms. An individually addressable location may have any size. In some cases, an individually addressable location may have an area of at least and/or at most about 0.1, 0.2, 0.25, 0.3, 0.4, 0.5, 0.6, 0.7, 0.8, 0.9, 1, 1.1, 1.2, 1.25, 1.3, 1.4, 1.5, 1.6, 1.7, 1.75, 1.8, 1.9, 2, 2.25, 2.5, 2.75, 3, 3.25, 3.5, 3.75, 4, 4.25, 4.5, 4.75, 5, 5.5, 6, 7, 8, 9, 10 square microns (μm²), or more. The individually addressable locations may be distributed on a substrate with a pitch determined by the distance between the center of a first location and the center of the closest or neighboring individually addressable location. Locations may be spaced with a pitch of at least and/or at most about 0.1, 0.2, 0.25, 0.3, 0.4, 0.5, 0.6, 0.7, 0.8, 0.9, 1, 1.1, 1.2, 1.25, 1.3, 1.4, 1.5, 1.6, 1.7, 1.75, 1.8, 1.9, 2, 2.25, 2.5, 2.75, 3, 3.25, 3.5, 3.75, 4, 4.25, 4.5, 4.75, 5, 5.5, 6, 6.5, 7, 7.5, 8, 8.5, 9, 9.5, or 10 micron (μm). In some cases, the pitch between two individually addressable locations may be determined as a function of a size of a loading object (e.g., bead). For example, where the loading object is a bead having a maximum diameter, the pitch may be at least about the maximum diameter of the loading object.

Each of the plurality of individually addressable locations, or each of a subset of the locations, may be capable of immobilizing thereto an analyte (e.g., a nucleic acid, a protein, a carbohydrate, etc.) or a reagent (e.g., a nucleic acid, a probe molecule, a barcode molecule, an antibody molecule, a primer molecule, a bead, etc.). An analyte or reagent may be immobilized to an individually addressable location via a support, such as a bead. For example, a first bead comprising a first colony of nucleic acid molecules each comprising a first sequence is immobilized to a first individually addressable location, and a second bead comprising a second colony of nucleic acid molecules each comprising a second sequence is immobilized to a second individually addressable location. A substrate may comprise more than one type of individually addressable location arranged as an array, randomly, or according to any pattern on the substrate. Different types of individually addressable locations may have different chemical, physical, and/or biological properties (e.g., hydrophobicity, charge, color, topography, size, dimensions, geometry, etc.).

An individually addressable location may comprise a distinct surface chemistry. The distinct surface chemistry may distinguish between different addressable locations and/or distinguish an individually addressable location from surrounding locations. For example, a first location type may comprise a first surface chemistry, and a second location type may lack the first surface chemistry and/or may comprise a second, different surface chemistry. A first location type may have a first affinity towards an object (e.g., a bead comprising nucleic acid molecules, e.g., amplicons, immobilized thereto) and a second location type may have a second, different affinity towards the same object. In other examples, a first location type comprising a first surface chemistry may have an affinity towards a first sample type (e.g., a bead comprising nucleic acid molecules, e.g., amplicons, immobilized thereto) and exclude a second sample type (e.g., a bead lacking nucleic acid molecules, e.g., amplicons, immobilized thereto). The first location type and the second location type may or may not be disposed on the surface in alternating fashion. A first location type or region type may comprise a positively charged surface chemistry and a second location type or region type may comprise a negatively charged surface chemistry. A first location type or region type may comprise a hydrophobic surface chemistry and a second location type or region type may comprise a hydrophilic surface chemistry. A first location type may comprise a binder, as described elsewhere herein, and a second location type may not comprise the binder or may comprise a different binder. In some cases, a surface chemistry may comprise an amine. In some cases, a surface chemistry may comprise a silane (e.g., tetramethylsilane). In some cases, the surface chemistry may comprise hexamethyldisilazane (HMDS). In some cases, the surface chemistry may comprise (3-aminopropyl)triethoxysilane (APTMS). In some cases, the surface chemistry may comprise a surface primer molecule or any oligonucleotide molecule that has any degree of affinity towards another molecule. In one example, the substrate comprises a plurality of individually addressable locations, each defined by APTMS, which are positively charged and has affinity towards an amplified bead (e.g., a bead comprising nucleic acid molecules, e.g., amplicons, immobilized thereto) which exhibits a negative charge. The locations surrounding the plurality of individually addressable locations may comprise HMDS which repels amplified beads.

In some cases, the individually addressable locations may be indexed, e.g., spatially. Data corresponding to an indexed location, collected over multiple periods of time, may be linked to the same indexed location. In some cases, sequencing signal data collected from an indexed location, during iterations of sequencing-by-synthesis flows, are linked to the indexed location to generate a sequencing read for an analyte immobilized at the indexed location. In some embodiments, the individually addressable locations are indexed by demarcating part of the surface, such as by etching or notching the surface, using a dye or ink, depositing a topographical mark, depositing a sample (e.g., a control nucleic acid sample), depositing a reference object (e.g., e.g., a reference bead that always emits a detectable signal during detection), and the like, and the individually addressable locations may be indexed with reference to such demarcations. A combination of positive demarcations and negative demarcations (lack thereof) may be used to index the individually addressable locations. In some embodiments, each of the individually addressable locations is indexed. In some embodiments, a subset of the individually addressable locations is indexed. In some embodiments, the individually addressable locations are not indexed, and a different region of the substrate is indexed.

The substrate may comprise a planar or substantially planar surface. Substantially planar may refer to planarity at a micrometer level (e.g., a range of unevenness on the planar surface does not exceed the micrometer scale) or nanometer level (e.g., a range of unevenness on the planar surface does not exceed the nanometer scale). Alternatively, substantially planar may refer to planarity at less than a nanometer level or greater than a micrometer level (e.g., millimeter level). Alternatively or in addition, a surface of the substrate may be textured or patterned. For example, the substrate may comprise grooves, troughs, hills, pillars, wells, cavities (e.g., micro-scale cavities or nano-scale cavities), and/or channels. The substrate may have regular textures and/or patterns across the surface of the substrate. The substrate may have regular geometric structures (e.g., wedges, cuboids, cylinders, spheroids, hemispheres, etc.) above or below a reference level of the surface. Alternatively, the substrate may have irregular textures and/or patterns across the surface of the substrate. The substrate may be textured or patterned such that all features are at or above a reference level of the surface (no features below a reference level of the surface, such as a well). The substrate may be textured or patterned such that all features are at or below a reference level of the surface (no features below a reference level of the surface, such as a pillar). In some instances, a texture of the substrate may comprise structures having a maximum dimension of at most about 500%, 400%, 300%, 200%, 100%, 90%, 80%, 70%, 60%, 50%, 40%, 30%, 20%, 10%, 9%, 8%, 7%, 6%, 5%, 4%, 3%, 2%, 1%, 0.1%, 0.01%, 0.001%, 0.0001%, 0.00001% of the total thickness of the substrate or a layer of the substrate. In some instances, the textures and/or patterns of the substrate may define at least part of an individually addressable location on the substrate. A textured and/or patterned substrate may be substantially planar. Alternatively, the substrate may be untextured and unpatterned.

A binder may be configured to immobilize an analyte or reagent to an individually addressable location. In some cases, a surface chemistry of an individually addressable location may comprise one or more binders. In some cases, a plurality of individually addressable locations may be coated with binders. The binders may be integral to the substrate. The binders may be added to the substrate. For instance, the binders may be added to the substrate as one or more coating layers. The substrate may comprise an order of magnitude of at least and/or at most about 10, 10², 10³, 10⁴, 10⁵, 10⁶, 10⁷, 10⁸, 10⁹, 10¹⁰, 10¹¹, 10¹², 10¹³ or more binders. The binders may immobilize analytes or reagents through non-specific interactions, such as one or more of hydrophilic interactions, hydrophobic interactions, electrostatic interactions, physical interactions (for instance, adhesion to pillars or settling within wells), and the like. Alternatively or in addition, the binders may immobilize analytes or reagents through specific interactions. For instance, where the analyte or reagent is a nucleic acid molecule, the binders may comprise oligonucleotide adapters configured to bind to the nucleic acid molecule. In other examples, the binders may comprise one or more of antibodies, oligonucleotides, nucleic acid molecules, aptamers, affinity binding proteins, lipids, carbohydrates, and the like. The binders may immobilize analytes or reagents through any possible combination of interactions. For instance, the binders may immobilize nucleic acid molecules through a combination of physical and chemical interactions, through a combination of protein and nucleic acid interactions, etc. A single binder may bind a single analyte or single reagent, a single binder may bind a plurality of analytes or a plurality of reagents, or a plurality of binders may bind a single analyte or a single reagent. In some instances, the substrate may comprise a plurality of types of binders, for example to bind different types of analytes or reagents. For example, a first type of binders (e.g., oligonucleotides) are configured to bind a first type of analyte (e.g., nucleic acid molecules) or reagent, and a second type of binders (e.g., antibodies) are configured to bind a second type of analyte (e.g., proteins) or reagent. In another example, a first type of binders (e.g., first type of oligonucleotide molecules) are configured to bind a first type of nucleic acid molecules and a second type of binders (e.g., second type of oligonucleotide molecules) are configured to bind a second type of nucleic acid molecules. For example, the substrate may be configured to bind different types of analytes or reagents in certain fractions or specific locations on the substrate by having the different types of binders in the certain fractions or specific locations on the substrate.

The substrate may be rotatable about an axis, referred to herein as a rotational axis. The rotational axis may or may not be an axis through the center of the substrate. The systems, devices, and apparatus described herein may further comprise an automated or manual rotational unit configured to rotate the substrate. The rotational unit may comprise a motor and/or a rotor. For instance, the substrate may be affixed to a chuck (such as a vacuum chuck). The substrate may be rotated at a rotational speed of at least about 1 revolution per minute (rpm), at least 2 rpm, at least 5 rpm, at least 10 rpm, at least 20 rpm, at least 50 rpm, at least 100 rpm, at least 200 rpm, at least 500 rpm, at least 1,000 rpm, at least 2,000 rpm, at least 5,000 rpm, at least 10,000 rpm, or greater. Alternatively or in addition, the substrate may be rotated at a rotational speed of at most about 10,000 rpm, 5,000 rpm, 2,000 rpm, 1,000 rpm, 500 rpm, 200 rpm, 100 rpm, 50 rpm, 20 rpm, 10 rpm, 5 rpm, 2 rpm, 1 rpm, or less. The substrate may be configured to rotate with different rotational velocities during different operations described herein, for example with higher velocities during reagent dispense and with lower velocities during analyte loading and imaging operations. The substrate may be configured to rotate with a rotational velocity that varies according to a time-dependent function, such as a ramp, sinusoid, pulse, or other function or combination of functions. The time-varying function may be periodic or aperiodic.

Analytes or reagents may be immobilized to the substrate during rotation. Analytes or reagents may be dispensed onto the substrate prior to or during rotation of the substrate. When the substrate is rotated at a relatively high rotational velocity, high speed coating across the substrate may be achieved via tangential inertia directing unconstrained spinning reagents in a partially radial direction (that is, away from the axis of rotation) during rotation, a phenomenon commonly referred to as centrifugal force. In some cases, the substrate may be rotated at relatively low velocities such that reagents dispensed to a certain location do not move to another location, or moves minimally, because of the rotation, to permit controlled dispensing of reagents to desired locations. For example, bead loading may be performed with controlled dispensing. For controlled dispensing, the substrate may rotate with a rotational frequency of no more than 60, 50, 40, 30, 25, 20, 15, 14, 13, 12, 11, 10, 9, 8, 7, 6, 5, 4, 3, 2, 1 rpm or less. In some cases the substrate may rotate with a rotational frequency of about 5 rpm during controlled dispensing. A speed of substrate rotation may be adjusted according to the appropriate operation (e.g., high speed for spin-coating, high speed for washing the substrate, low speed for sample loading, low speed for detection, low speed for analyte or reagent incubation, etc.).

In some cases, the substrate may be movable in any vector or direction. For example, such motion may be non-linear (e.g., in rotation about an axis), linear (e.g., on a rail track), or a hybrid of linear and non-linear motion. In some instances, the systems, devices, and apparatus described herein may further comprise a motion unit configured to move the substrate. The motion unit may comprise any mechanical component, such as a motor, rotor, actuator, linear stage, drum, roller, pulleys, etc., to move the substrate. Analytes or reagents may be immobilized to the substrate during any such motion. Analytes or reagents may be dispensed onto the substrate prior to, during, or subsequent to motion of the substrate.

Loading Reagents onto an Open Substrate

The surface of the substrate may be in fluid communication with at least one fluid nozzle (of a fluid channel). The surface may be in fluid communication with the fluid nozzle via a non-solid gap, e.g., an air gap. In some cases, the surface may additionally be in fluid communication with at least one fluid outlet. The surface may be in fluid communication with the fluid outlet via an air gap. The nozzle may be configured to direct a solution to the array. The outlet may be configured to receive a solution from the substrate surface. The solution may be directed to the surface using one or more dispensing nozzles. For example, the solution may be directed to the array using at least 1, 2, 3, 4, 5, 6, 7, 8, 9, 10, 11, 12, 13, 14, 15, 16, 17, 18, 19, 20 or more dispensing nozzles. In some cases, different reagents (e.g., nucleotide solutions of different types, different probes, washing solutions, etc.) may be dispensed via different nozzles, such as to prevent contamination. Each nozzle may be connected to a dedicated fluidic line or fluidic valve, which may further prevent contamination. Alternatively, some nozzles may share a fluidic line or fluidic valve, such as for pre-dispense mixing and/or to dispensing to multiple locations. A type of reagent may be dispensed via one or more nozzles. The one or more nozzles may be directed at or in proximity to a center of the substrate. Alternatively, the one or more nozzles may be directed at or in proximity to a location on the substrate other than the center of the substrate. Alternatively or in combination, one or more nozzles may be directed closer to the center of the substrate than one or more of the other nozzles. For instance, one or more nozzles used for dispensing washing reagents may be directed closer to the center of the substrate than one or more nozzles used for dispensing active reagents. The one or more nozzles may be arranged at different radii from the center of the substrate. The nozzles may be angled towards or away from a center of the substrate, or not angled (e.g., normal to the substrate plane). Two or more nozzles may be operated in combination to deliver fluids to the substrate more efficiently. One or more nozzles may be configured to deliver fluids to the substrate as a jet, spray (or other dispersed fluid), and/or droplets. One or more nozzles may be operated to nebulize fluids prior to delivery to the substrate. For example, the fluids may be delivered as aerosol particles.

In some cases, the solution may be dispensed on the substrate while the substrate is stationary; the substrate may then be subjected to rotation (or other motion) following the dispensing of the solution. Alternatively, the substrate may be subjected to rotation (or other motion) prior to the dispensing of the solution; the solution may then be dispensed on the substrate while the substrate is rotating (or otherwise moving). In some cases, rotation of the substrate may yield a centrifugal force (or inertial force directed away from the axis) on the solution, causing the solution to flow radially outward over the array. In this manner, rotation of the substrate may direct the solution across the array. Continued rotation of the substrate over a period of time may dispense a fluid film of a nearly constant thickness across the array.

One or more conditions such as the rotational velocity of the substrate, the acceleration of the substrate (e.g., the rate of change of velocity), viscosity of the solution, angle of dispensing (e.g., contact angle of a stream of reagents) of the solution, radial coordinates of dispensing of the solution (e.g., on center, off center, etc.), temperature of the substrate, temperature of the solution, and other factors may be adjusted and/or otherwise optimized to attain a desired wetting on the substrate and/or a film thickness on the substrate, such as to facilitate uniform coating of the substrate. For instance, one or more conditions may be applied to attain a film thickness of at least and/or at most about 10 nanometers (nm), 20 nm, 50 nm, 100 nm, 200 nm, 500 nm, 1 μm, 2 μm, 5 μm, 10 μm, 20 μm, 50 μm, 100 μm, 200 μm, 500 μm, 1 mm, or more. One or more conditions may be applied to attain a film thickness that is within a range defined by any two of the preceding values. In some cases, a surfactant may be added to the solution, or a surfactant may be added to the surface to facilitate uniform coating or to facilitate sample loading efficiency. Alternatively or in conjunction, the thickness of the solution may be adjusted using mechanical, electric, physical, or other mechanisms. For example, the solution may be dispensed onto a substrate and subsequently leveled using, e.g., a physical scraper such as a squeegee, to obtain a desired thickness of uniformity across the substrate.

Reagents may be dispensed to the substrate to multiple locations, and/or multiple reagents may be dispensed to the substrate to a single location, via different mechanisms. Reagent dispensing mechanisms disclosed herein may be applicable to sample dispensing. For example, a reagent may comprise the sample. The term “loading onto a substrate,” as used herein, may refer to dispensing of the reagent or the sample to a surface of the substrate in accordance with any reagent dispensing mechanism described herein.

In some cases, dispensing may be achieved via relative motion of the substrate and the dispenser (e.g., nozzle). For example, a reagent may be dispensed to the substrate at a first location, and thereafter travel to a second location different from the first location due to forces (e.g., centrifugal forces, centripetal forces, inertial forces, etc.) caused by motion of the substrate (e.g., rotational motion of the substrate, linear motion of the substrate, combination thereof, etc.). In another example, a reagent may be dispensed to a reference location, and the substrate may be moved relative to the reference location such that the reagent is dispensed to multiple locations of the substrate. In another example, a dispenser may be moved relative to the substrate to dispense the reagent at different locations, for example moved prior to, during, or subsequent to dispensing. In an example, a reagent is ‘painted’ onto the substrate by moving the dispenser and/or the substrate relative to each other, along a desired path on the substrate. The open substrate geometry may allow for flexible and controlled dispensing of a reagent to a desired location on the substrate. In some cases, dispensing may be achieved without relative motion between the substrate and the dispenser. For example, multiple dispensers may be used to dispense reagents to different locations, and/or multiple reagents to a single location, or a combination thereof (e.g., multiple reagents to multiple locations).

In another example, an external force (e.g., involving a pressure differential, involving physical force, involving a magnetic force, involving an electrical force, etc.), such as wind, a field-generating device, or a physical device, may be applied to one or more surfaces of the substrate to direct reagents to different locations across the substrate. In another example, the method for dispensing reagents may comprise vibration. In such an example, reagents may be distributed or dispensed onto a single region or multiple regions of the substrate. The substrate may then be subjected to vibration, which may spread the reagent to different locations across the substrate. Alternatively or in conjunction, the method may comprise using mechanical, electric, physical, or other mechanisms to dispense reagents to the substrate. For example, the solution may be dispensed onto a substrate and a physical scraper (e.g., a squeegee) may be used to spread the dispensed material or spread the reagents to different locations and/or to obtain a desired thickness or uniformity across the substrate. Beneficially, such flexible dispensing may be achieved without contamination of the reagents.

In some instances, where a volume of reagent is dispensed to the substrate at a first location, and thereafter travels to a second location different from the first location, the volume of reagent may travel in a path or paths, such that the travel path or paths are coated with the reagent. In some cases, such travel path or paths may encompass a desired surface area (e.g., entire surface area, partial surface area(s), etc.) of the substrate. In some instances, two or more reagents may be mixed on the surface of the substrate, such as by being dispensed at the same location and/or by directing a first reagent to travel to meet additional reagent(s). In some instances, the mixture of reagents formed on the substrate may be homogenous or substantially homogenous. The mixture of reagents may be formed at a first location on the substrate prior to dispersing the mixing of reagents to other locations on the substrate, such as at locations to meet other reagents or analytes.

In some embodiments, one or more solutions may be delivered directly to the reaction site without substantial displacement of the one or more solution from the point of delivery. Methods of direct delivery of a solution to the reaction site may include aerosol delivery of the solution, applying the solution using an applicator, curtain-coating the solution, slot-die coating, dispensing the solution from a translating dispense probe, dispensing the solution from an array of dispense probes, dipping the substrate into the solution, or contacting the substrate to a sheet comprising the solution.

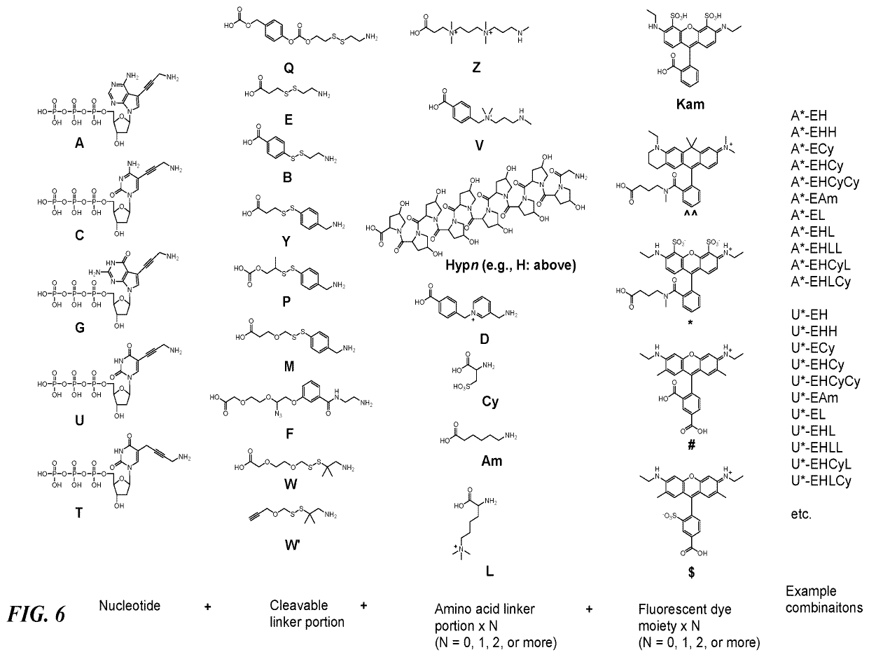

Aerosol delivery may comprise delivering a solution to the substrate in aerosol form by directing the solution to the substrate using a pressure nozzle or an ultrasonic nozzle. Applying the solution using an applicator may comprise contacting the substrate with an applicator comprising the solution and translating the applicator relative to the substrate. For example, applying the solution using an applicator may comprise painting the substrate. The solution may be applied in a pattern by translating the applicator, rotating the substrate, translating the substrate, or a combination thereof. Curtain-coating may comprise dispensing the solution from a dispense probe to the substrate in a continuous stream (e.g., a curtain or a flat sheet) and translating the dispense probe relative to the substrate. A solution may be curtain-coated in a pattern by translating the dispense probe, rotating the substrate, translating the substrate, or a combination thereof. Slot-die coating may comprise dispensing the solution from a dispense probe positioned near the substrate such that the solution forms a meniscus between the substrate and the dispense probe and translating the dispense probe relative to the substrate. A solution may be slot-die coated in a pattern by translating the dispense probe, rotating the substrate, translating the substrate, or a combination thereof. Dispensing the solution from a translating dispense probe may comprise translating the dispense probe relative to the substrate in a pattern (e.g., a spiral pattern, a circular pattern, a linear pattern, a striped pattern, a cross-hatched pattern, or a diagonal pattern). Dispensing the solution from an array of dispense probes may comprise dispensing the solution from an array of nozzles (e.g., a shower head) positioned above the substrate such that the solution is dispensed across an area of the substrate substantially simultaneously. Dipping the substrate into the solution may comprise dipping the substrate into a reservoir comprising the solution. In some embodiments, the reservoir may be a shallow reservoir to reduce the volume of the solution required to coat the substrate. Contacting the substrate to a sheet comprising the solution may comprise bringing the substrate in contact with a sheet of material (e.g., a porous sheet or a fibrous sheet) permeated with the solution. The solution may be transferred to the substrate. In some embodiments, the sheet of material may be a single-use sheet. In some embodiments, the sheet of material may be a reusable sheet. In some embodiments, a solution may be dispensed onto a substrate using the method illustrated in FIG. 5B, where a jet of a solution may be dispensed from a nozzle to a rotating substrate. The nozzle may translate radially relative to the rotating substrate, thereby dispensing the solution in a spiral pattern onto the substrate.

One or more solutions or reagents may be delivered to a substrate by any of the delivery methods disclosed herein. Two or more solutions or reagents may be delivered to the substrate using the same or different delivery methods. Two or more solutions may be delivered to the substrate such that the time between contacting a solution or reagent and a subsequent solution or reagent is substantially similar for each region of the substrate contacted to the one or more solutions or reagents. A solution or reagent may be delivered as a single mixture. The solution or reagent may be dispensed in two or more component solutions. For example, each component of the two or more component solutions may be dispensed from a distinct nozzle. The distinct nozzles may dispense the two or more component solutions substantially simultaneously to substantially the same region of the substrate such that a homogenous solution forms on the substrate. Dispensing of each component of the two or more components may be temporally separated. Dispensing of each component may be performed using the same or different delivery methods. Direct delivery of a solution or reagent may be combined with spin-coating.

A solution may be incubated on the substrate for any desired duration (e.g., minutes, hours, etc.). In some embodiments, the solution may be incubated on the substrate under conditions that maintain a layer of fluid on the surface. One or more of the temperature of the chamber, the humidity of the chamber, the rotation of the substrate, and the composition of the fluid may be adjusted such that the layer of fluid is maintained during incubation. In some instances, during incubation, the substrate may be rotated at an rotational frequency of no more than 60 rpm, 50 rpm, 40 rpm, 30 rpm, 25 rpm, 20 rpm, 15 rpm, 14 rpm, 13 rpm, 12 rpm, 11 rpm, 10 rpm, 9 rpm, 8 rpm, 7 rpm, 6 rpm, 5 rpm, 4 rpm, 3 rpm, 2 rpm, 1 rpm or less. In some cases, the substrate may rotate with a rotational frequency of about 5 rpm during incubation.

The substrate or a surface thereof may comprise other features that aid in solution or reagent retention on the substrate or thickness uniformity of the solution or reagent on the substrate. In some cases, the surface may comprise a raised edge (e.g., a rim) which may be used to retain solution on the surface. The surface may comprise a rim near the outer edge of the surface, thereby reducing the amount of the solution that flows over the outer edge.

The dispensed solution may comprise any sample or any analyte disclosed herein. The dispensed solution may comprise any reagent disclosed herein. In some cases, the solution may be a reaction mixture comprising a variety of components. In some cases, the solution may be a component of a final mixture (e.g., to be mixed after dispensing). In non-limiting examples, the solution can comprise samples, analytes, supports, beads, probes, nucleotides, oligonucleotides, labels (e.g., dyes), terminators (e.g., blocking groups), other components to aid, accelerate, or decelerate a reaction (e.g., enzymes, catalysts, buffers, saline solutions, chelating agents, reducing agents, other agents, etc.), washing solution, cleavage agents, combinations thereof, deionized water, and other reagents and buffers.

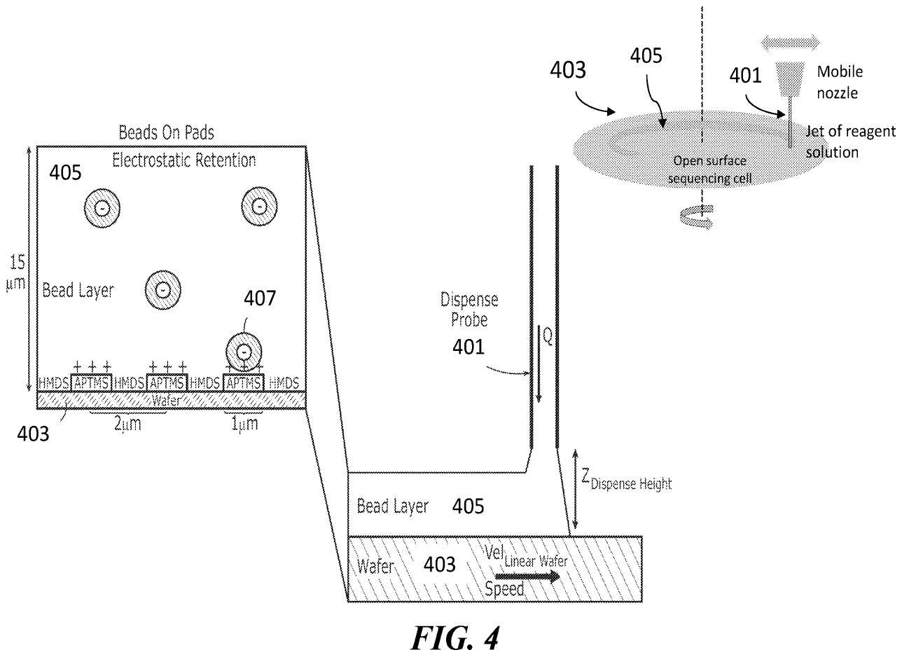

A sample may comprise beads, as described elsewhere herein, for example beads comprising nucleic acid colonies bound thereto. In some cases, an order of magnitude of at least and/or at most about 10¹, 10², 10³, 10⁴, 10⁵, 10⁶, 10⁷, 10⁸, 10⁹, 10¹⁰, 10¹¹, 10¹², 10¹³ or more beads may be loaded on the substrate, such as to immobilize to as many individually addressable locations. In some cases, the beads may be distinguishable from one another using a property of the beads, such as color, reflectance, anisotropy, brightness, fluorescence, etc. In some cases, as described elsewhere herein, different beads may comprise different tags (e.g., nucleic acid sequences) coupled thereto. For example, a bead may comprise an oligonucleotide molecule comprising a tag (e.g., barcode) that identifies a bead amongst a plurality of beads. FIG. 3 illustrates images of a portion of a substrate surface after loading a sample containing beads onto a substrate patterned with a substantially hexagonal lattice of individually addressable locations, where the right panel illustrates a zoomed-out image of a portion of a surface, and the left panel illustrates a zoomed-in image of a section of the portion of the surface. After sample loading, a “bead occupancy” may generally refer to the number of a type of individually addressable locations comprising at least one bead out of the total number of individually addressable locations of the same type. A bead “landing efficiency” may generally refer to the number of beads that bind to the surface out of the total number of beads dispensed on the surface.

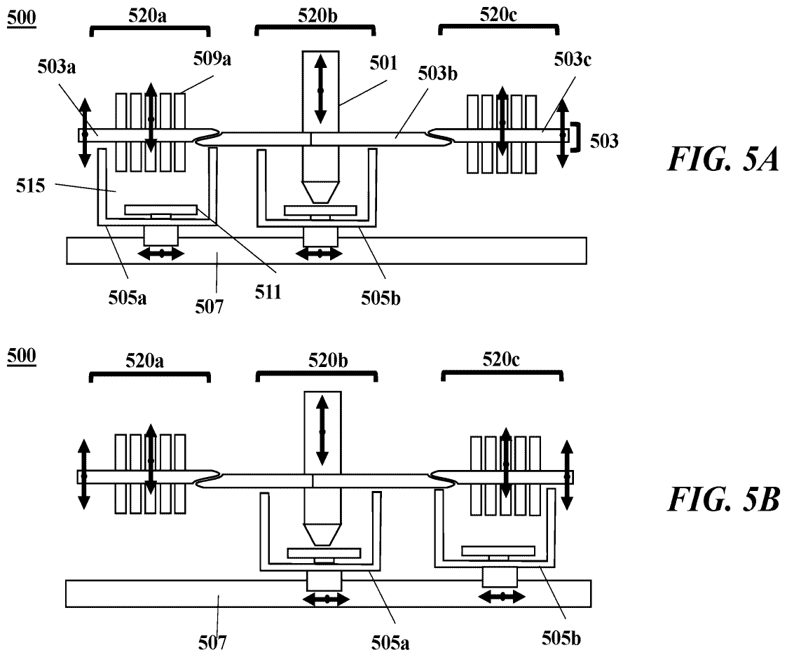

In some cases, beads may be dispensed to the substrate according to one or more systems and methods shown in FIG. 4. As shown in FIG. 4, a solution comprising beads may be dispensed from a dispense probe 401 (e.g., a nozzle) to a substrate 403 (e.g., a wafer) to form a layer 405. The dispense probe may be positioned at a height (“Z”) above the substrate. In the illustrated example, the beads are retained in the layer 405 by electrostatic retention and may immobilize to the substrate at respective individually addressable locations. A set of beads in the solution may each comprise a population of amplified products (e.g., nucleic acid molecules) immobilized thereto, which amplified products accumulate to a negative charge on the bead. Otherwise, the beads may comprise reagents that have a negative charge. The substrate comprises alternating surface chemistry between distinguishable locations, in which a first location type comprises APTMS carrying a positive charge with affinity towards the negative charge of the amplified bead (e.g., a bead comprising amplified products immobilized thereto, and as distinguished from a negative bead which does not the comprise the same) or other bead comprising the negative charge, and a second location type comprises HMDS which has lower affinity and/or is repellant of the amplified bead or other bead comprising the negative charge. Within the layer 405, a bead may successfully land on a first location of the first location type (as in 407). In the illustrated example, the location size is 1 micron, the pitch between the different locations of the same location type (e.g., first location type) is 2 microns, and the layer has a depth of 15 micron. The top right panel illustrates that a reagent solution may be dispensed from the dispense probe 401 as the layer 405 along a path on an open surface of the substrate 403. The reagent may be dispensed on the surface in any desired pattern or path. The substrate 403 and the dispense probe 401 may move in any configuration with respect to each other to achieve any pattern (e.g., linear pattern, substantially spiral pattern, etc.).

Dispense mechanisms described herein may be operated by a fluid flow unit which may be controlled by one or more controllers, individually or collectively. The fluid flow unit may comprise any of the hardware and software components described with respect to the dispense mechanisms herein.

### Detection on Open Substrate

An optical system comprising a detector may be configured to detect one or more signals from a detection area on the substrate prior to, during, or subsequent to, the dispensing of reagents to generate an output. Signals from multiple individually addressable locations may be detected during a single detection event. Signals from the same individually addressable location may be detected in multiple instances.

A signal may be an optical signal (e.g., fluorescent signal), electronic signal, or any detectable signal. The signal may be detected during rotation of the substrate or following termination of the rotation. The signal may be detected while the analyte is in fluid contact with a solution. The signal may be detected following washing of the solution. In some instances, after the detection, the signal may be muted, such as by cleaving a label from a probe and/or the analyte, and/or modifying the probe and/or the analyte. Such cleaving and/or modification may be performed by one or more stimuli, such as exposure to a chemical, an enzyme, light (e.g., ultraviolet light), or temperature change (e.g., heat). In some instances, the signal may otherwise become undetectable by deactivating or changing the mode (e.g., detection wavelength) of the one or more sensors, or terminating or reversing an excitation of the signal. In some instances, detection of a signal may comprise capturing an image or generating a digital output (e.g., between different images).

The operations of (i) directing a solution to the substrate and (ii) detection of one or more signals indicative of a reaction between a probe in the solution and an analyte immobilized to the substrate, may be repeated any number of times. Such operations may be repeated in an iterative manner. For example, the same analyte immobilized to a given location in the array may interact with multiple solutions in multiple cycles and for each iteration, the additional signals detected may provide incremental, or final, data about the analyte during the processing. For example, where the analyte is a nucleic acid molecule and the processing is sequencing, additional signals detected for each iteration may be indicative of one or more bases in the nucleic acid sequence of the nucleic acid molecule. In some cases, multiple solutions can be provided to the substrate without intervening detection events. In some cases, multiple detection events can be performed after a single flow of solution. In some instances, a washing solution, cleaving solution (e.g., comprising cleavage agent), and/or other solutions may be directed to the substrate between each operation, between each cycle, or a certain number of times for each cycle.

The optical system may be configured for continuous area scanning of a substrate during rotational motion of the substrate. The term “continuous area scanning (CAS),” as used herein, generally refers to a method in which an object in relative motion is imaged by repeatedly (e.g., electronically or computationally) advancing (clocking or triggering) an array sensor at a velocity that compensates for object motion in the detection plane (focal plane). CAS can produce images having a scan dimension larger than the field of the optical system. TDI scanning may be an example of CAS in which the clocking entails shifting photoelectric charge on an area sensor during signal integration. For a TDI sensor, at each clocking step, charge may be shifted by one row, with the last row being read out and digitized. Other modalities may accomplish similar function by high-speed area imaging and co-addition of digital data to synthesize a continuous or stepwise continuous scan.

The optical system may comprise one or more sensors. The sensors may detect an image optically projected from the sample. The optical system may comprise one or more optical elements. An optical element may be, for example, a lens, tube lens, prism, mirror, wave plate, filter, attenuator, grating, diaphragm, beam splitter, diffuser, polarizer, depolarizer, retroreflector, spatial light modulator, or any other optical element. The system may comprise any number of sensors. In some cases, a sensor is any detector as described herein. In some examples, the sensor may comprise image sensors, CCD cameras, CMOS cameras, TDI cameras (e.g., TDI line-scan cameras), pseudo-TDI rapid frame rate sensors, or CMOS TDI or hybrid cameras. The optical system may further comprise any one or more optical sources (e.g., lasers, LED light sources, etc.). In some cases, where there are multiple sensors, the different sensors may image the same or different regions of the rotating substrate, in some cases simultaneously. Each sensor of the plurality of sensors may be clocked at a rate appropriate for the region of the rotating substrate imaged by the sensor, which may be based on the distance of the region from the center of the rotating substrate or the tangential velocity of the region. In some cases, multiple scan heads can be operated in parallel along different imaging paths (e.g., interleaved spiral scans, nested spiral scans, interleaved ring scans, nested ring scans). A scan head may comprise one or more of a detector element such as a camera (e.g., a TDI line-scan camera), an illumination source (e.g., as described herein), and one or more optical elements (e.g., as described herein).

The system may further comprise one or more controllers operatively coupled to the one or more sensors, individually or collectively programmed to process optical signals from the one or more sensors, such as for each region of the rotating substrate.

In some cases, the optical system may comprise an immersion objective lens. The immersion objective lens may be in contact with an immersion fluid that is in contact with the open substrate. The immersion fluid may comprise any suitable immersion medium for imaging (e.g., water, aqueous, organic solution). In some cases, an enclosure may partially or completely surround a sample-facing end of the optical imaging objective. The enclosure may be configured to contain the immersion fluid. The enclosure may not be in contact with the substrate; for example, a gap between the enclosure and the substrate may be filled by the fluid contained by the enclosure (e.g., the enclosure can retain the fluid via surface tension). In some cases, an electric field may be used to regulate a hydrophobicity of one or more surfaces of the container to retain at least a portion of the fluid contacting the immersion objective lens and the open substrate. In some cases, the immersion fluid may be continuously replenished or recycled via an inlet and outlet to the enclosure.

### High Throughput Substrate Processing

An open substrate may be processed within a modular local sample processing environment. One or more surfaces of the substrate may be exposed to and accessible from a surrounding open environment (e.g., a sample processing environment). In some cases, the surrounding open environment may be controlled and/or confined in a larger controlled environment. A barrier comprising a fluid barrier may be maintained between a sample processing environment and an exterior environment during certain processing operations, such as reagent dispensing and detecting. Systems and methods comprising a fluid barrier are described in further detail in U.S. Patent Pub. No. 20210354126A1, which is entirely incorporated herein by reference. A modular local sample processing environment may be defined by a chamber and a lid plate, where the lid plate is not in contact with the chamber, and the gap between the lid plate and the chamber may comprise the fluid barrier. The fluid barrier may comprise fluid (e.g., air) from the sample processing environment and/or the exterior environment and may have lower pressure than the sample processing environment, the external environment, or both. The fluid in the fluid barrier may be in coherent motion or bulk motion.

The sample processing environment may comprise therein a substrate, such as any substrate described elsewhere herein. Any operation performed on or with the substrate, as described elsewhere herein, may be performed within the sample processing environment while the fluid barrier is maintained. For example, the substrate may be rotated within the sample processing environment during various operations. In another example, fluid may be directed to the substrate while the substrate is in the sample processing environment, via a fluid handler (e.g., nozzle) that penetrates the lid plate into the sample processing environment. In another example, a detector can image the substrate while the substrate is in the sample processing environment, via a detector that penetrates the lid plate into the sample processing environment. Beneficially, the fluid barrier may help maintain temperature(s) and/or relative humidit(ies), or ranges thereof, within the sample processing environment during various processing operations.

The systems described herein, or any element thereof, may be environmentally controlled. For instance, the systems may be maintained at a specified temperature or humidity. For an operation, the systems (or any element thereof) may be maintained at a temperature of at least and/or at most 20 degrees Celsius (° C.), 25° C., 30° C., 35° C., 40° C., 45° C., 50° C., 55° C., 60° C., 65° C., 70° C., 75° C., 80° C., 85° C., 90° C., 95° C., 100° C., or more. Different elements of the system may be maintained at different temperatures or within different temperature ranges, such as the temperatures or temperature ranges described herein. Elements of the system may be set at temperatures above the dew point to prevent condensation. Elements of the system may be set at temperatures below the dew point to collect condensation.

While examples described herein provide relative rotational motion of the substrates and/or detector systems, the substrates and/or detector systems may alternatively or additionally undergo relative non-rotational motion, such as relative linear motion, relative non-linear motion (e.g., curved, arcuate, angled, etc.), and any other types of relative motion.

An open substrate may be retained in the same or approximately the same physical location during processing of an analyte and subsequent detection of a signal associated with the processed analyte. Alternatively, different operations on or with the open substrate may be performed in different stations disposed in different physical locations. For example, a first station may be disposed above, below, adjacent to, or across from a second station. In some cases, the different stations can be housed within an integrated housing. Alternatively, the different stations can be housed separately. In some cases, different stations may be separated by a barrier, such as a retractable barrier (e.g., sliding door). One or more different stations of a system, or portions thereof, may be subjected to different physical conditions, such as different temperatures, pressures, or atmospheric compositions. The open substrate may transition between different stations by transporting the sample processing environment comprising the chamber containing the open substrate between the different stations. One or more mechanical components or mechanisms, such as a robotic arm, elevator mechanism, actuators, rails, and the like, or other mechanisms may be used to transport the sample processing environment.

An environmental unit (e.g., humidifiers, heaters, heat exchangers, compressors, etc.) may be configured to regulate one or more operating conditions in each station. In some instances, independent environmental units may regulate each station. In some instances, a single environmental unit may regulate a plurality of stations. In some instances, a plurality of environmental units may, individually or collectively, regulate the different stations. An environmental unit may use active methods or passive methods to regulate the operating conditions. For example, the temperature may be controlled using heating or cooling elements. The humidity may be controlled using humidifiers or dehumidifiers. In some instances, a part of a particular station, such as within a sample processing environment, may be further controlled from other parts of the particular station. Different parts may have different local temperatures, pressures, and/or humidity. In one example, the delivery and/or dispersal of reagents may be performed in a first station having a first operating condition, and the detection process may be performed in a second station having a second operating condition different from the first operating condition. The first station may be at a first physical location in which the open substrate is accessible to a fluid handling unit during the delivery and/or dispersal processes, and the second station may be at a second physical location in which the open substrate is accessible to the detector system.

One or more modular sample environment systems (each having its own barrier system, e.g., fluid barrier) can be used between the different stations. In some instances, the systems described herein may be scaled up to include two or more of a same station type. For example, a sequencing system may include multiple processing and/or detection stations. FIGS. 5A-5B illustrate a system 500 that multiplexes two modular sample environment systems in a three-station system.

In FIG. 5A, a first chemistry station (e.g., 520a) can operate (e.g., dispense reagents, e.g., to incorporate nucleotides to perform sequencing by synthesis) via at least a first operating unit (e.g., fluid dispenser 509a) on a first substrate (e.g., 511) in a first sample environment system (e.g., 505a) while substantially simultaneously, a detection station (e.g., 520b) can operate (e.g., scan) on a second substrate in a second sample environment system (e.g., 505b) via at least a second operating unit (e.g., detector 501), while substantially simultaneously, a second chemistry station (e.g., 520c) sits idle. An idle station may not operate on a substrate. An idle station (e.g., 520c) may be recharged, reloaded, replaced, cleaned, washed (e.g., to flush reagents), calibrated, reset, kept active (e.g., power on), and/or otherwise maintained during an idle time. After an operating cycle is complete, the sample environment systems may be re-stationed, as in FIG. 5B, where the second substrate in the second sample environment system (e.g., 505b) is re-stationed from the detection station (e.g., 520b) to the second chemistry station (e.g., 520c) for operation (e.g., dispensing of reagents, e.g., to incorporate nucleotides to perform sequencing by synthesis) by the second chemistry station, and the first substrate in the first sample environment system (e.g., 505a) is re-stationed from the first chemistry station (e.g., 520a) to the detection station (e.g., 520b) for operation (e.g., scanning) by the detection station. An operating cycle may be deemed complete when operation at each active, parallel station is complete. During re-stationing, the different sample environment systems may be physically moved (e.g., along the same track or dedicated tracks, e.g., rail(s) 507) to the different stations and/or the different stations may be physically moved to the different sample environment systems. One or more components of a station, such as modular plates 503a, 503b, 503c of plate 503 (e.g., lid plate) defining a particular station(s), may be physically moved to allow a sample environment system to exit the station, enter the station, or cross through the station. During processing of a substrate at station, the environment of a sample environment region (e.g., 515) of a sample environment system (e.g., 505a) may be controlled and/or regulated according to the station's requirements. After the next operating cycle is complete, the sample environment systems can be re-stationed again, such as back to the configuration of FIG. 5A, and this re-stationing can be repeated (e.g., between the configurations of FIGS. 5A and 5B) with each completion of an operating cycle until the required processing for a substrate is completed. In this illustrative re-stationing scheme, the detection station may be kept active (e.g., not have idle time not operating on a substrate) for all operating cycles by providing alternating different sample environment systems to the detection station for each consecutive operating cycle. Beneficially, use of the detection station is optimized. Given different processing or equipment needs, an operator may opt to run the two chemistry stations substantially simultaneously while the detection station is kept idle.

Beneficially, different operations within the system may be multiplexed with high flexibility and control. For example, as described herein, one or more processing stations may be operated in parallel with one or more detection stations on different substrates in different modular sample environment systems to reduce or eliminate lag between different sequences of operations (e.g., chemistry first, then detection). The modular sample environment systems may be translated between the different stations accordingly to optimize efficient equipment use (e.g., such that the detection station is in operation almost 100% of the time). In some examples, at least 1, 2, 3, 4, 5, 6, 7, 8, 9, or more modules or stations of the sequencing system may be multiplexed. For example, 2 or more of the modules may each perform their intended function simultaneously or according to the methods described elsewhere herein. An example of this may comprise two-station multiplexing of an optics station and a chemistry station as described herein. Another example may comprise multiplexing three or more stations and process phases. For example, the method may comprise using staggered chemistry phases sharing a scanning station. The scanning station may be a high-speed scanning station. The modules or stations may be multiplexed using various sequences and configurations.

The nucleic acid sequencing systems and optical systems described herein (or any elements thereof) may be combined in a variety of architectures.

### Reagent Components

A substrate may comprise a wafer or a support. A substrate may comprise an object acted upon by an enzyme and/or polymerase (e.g., a labeled nucleotide or other reagent). Thus, in some cases, a substrate (e.g., a labeled reagent) may be placed (e.g., loaded) on a substrate (e.g., a wafer). A labeled reagent may comprise a labeled reagent or labeled object.

### Optical Moieties

An optical moiety, or fluorescent moiety, may also be referred to herein as a “label.” An optical moiety generally refers to a detectable moiety that emits a signal (or reduces an already emitted signal) that can be detected. The label may be luminescent (e.g., fluorescent or phosphorescent). For example, the label may be or comprise a fluorescent moiety (e.g., a dye).

Non-limiting examples of dyes include SYBR green, SYBR blue, DAPI, propidium iodine, Hoechst, SYBR gold, ethidium bromide, acridine, proflavine, acridine orange, acriflavine, fluorocoumarin, ellipticine, daunomycin, chloroquine, distamycin D, chromomycin, homidium, mithramycin, ruthenium polypyridyls, anthramycin, phenanthridines and acridines, propidium iodide, hexidium iodide, dihydroethidium, ethidium homodimer-1 and -2, ethidium monoazide, ACMA, Hoechst 33258, Hoechst 33342, Hoechst 34580, DAPI, acridine orange, 7-AAD, actinomycin D, LDS751, hydroxystilbamidine, SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO labels (e.g., SYTO-40, -41, -42, -43, -44, and -45 (blue); SYTO-13, -16, -24, -21, -23, -12, -11, -20, -22, -15, -14, and -25 (green); SYTO-81, -80, -82, -83, -84, and -85 (orange); and SYTO-64, -17, -59, -61, -62, -60, and -63 (red)), fluorescein, fluorescein isothiocyanate (FITC), tetramethyl rhodamine isothiocyanate (TRITC), rhodamine, tetramethyl rhodamine, R-phycoerythrin, Cy-2, Cy-3, Cy-3.5, Cy-5, Cy5.5, Cy-7, Texas Red, Phar-Red, allophycocyanin (APC), Sybr Green I, Sybr Green II, Sybr Gold, CellTracker Green, 7-AAD, ethidium homodimer I, ethidium homodimer II, ethidium homodimer III, ethidium bromide, umbelliferone, eosin, green fluorescent protein, erythrosin, coumarin, methyl coumarin, pyrene, malachite green, stilbene, lucifer yellow, cascade blue, dichlorotriazinylamine fluorescein, dansyl chloride, fluorescent lanthanide complexes such as those including europium and terbium, carboxy tetrachloro fluorescein, 5 and/or 6-carboxy fluorescein (FAM), VIC, 5- (or 6-) iodoacetamidofluorescein, 5-{[2(and 3)-5-(Acetylmercapto)-succinyl]amino}fluorescein (SAMSA-fluorescein), lissamine rhodamine B sulfonyl chloride, 5 and/or 6 carboxy rhodamine (ROX), 7-amino-methyl-coumarin, 7-Amino-4-methylcoumarin-3-acetic acid (AMCA), BODIPY fluorophores, 8-methoxypyrene-1,3,6-trisulfonic acid trisodium salt, 3,6-Disulfonate-4-amino-naphthalimide, phycobiliproteins, AlexaFluor labels (e.g., AlexaFluor 350, 405, 430, 488, 532, 546, 555, 568, 594, 610, 633, 635, 647, 660, 680, 700, 750, and 790 dyes), DyLight labels (e.g., DyLight 350, 405, 488, 550, 594, 633, 650, 680, 755, and 800 dyes), Black Hole Quencher Dyes (Biosearch Technologies) (e.g., BH1-0, BHQ-1, BHQ-3, and BHQ-10), QSY Dye fluorescent quenchers (Molecular Probes/Invitrogen) (e.g., QSY7, QSY9, QSY21, and QSY35), Dabcyl, Dabsyl, Cy5Q, Cy7Q, Dark Cyanine dyes (GE Healthcare), Dy-Quenchers (Dyomics) (e.g., DYQ-660 and DYQ-661), ATTO fluorescent quenchers (ATTO-TEC GmbH) (e.g., ATTO 540Q, ATTO 580Q, ATTO 612Q, Atto532 [e.g., ATTO 532 succinimidyl ester], and Atto633), Kam, and other fluorophores and/or quenchers.

A fluorescent dye may be excited (e.g., have an excitation maximum in a region of the electromagnetic spectrum) by the application of energy corresponding to the visible region of the electromagnetic spectrum (e.g., between about 430-770 nm). Excitation may be done using any useful apparatus, such as a laser and/or light emitting diode. A fluorescent dye may be excited over a single wavelength or a range of wavelengths. A fluorescent dye may be excitable by light in the red region of the visible portion of the electromagnetic spectrum (about 625-740 nm). Alternatively or additionally, fluorescent dye may be excitable by light in the green region of the visible portion of the electromagnetic spectrum (about 500-565 nm).

A fluorescent dye may emit light (e.g., fluorescence) in the visible region of the electromagnetic spectrum (e.g., between about 430-770 nm) (e.g., may have an emission maximum in the red region of the visible portion of the electromagnetic spectrum). A fluorescent dye may emit signal in the red region of the visible portion of the electromagnetic spectrum (about 625-740 nm). Alternatively or additionally, fluorescent dye may emit signal in the green region of the visible portion of the electromagnetic spectrum (about 500-565 nm).

A label may be a quencher. The term “quencher,” as used herein, generally refers to molecules that may be energy acceptors (e.g., a molecule that can reduce an emitted signal). Luminescence and/or fluorescence from labels may be quenched. In some cases, the label may be a type that does not self-quench or exhibit proximity quenching. Non-limiting examples of a label type that does not self-quench or exhibit proximity quenching include Bimane derivatives such as Monobromobimane. The term “proximity quenching,” as used herein, generally refers to a phenomenon where one or more dyes near each other may exhibit lower fluorescence as compared to the fluorescence they exhibit individually. In some cases, the dye may be subject to proximity quenching wherein the donor dye and acceptor dye are within 1 nm to 50 nm of each other. Examples of quenchers include, but are not limited to, Black Hole Quencher Dyes (Biosearch Technologies) (e.g., BH1-0, BHQ-1, BHQ-3, and BHQ-10), QSY Dye fluorescent quenchers (Molecular Probes/Invitrogen) (e.g., QSY7, QSY9, QSY21, and QSY35), Dabcyl, Dabsyl, Cy5Q, Cy7Q, Dark Cyanine dyes (GE Healthcare), Dy-Quenchers (Dyomics) (e.g., DYQ-660 and DYQ-661), and ATTO fluorescent quenchers (ATTO-TEC GmbH) (e.g., ATTO 540Q, ATTO 580Q, and ATTO 612Q). Fluorophore donor molecules may be used in conjunction with a quencher. Examples of fluorophore donor molecules that can be used in conjunction with quenchers include, but are not limited to, fluorophores such as Cy3B, Cy3, or Cy5; Dy-Quenchers (Dyomics) (e.g., DYQ-660 and DYQ-661); and ATTO fluorescent quenchers (ATTO-TEC GmbH) (e.g., ATTO 540Q, 580Q, and 612Q).

### Linkers

An association between a linker and a substrate can be any suitable association including a covalent or non-covalent bond. For example, a linker may be coupled to an object (e.g., nucleotide) via a nucleobase of a nucleotide via, e.g., a propargyl or propargylamino moiety. In another example, a linker may be coupled to an object (e.g., protein, such as an antibody) via an amino acid of a polypeptide or protein. In some cases, an association between a linker and an object may be a biotin-avidin interaction. In other cases, an association between a linker and an object may be via a propargylamino moiety. In some cases, an association between a linker and an object may be via an amide bond (e.g., a peptide bond). A linker may comprise a cleavable moiety configured to be cleaved to separate the labeling reagent or a portion thereof from an object to which it is attached.

A linker may comprise an amino acid. A linker may comprise at least and/or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80 amino acids. A linker may comprise a plurality of different types of amino acids. An amino acid may be proteinogenic or non-proteinogenic. A “proteinogenic amino acid,” as used herein, generally refers to a genetically encoded amino acid that may be incorporated into a protein during translation. Proteinogenic amino acids include arginine, histidine, lysine, aspartic acid, glutamic acid, serine, threonine, asparagine, glutamine, cysteine, selenocysteine, glycine, proline, alanine, isoleucine, leucine, methionine, phenylalanine, tryptophan, tyrosine, valine, selenocysteine, and pyrrolysine. A “non-proteinogenic amino acid,” as used herein, is an amino acid that is not a proteinogenic amino acid. A non-proteinogenic amino acid may be a naturally occurring amino acid or a non-naturally occurring amino acid. Non-proteinogenic amino acids include amino acids that are not found in proteins and/or are not naturally encoded or found in the genetic code of an organism. Examples of non-proteinogenic amino acid include, but are not limited to, (all-S,all-E)-3-amino-9-methoxy-2,6,8-trimethyl-10-phenyldeca-4,6-dienoic acid (ADDA), 2-aminoisobutyric acid, ay-aminobutyric acid, 4-aminobenzoic acid, 4-hydroxyphenylglycine, 6-aminohexanoic acid, aminolevulinic acid, δ-aminolevulinic acid, azetidine-2-carboxylic acid, alloisoleucine, allothreonine, canaline, canavanine, carboxyglutamic acid, chloroalanine, citrulline, cysteic acid, 5-amino-5-carboxy-N,N,N-trimethylpentan-1-aminium (also known as 2-amino-itr6-(trimethylammonio)hexanoate), dehydroalanine, diaminopimelic acid, dihydroxyphenylglycine, enduracididine, gamma-aminobutyric acid, hawkinsin, homocysteine, homoserine, hydroxyproline, hypusine, isovaline, isoserine, lanthionine, t-leucine, norleucine, norvaline, nv-5138, omithine, penicillamine, pipecolic acid, plakohypaphorine, pyroglutamic acid, quisqualic acid, s-aminoethyl-l-cysteine, sarcosine, theanine, tranexamic acid, tricholomic acid, β-alanine (3-aminopropanoic acid), or β-leucine, selenomethionine, α-amino-n-heptanoic acid, α,β-diaminopropionic acid, α,γ-diaminobutyric acid, β-amino-n-butyric acid, β-aminoisobutyric acid, N-ethyl glycine, N-propyl glycine, N-isopropyl glycine, N-methyl alanine, N-ethyl alanine, N-methyl β-alanine, N-ethyl β-alanine, α-hydroxy-γ-aminobutyric acid, trans-4-aminomethylcyclohexane carboxylic acid, and 4-hydrazinobenzoic acid. A non-proteinogenic amino acid may comprise a ring structure. A non-proteinogenic amino acid may be aliphatic, branched, or cyclic. A non-proteinogenic amino acid may be non-cyclic. A non-proteinogenic amino acid may be positively charged, for example, carry at least 1, 2, 3, 4, 5, or more positive charges. A non-proteinogenic amino acid may be negatively charged, for example, carry at least 1, 2, 3, 4, 5, or more negative charges. A non-proteinogenic amino acid may also be neutral or not carry a charge. A non-proteinogenic amino acid may comprise a side-chain chemical moiety, for example, at least 1, 2, 3, 4, 5, or more side chain chemical moieties. A linker may comprise a proteinogenic amino acid. A linker may comprise a non-proteinogenic amino acid. A linker may comprise at least and/or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80 or more proteinogenic amino acids. A linker may comprise at least and/or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80 or more non-proteinogenic amino acids. Where a linker comprises multiple amino acids, such as multiple non-proteinogenic amino acids, an amine moiety adjacent to a ring moiety (e.g., the amine moiety in the hydrazine moiety) can function as a water-solubilizing group. Other moieties can be used to increase water-solubility, such as by linking amino acids with oxamate moieties.

A linker may comprise a quaternary amine. For example, a linker may comprise at least and/or at most about 1, 2, 3, 4, 5, 6, 7, 8, 9, 10 or more quaternary amine subunits. Where multiple quaternary amine subunits are present, in some cases, they may be linked consecutively, or one or more quaternary amine subunits may be separated by other linker subunits (e.g., amino acid subunits, e.g., Hypn).

A linker may comprise a semi-rigid portion. The semi-rigid portion of the linker may provide physical separation between the substrate and the optical moiety, which physical separation may facilitate, e.g., effective labeling of the substrate with the labeling reagent, effective detection of the labeling reagent coupled to the substrate, effective labeling of the substrate with additional labeling reagents (e.g., in the case of incorporation into homopolymeric regions of a nucleic acid template), etc. For example, the semi-rigid portion may provide physical separation of, on average, at least 9 Å, 12 Å, 15 Å, 18 Å, 21 Å, 24 Å, 27 Å, 30 Å, 33 Å, 36 Å, 39 Å, 42 Å, 45 Å, 48 Å, 51 Å, 54 Å, 57 Å, 60 Å, 63 Å, 66 Å, 69 Å, 72 Å, 75 Å, 78 Å, 81 Å, 84 Å, 87 Å, 90 Å, or more between the substrate and the optical moiety. This average separation may vary with environmental conditions including, for example, solvents (or lack thereof), temperature, pH, pressure, etc. A semi-rigid portion of a linker may comprise a secondary structure such as a helical structure that establishes and maintains a degree of physical separation between the substrate and the optical moiety. The helical structure can comprise prolines and/or hydroxyprolines (e.g., polyproline or polyhydroxyproline helix). The semi-rigid portion may comprise an amino acid, e.g., non-proteinogenic amino acid. Non-proteinogenic amino acids of a linker may be included in any useful portion of the linker and may be included in sequence or separated by one or more other chemical moieties. A semi-rigid portion of a linker may comprise a series of ring systems (e.g., aliphatic and aromatic rings). As used herein, a ring (e.g., ring structure) is a cyclic moiety comprising any number of atoms connected in a closed, essentially circular fashion, as used in the field of organic chemistry. A linker, or a semi-rigid portion thereof, can have any number of rings, including at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80 or more rings. The rings can share an edge in some cases (e.g., be components of a bicyclic ring system). In general, the ring portion of the linker can provide a degree of physical rigidity to the linker and/or facilitate physical separation between objects attached to the linker. A ring can be a component of an amino acid (e.g., a non-proteinogenic amino acid, as described herein). For example, a linker may comprise a proline moiety or a hydroxyproline moiety. For example, a linker, or a semi-rigid portion thereof, may comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80 or more proline or hydroxyproline moieties. In some cases, different portions of the linkers may be separated by one or more moieties such as glycine moieties, e.g., a first hydroxyproline section of the linker may be separated from a second hydroxyproline section of the linker with a glycine moiety. A linker may comprise one or more water-soluble groups. A linker may include one or more asymmetric (e.g., chiral) centers (e.g., as described herein). All stereochemical isomers of linkers are contemplated, including racemates and enantiomerically pure linkers. A labeling reagent or component thereof, and/or an object, may include one or more isotopic (e.g., radio) labels (e.g., as described herein). All isotopic variations of linkers are contemplated.

A labeling reagent or linker can establish any suitable functional distance between an optical moiety and an object, such as at least and/or at most about 500 nm, about 200 nm, about 100 nm, about 75 nm, about 50 nm, about 40 nm, about 30 nm, about 20 nm, about 10 nm, about 5 nm, about 2 nm, about 1.0 nm, about 0.5 nm, about 0.3 nm, or about 0.2 nm. In some instances, the functional length is at least and/or at most about 9 Å, 12 Å, 15 Å, 18 Å, 21 Å, 24 Å, 27 Å, 30 Å, 33 Å, 36 Å, 39 Å, 42 Å, 45 Å, 48 Å, 51 Å, 54 Å, 57 Å, 60 Å, 63 Å, 66 Å, 69 Å, 72 Å, 75 Å, 78 Å, 81 Å, 84 Å, 87 Å, 90 Å, or more.

A linker may comprise a polymer having a regularly repeating unit. Alternatively, a labeling reagent may comprise a co-polymer without a regularly repeating unit. A repeating unit may comprise a sequence of amino acids (e.g., non-proteinogenic amino acids). A repeating unit may comprise two or more different amino acids. For example, a linker may comprise a moiety having the formula (XₙYₘ)ᵢ, where X is a first amino acid, Y is a second amino acid, n is at least 1, m is at least 1, and i is at least 2, and X and Y are different amino acids. In an example, X may be glycine, n is 1, and Y is hydroxyproline. In such an instance, m may be at least 3 (e.g., 3, 4, 5, 6, 7, 8, 9, 10, 11, 12, 13, 14, 15, 16, 17, 18, 19, 20, or more) and i may be, for example, at least 2 (e.g., 2, 3, 4, 5, 6, 7, 8, or more).

An example linker component is shown below:

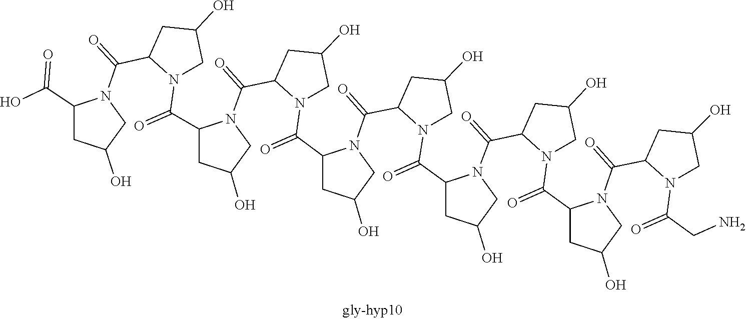 [SMILES: NCC(=O)N1CC(O)CC1C(=O)N1CC(O)CC1C(=O)N1CC(O)CC1C(=O)N1CC(O)CC1C(=O)N1CC(O)CC1C(=O)N1CC(O)CC1C(=O)N1CC(O)CC1C(=O)N1CC(O)CC1C(=O)N1CC(O)CC1C(=O)N1CC(O)CC1C(=O)O]

The following labels of: “Hypₙ”, “Hypn”, “hypₙ”, “hypn”, as used herein, which may generally describe a unit of n hydroxyproline moieties, unless explicitly described otherwise (e.g., “gly-”, “Gly-”, “Gly”-, “gly”-, “with glycine”, “without glycine”, as drawn, etc.) may refer to a structure which may or may not have one or more glycine moieties. For example, such labels may describe a structure of n hydroxyproline moieties with a glycine moiety at an end, a structure of n hydroxyproline moieties which may have one or more glycine moieties between hydroxyprolines, or a structure of n hydroxyproline moieties without any glycine moieties. The structure shown above includes 10 hydroxyproline moieties and a glycine moiety and is referred to herein as “H” “gly-hyp10”, GlyHyp10, Gly-Hyp10, glyhyp₁₀, gly-hyp₁₀, hyp10-gly, or similar. One or more such structures may be included in a labeling reagent or linker portion thereof. For example, a gly-hyp10 structure may be a repeating unit in a linker. Two gly-hyp10 structures in sequence may be referred to herein as hyp20 (having two glycines), or gly-hyp10-gly-hyp10. Such a structure may include 20 hydroxyproline moieties and, in some cases, one or more (e.g., two) glycines. Similarly, three gly-hyp10 structures in sequence may be referred to herein as gly-hyp30. Such a structure may include 30 hydroxyproline moieties and one or more glycines. For example, a gly-hyp30 sequence may include three sets of ten hydroxyprolines separated by glycines. Alternatively, a hyp30 structure may include thirty hydroxyprolines with no intervening structures. Related structures including different numbers of hydroxyprolines (e.g., hypn or hypₙ) may also be included in a labeling reagent. As described herein, all stereoisomers of gly-hyp10, gly-hyp20, and hyp30, as well as combinations thereof, are contemplated.

### Cleavable Moieties

A labeling reagent may include one or more cleavable moieties. A cleavable moiety may comprise a cleavable group such as a disulfide moiety. A cleavable moiety may comprise a chemical handle for attachment to an object (e.g., as described herein). Accordingly, a cleavable moiety may be included in a labeling reagent at a position adjacent to an object to which the labeling reagent is attached. A cleavable moiety may be coupled to a linker component of a labeling reagent via, for example, reaction between a free carboxyl moiety of the linker component and an amino moiety of a cleavable moiety (e.g., cleavable linker portion). A cleavable linker portion may be attached to an object upon reaction between a carboxyl moiety of the cleavable linker moiety and an amine moiety attached to an object to provide the substrate attached to the cleavable linker portion via an amide moiety.

A cleavable moiety may be cleaved via exposure to one or more stimuli, such as chemical (e.g., reducing agent), heat, enzymatic, light, etc. In some cases, the reducing reagent comprises tetrahydropyran, β-mercaptoethanol (β-ME), dithiothreitol (DTT), tris(2-carboxyethyl)phosphine (TCEP), Ellman's reagent, hydroxylamine, or cyanoborohydride.

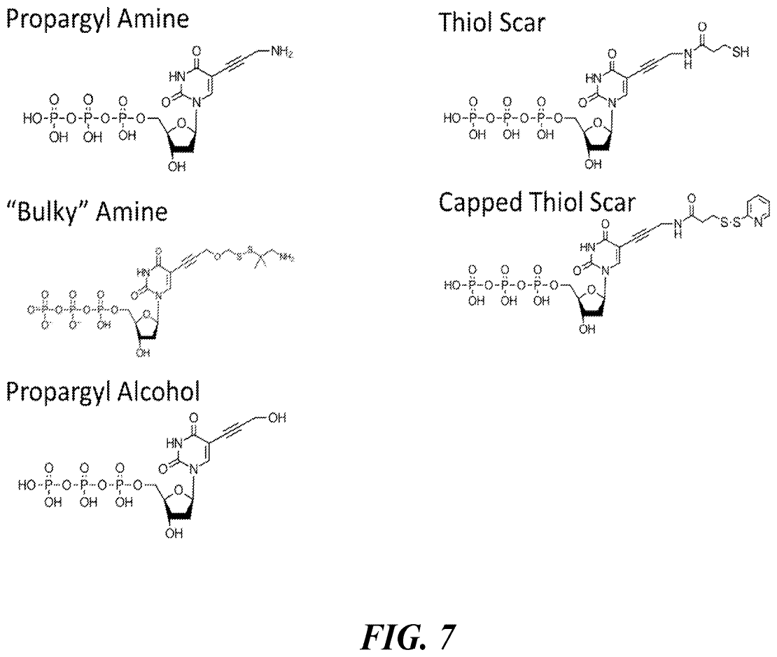

FIG. 6 illustrates different examples of cleavable groups that can be a part of a linker, Q, E, B, Y, P, M, F, W, and W′. A linker may comprise any of these cleavable group examples.

### Methods of Labeling

A reagent or object may be labeled with an optical moiety, such as a dye moiety. The optical moiety may be attached to the reagent via a linker. Thus, a labeled reagent may comprise a linker and an optical moiety. In some cases, a reagent may be labeled with a labelling reagent comprising a linker and an optical moiety. In some cases, a labeled reagent may be or comprise the labelling reagent. Labeled reagents may be detected, such as in an imaging operation. The imaging operation may comprise exciting the optical moiety (e.g., dye) using light provided at a first wavelength(s) and detecting light at a second wavelength(s).

A labeled reagent may be used to optically probe an analyte, e.g., by providing the labeled reagent to couple to or react with the analyte and detecting one or more signals deriving from the labeled reagent or reaction thereof. Coupling may be covalent or non-covalent (e.g., via ionic interactions, Van der Waals forces, etc.). In some cases, coupling may be via a linker, which may be cleavable, such as photo-cleavable (e.g., cleavable under ultra-violet light), chemically cleavable (e.g., via a reducing agent, such as dithiothreitol (DTT), tris(2-carboxyethyl)phosphine (TCEP), tris(hydroxypropyl)phosphine (THP)), or enzymatically cleavable (e.g., via an esterase, lipase, peptidase or protease). The probe may detect the presence or absence of the analyte. The probe may detect the presence or absence of a characteristic or parameter of the analyte that relates to the probe. In an example, the reagent is a nucleotide, and labeled nucleotides are used to probe a template nucleic acid to sequence the template nucleic acid (e.g., via single molecule sequencing, sequencing by synthesis, sequencing by ligation, sequencing by binding, etc.). In another example, the reagent is an oligonucleotide, and labeled oligonucleotides are used to probe a sample in order to determine the presence or absence of a gene sequence in the sample. In another example, the reagent is an antibody or oligonucleotide-conjugated antibody, and labeled antibodies or labeled oligonucleotide-conjugated antibodies are used to probe a sample to determine the presence or absence of a protein in the sample. The reagent may comprise any molecule or molecules that can be labeled by the components and mechanisms described herein. The reagent can be any suitable molecule, analyte, cell, tissue, or surface that is to be optically labeled. Non-limiting examples of reagents include cells (e.g., eukaryotic cells, prokaryotic cells, healthy cells, and diseased cells); proteins (e.g., cellular receptors; antibodies, etc.); lipids; metabolites; saccharides; polysaccharides; probes; nucleotides and nucleotide analogs (e.g., as described herein); and polynucleotides.

FIG. 6 shows a variety of components that may be used in the construction of labelling reagents and labeled reagents. A linker between the reagent and the optical moiety may comprise one or more of a cleavable linker moiety, a semi-rigid linker moiety, an amino acid, multiples thereof, or any combination thereof. FIG. 6 illustrates example nucleotide reagents, propargylamino functionalized nucleotides (A, C, G, T, and U), but any other useful nucleotide or nucleotide analog with any other useful chemical handle can be used. Non-nucleotide reagents may be labeled using the component(s) shown in FIG. 6. Cleavable linker moieties include, e.g., the structures shown as Q, E, B, Y, P, M, F, W and W′. A cleavable linker moiety may include a cleavable group (e.g., disulfide bonds) as described herein. A semi-rigid linker moiety may comprise one or more amino acid moieties, including, for example, one or more hydroxyproline moieties as described herein. A linker may comprise a hydroxyproline linker (Hypₙ). The “H” linker moiety in FIG. 6 is a hyp10 moiety. The hydroxyproline linker (Hypₙ) may comprise any useful number of hydroxyproline residues (e.g., Hyp3, Hyp6, Hyp9, Hyp10, Hyp20, Hyp30, Hyp40, etc.) and, in some cases, another moiety such as a glycine moiety, as described herein. For example, a group of consecutive hydroxyproline residues may be separated by one or more other moieties or features (e.g., [Hyp10]-[another moiety]-[Hyp10]). The amino acid moiety may comprise cysteic acid (e.g., the “Cy” moiety), 5-amino-5-carboxy-N,N,N-trimethylpentan-1-aminium or a salt thereof (e.g., the “L” moiety), 6-aminohexanoic acid (e.g., the “Am” moiety), “C” moiety, a quaternary amine (e.g., the “V” moiety or “Z” moiety), multiples thereof, or any combination thereof. A linker may include multiple portions including multiple different amino acids in any order. An optical moiety may be a fluorescent dye moiety such as the structures of “Kam”, “*,” “#,” “$,” “\hat{ }\hat{ },” or any other useful structure, such as any of the dyes or labels described elsewhere herein. Throughout the application, wherever such labels are used, any other optical moiety may be substituted. A dye may be represented as “‡,” which symbol is intended to represent any useful dye moiety or combination of dye moieties (e.g., dye pairs). A dye may be red-fluorescing or green-fluorescing.

A labeled reagent may comprise any number of linkers and any number of optical moieties. A linker may each be attached to one optical moiety (e.g., dye moiety) or multiple optical moieties (e.g., dye moieties). Multiple optical moieties on a same linker or labeled reagent may be detectable at a single wavelength or wavelength range. Multiple optical moieties on a same linker or labeled reagent may be detected at different wavelength or wavelength range. A labeled reagent may comprise a branched or dendritic structure (e.g., as described herein) comprising multiple linker moieties (e.g., multiple sets of hydroxyproline moieties connected at different branch points to a central structure), which linker moieties may be the same or different. A labeled reagent may comprise multiple dyes attached to different locations of a linker (e.g., different locations throughout a hydroxyproline moiety). A labeled reagent may comprise multiple optical moieties wherein at least one is a quencher. A linker may comprise any combination of ‘cleavable linker portion’ and ‘amino acid linker portion’ components illustrated in FIG. 6, including multiples thereof in any order. A labeled reagent may comprise any combination of ‘cleavable linker portion’ and ‘amino acid linker portion’ components illustrated in FIG. 6, including multiples thereof in any order. There are numerous possible variations of linkers and labeled reagents that may be constructed using various permutations of the components illustrated in FIG. 6, appreciating that the various linker components can be ordered in any number, any order, and in combination with or without additional moieties (e.g., such as a glycine moiety) disposed at various locations. Labeled reagents may be prepared according to synthetic routes and principles described herein. Provided herein are also unlabeled reagents. Provided herein are also mixtures of labeled and unlabeled reagents (e.g., a mixture of labeled and unlabeled nucleotides). In some cases, the reagent is a nucleotide. Any natural nucleotide, modified nucleotide, or nucleotide analog may be the reagent, such as a reversibly terminated nucleotide or unterminated nucleotide. Various linkers, labeling reagents, labels, reagents, and combinations thereof are described in further detail in U.S. Pat. No. 11,377,680, U.S. application Ser. No. 18/111,220, and International Patent Application No. PCT/US2023/013634, each of which is incorporated by reference herein in its entirety.

### Chemical Scars

The cleavage of a cleavable group may leave a scar group associated with substrate. The cleavable group can be, for example, an azidomethyl group capable of being cleaved by an agent such as tris(2-carboxyethyl)phosphine (TCEP), dithiothreitol (DTT), or tetrahydropyranyl (THP) to leave a hydroxyl scar group. The cleavable group can be, for example, a disulfide bond capable of being cleaved by an agent such as TCEP, DTT or THP to leave a thiol scar group. The cleavable group can be, for example, a hydrocarbyldithiomethyl group capable of being cleaved by an agent such as TCEP, DTT or THP to leave a hydroxyl scar group. The cleavable group may comprise a photocleavable moiety. For example, the cleavable group can be, for example, a 2-nitrobenzyloxy group capable of being cleaved by ultraviolet (UV) light to leave a hydroxyl scar group. A linker or a labeled reagent comprising the linker may be stable in the absence of an agent, light (e.g., ultraviolet light), or condition (e.g., a particular pH range) capable of cleaving a cleavable linker. For example, a linker comprising a cleavable disulfide group may be stable in the absence of a reducing agent.

A residual portion of a linker remaining on the substrate following cleavage of the cleavable group may be referred to as a ‘scar’ or as a cleaved linker. In an example, prior to labeling, an object may be functionalized to include a functional handle that is subsequently used to couple the substrate to a linker. Following cleavage and a post-cleavage reaction (e.g., an immolation reaction), such a functional handle may be part of a scar or a cleaved linker. A scar of a biomolecule (e.g., nucleotide) may comprise a portion of the biomolecule not typically associated with a canonical biomolecule of the same type (e.g., A, T, G, C, U nucleotide).

In some cases, a scar may alter a property of an object. For example, a scarred (i.e., scar-containing) nucleotide within a nucleic acid may inhibit further nucleotide incorporations into the nucleic acid. The scarred nucleotide may inhibit nucleotide incorporations at an immediately adjacent open position or may inhibit multiple subsequent nucleotide additions. In some cases, a scar may affect an optical property of an object. For example, a scar may quench fluorescence activity. In some cases, a scar may be reactive toward another species in a system, which may alter the performance of a system. For example, a nucleotide-bound scar may comprise a reactivity toward lysines, and thereby inhibit polymerase activity in a system. Thus, in some cases, performance of downstream operations (e.g., sequencing) can be enhanced by optimizing a scar's structure and properties. Chemical scars and various methods for addressing them are described in further detail in International Patent Pub. WO2022/212408A1, which is entirely incorporated herein by reference.

A scar may be stable upon cleavage. A scar may also be reactive. The scar's reactivity may be an intramolecular reactivity. In such cases, a scar may undergo a post-cleavage reaction to form a structure distinct from the initial scar formed upon cleavage. Such a post-cleavage reaction may be referred to as “immolation,” and scars which have undergone immolation may be referred to as “immolated scars.” In some cases, a scar may disappear altogether post-immolation. A linker may spontaneously immolate (i.e., undergo immolation) upon cleavage, or may form a first scar that is stable until it is contacted with a reagent or a specific condition (e.g., a specific pH range). Immolation may change a physical or chemical property of the scar group, and further may diminish its size. An immolated scar may comprise different properties than the post-cleavage scar from which it formed, which may make the immolated scar more favorable for a particular assay. In some cases, an immolated scar may inhibit an enzymatic activity (e.g., polymerase activity) less than the post-cleavage scar from which it formed. For example, using BST type polymerases for incorporations, thiol and propargyl alcohol scars (which can form directly from linker cleavage) can inhibit polymerization more than propargyl amine and primary aliphatic amine scars (which may be formed through scar immolation). In some cases, a less acidic scar (e.g., a scar comprising a higher pH) may inhibit an enzymatic activity less than a more acidic scar. In some cases, a smaller (e.g., lower mass, volume, or length) scar may inhibit an enzymatic activity less than a more acidic scar.

A strategy for mitigating an adverse effect of a scar is scar immolation. A scar may be configured to undergo a reaction subsequent to cleavage (e.g., an immolation reaction), which may alter a chemical or physical property of the scar. The immolation reaction may be initiated or accelerated by a reagent (e.g., a catalyst or reagent), light, or a condition (e.g., a pH range). The immolation reaction may be spontaneous. The immolation reaction may diminish the size of the scar. For example, an immolation reaction of a thiol-containing scar may result in the loss of the thiol moiety as a thiirane or thietane. As such, an immolation reaction may diminish the steric bulk of a scar. An immolation reaction may alter a chemical or physical property of a scar. For example, a thiol-containing scar may form a more polar and less acidic propargyl amine scar upon immolation. In some cases, a scar may be a thiol scar. In some cases, a scar may undergo an immolation scar to yield an immolated scar which comprises a primary amine or a primary hydroxyl moiety (e.g., comprising propargyl alcohol).

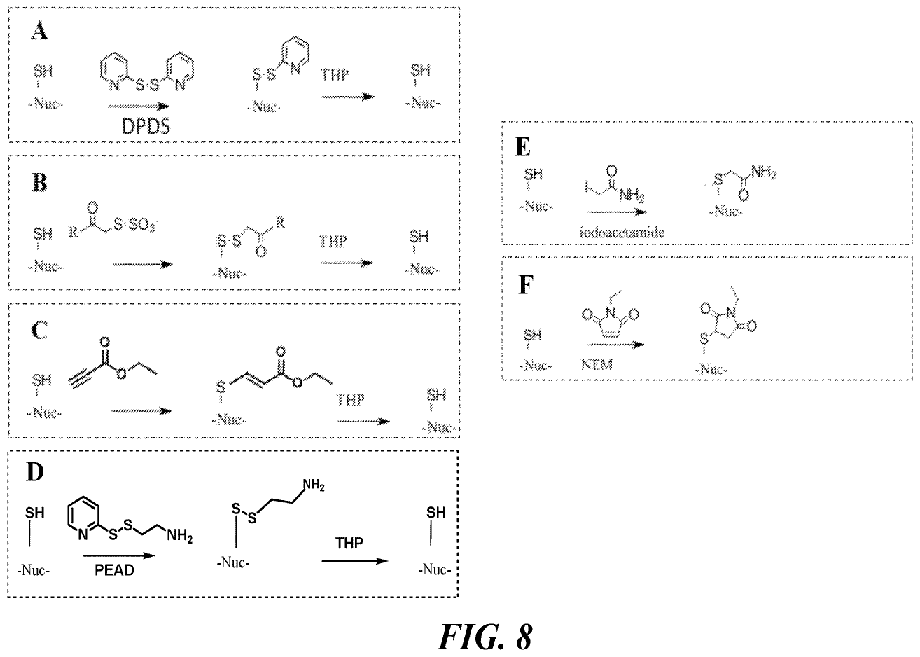

An alternative or additional strategy for mitigating an adverse (e.g., an inhibitory or mispair-inducing) effect of a scar is scar-capping. A physical or chemical property of a scar may be altered by coupling the scar to a capping reagent. The altered property may be favorable (e.g., relative to the uncapped, scarred substrate) for nucleic acid polymerization. For example, the altered physical or chemical property may diminish the inhibitory effect of a scar. The altered physical or chemical property may diminish the rate of nucleotide misincorporation into a growing nucleic acid molecule comprising the capped scar. Accordingly, a sequencing method may comprise contacting a nucleic acid molecule complex (e.g., sequencing primer-template nucleic acid complex which has incorporated a labeled reagent) with a capping reagent. A capping reagent may be selective for a scar, and therefore may be added with a labeled nucleotide substrate, with a cleavage reagent, or subsequent to a cleavage reagent. A nucleic acid polymerization method may comprise a capping reagent addition prior to or following a labeled nucleotide incorporation. In some cases, a scar comprises a thiol scar. The capping reagent may comprise a disulfide configured to react with the thiol scar. FIG. 7 illustrates different examples of scarred nucleotides, with a propargyl amine scar, a bulky amine scar, a propargyl alcohol scar, a thiol scar, and a capped thiol scar.

The capping reagent may be added with a labeled nucleotide, unlabeled nucleotide, with a cleavage reagent, subsequent to a cleavage reagent, or subsequent to a reagent, light-input, energy-input, or change in condition for a scar immolation reaction. The capping reagent may be added subsequent to a labeled nucleotide. The capping reagent may be added with an unlabeled nucleotide. For example, a method may comprise first contacting a nucleic acid with a labeled nucleotide, and then subsequently contacting the nucleic acid with a capping reagent and an unlabeled nucleotide of the same canonical type as the labeled nucleotide. Such a method may increase the likelihood of complete extension across homopolymeric regions of a template nucleic acid. The capping reagent may remain stably bound to the scarred nucleotide through subsequent nucleotide additions and cleavage steps.

The capping reagent may covalently (e.g., form a bond with) or non-covalently couple to the scar group. A capping reagent may covalently couple to a nucleophilic moiety on a scar, such as a hydroxyl or thiol. A capping reagent may reversibly or irreversibly couple to a scar. Examples of reversibly-binding capping reagents (“reversible capping reagents) include

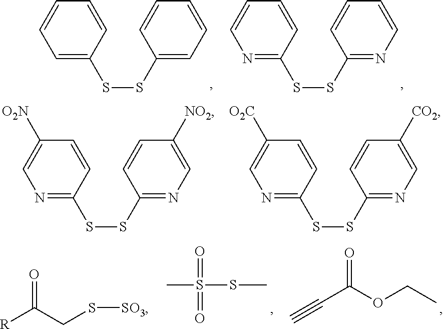

and optionally substituted (e.g., alkylated, halogenated, or carboxylated) variants thereof. It will be appreciated that examples of capping reagents include various isomers of the above, such as the 2-isomers and 4-isomers (e.g., pyridyldithio isomers), and their optionally substituted variants. Some examples of 4-isomers include:

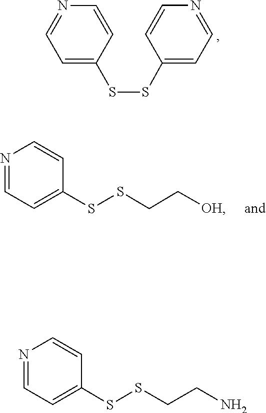 [SMILES: c1cc(SSc2ccncc2)ccn1 ; OCCSSc1ccncc1 ; "and" ; NCCSSc1ccncc1]

or (4-(4-pyridyldithio)pyridine), (2-(4-pyridyldithio)ethanol), and 2-(4-pyridyldithio)ethylamine.

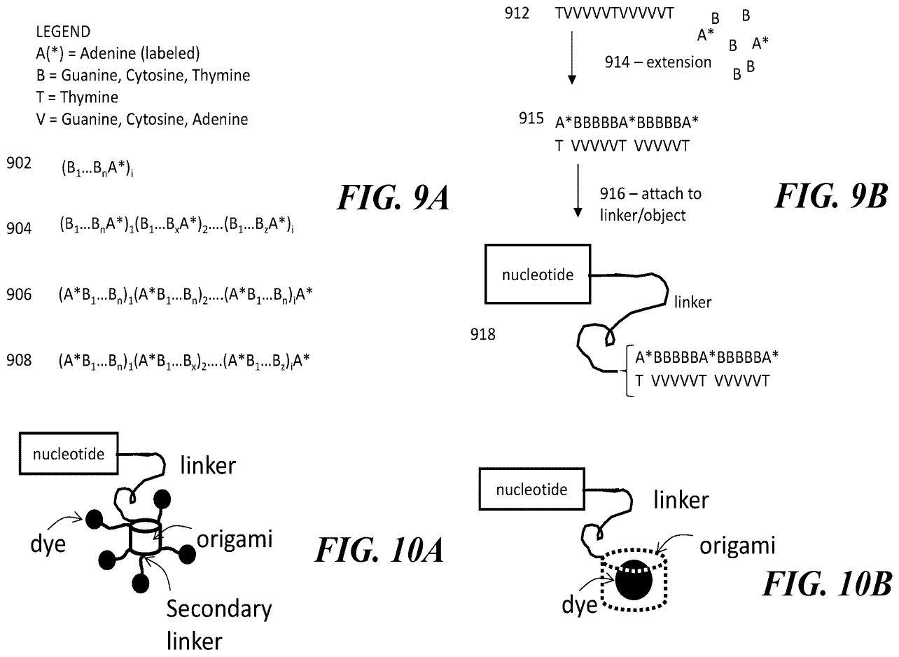

A reversible thiol capping reagent may comprise a disulfide, a thiosulfate, or an alkyne, and may cap a thiol scar through for example a thiol-disulfide exchange, as illustrated in FIG. 8 Panels A, B, D, or a thiol-yne reaction, as illustrated in FIG. 8 Panel C. Reversible capping of a thiol scar may convert the thiol into a disulfide. The disulfide may subsequently be cleaved by a reducing agent, such as THP. In some cases, a single reagent may cleave a cleavable linker and remove a reversible capping reagent. For example, a reducing reagent such as THP may remove a thiolate (e.g., a pyridine thiolate derived from a dipyridyldisulfide capping reagent or a benzenethiolate derived from a diphenyldisulfide capping reagent). A capping reagent or a portion thereof (e.g., a methyl or acetyl group) may irreversibly couple to a scar. In some cases, irreversible coupling denotes formation of a stable bond in the conditions of and upon contact with the reagents for a particular assay. For example, a hydroxyl scar methylating reagent may be an irreversible capping reagent in a nucleic acid polymerization assay if none of the conditions or reagents of the assay are configured to remove a methyl group from a methoxide moiety. An irreversible thiol capping reagent may comprise an iodoacetyl or pyrrole dione moiety. Examples of irreversible thiol capping reagents include

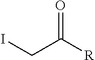

(wherein R may comprise O, S, optionally substituted alkyl, optionally substituted alkenyl, optionally substituted alkynyl, optionally substituted amine, optionally substituted alkoxide, cycloalkyl, optionally substituted heterocycloalkyl, optionally substituted aryl, and optionally substituted heteroaryl),

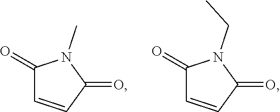

and optionally substituted (e.g., alkylated, halogenated, or carboxylated) variants thereof. An irreversible thiol capping reagent may comprise a substitutable halogen (e.g., iodide in iodoacetamide) or an electrophilic olefin (e.g., the double bonded carbons of a pyrrole dione), and may form a carbon-sulfur bond between the thiol scar and the capping reagent or a portion thereof. Exemplary capping reagents include, but are not limited to, ethyl propiolate (EP), iodoacetamide (IAc), methyl methanethiosulfonate (MMTS), dipyridyl disulfide (DPDS), 4-4′-dipyridyl disulfide, 2,2′-dithiobis(5-nitropyridine), 6,6′-dithiodinicotinic acid, and pyridyl ethyl amine disulfide (PEAD).

### Sequencing Using Labeled Reagents

The labeled objects of the present disclosure may be used to sequence a template nucleic acid. For example, the labeled objects may comprise labeled nucleotides. The template nucleic acid may be sequenced while attached to a support (e.g., bead). Alternatively, the template nucleic acid may be free of the support when sequenced and/or analyzed. The template nucleic acid may be sequenced while immobilized to a substrate (e.g., a wafer), such as via a support or otherwise. Any sequencing method may be used, for example pyrosequencing, single molecule sequencing, sequencing by synthesis (SBS), sequencing by ligation, sequencing by binding, etc.

Sequencing may comprise extending a sequencing primer (or growing strand) hybridized to a template nucleic acid by providing labeled nucleotide reagents, washing away unincorporated nucleotides from the reaction space, and detecting one or more signals from the labeled nucleotide reagents which are indicative of an incorporation event or lack thereof. After detection, the labels may be cleaved and the whole process may be repeated any number of times to determine sequence information of the template nucleic acid. One or more intermediary flows may be provided intra- or inter-repeat, such as washing flows, label cleaving flows, terminator cleaving flows, reaction-completing flows (e.g., double tap flow, triple tap flow, etc.), labeled flows (or bright flows), unlabeled flows (or dark flows), phasing flows, chemical scar capping flows, etc. A nucleotide mixture that is provided during any one flow may comprise only labeled nucleotides, only unlabeled nucleotides, or a mixture of labeled and unlabeled nucleotides. The mixture of labeled and unlabeled nucleotides may be of any fraction of labeled nucleotides, such as at least or at most 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 99%. A nucleotide mixture that is provided during any one flow may comprise only non-terminated nucleotides, only terminated nucleotides, or a mixture of terminated and non-terminated nucleotides. When using only non-terminated nucleotides, terminator cleaving flows may be omitted from the sequencing process. When using terminated nucleotides, to proceed with the next step of extension, prior to, during, or subsequent to detection, a terminator cleaving flow may be provided to cleave blocking moieties. A nucleotide mixture that is provided during any one flow may comprise any number of canonical base types (e.g., A, T, G, C, U), such as a single canonical base type, two canonical base types, three canonical base types, four canonical base types or five canonical base types (including T and U). Different types of nucleotide bases may be flowed in any order and/or in any mixture of base types that is useful for sequencing. Various flow-based sequencing systems and methods are described in U.S. Pat. No. 11,459,609, which is incorporated by reference herein in its entirety. Nucleotides of different canonical base types may be labeled and detectable at a single frequency (e.g., using the same or different dyes). In other cases, nucleotides of different canonical base types may be labeled and detectable at different frequencies (e.g., using the same or different dyes).

Subsequent to sequencing, the sequencing signals collected and/or generated may be subjected to data analysis. The sequencing signals may be processed to generate base calls and/or sequencing reads. The sequencing reads may be processed to generate diagnostics data to the biological sample, or the subject from which the biological sample was derived from. The data analysis may comprise image processing, alignment to a genome or reference genome, training and/or trained algorithms, error correction, and the like.

### Nucleic Acid-Based Labeled Reagents

A reagent or an object may be indirectly coupled to one or more detectable moieties. The reagent may be a nucleic acid base for use in sequencing. The one or more detectable moieties may be used to detect incorporation or lack thereof of a reagent into an extending sequencing primer (e.g., by a polymerase). By increasing the distance between the reagent and the label (e.g., by using longer linkers, indirect linking, linkers with rigid structures, etc.), it is possible to increase the efficiency of incorporation (e.g., by reducing size and/or structure-based occlusion of a polymerase's active site). It may be desirable to couple multiple detectable moieties to a single reagent (e.g., to increase the brightness). However, the presence of multiple fluorophores can lead to quenching (e.g., proximity quenching may result in reduction in detectable signal based on electric interactions between the fluorophores, where the signal reduction is determined from the expected detectable signal from the multiple fluorophores). One particular issue with quenching is that it causes uncertainty in the meaning of detected signal (e.g., how many reagents were incorporated at a single timepoint). Various methods and compositions for reducing quenching and/or increasing brightness of labels attached to substrates are described in International Patent Application No. PCT/US2023/013634, which is incorporated by reference herein in its entirety. However, there remains a need for labeled reagents that exhibit increased brightness, decreased quenching, and other desirable physical and chemical characteristics.

Provided herein are systems, methods, compositions, and kits comprising nucleic acid-based, labeled reagents (e.g., reagents comprising one or more nucleic acid moieties indirectly or directly linking the reagent to one or more detectable moieties). Nucleic acid moieties may be one or more DNA origami constructs, one or more oligonucleotides, or a combination thereof. Labels (e.g., detectable moieties) may be coupled to the substrates via linkers, as described elsewhere herein, and via the nucleic acid moieties. Such systems, methods, compositions, and kits can be useful for sequencing.

### Oligo-Based Labeling

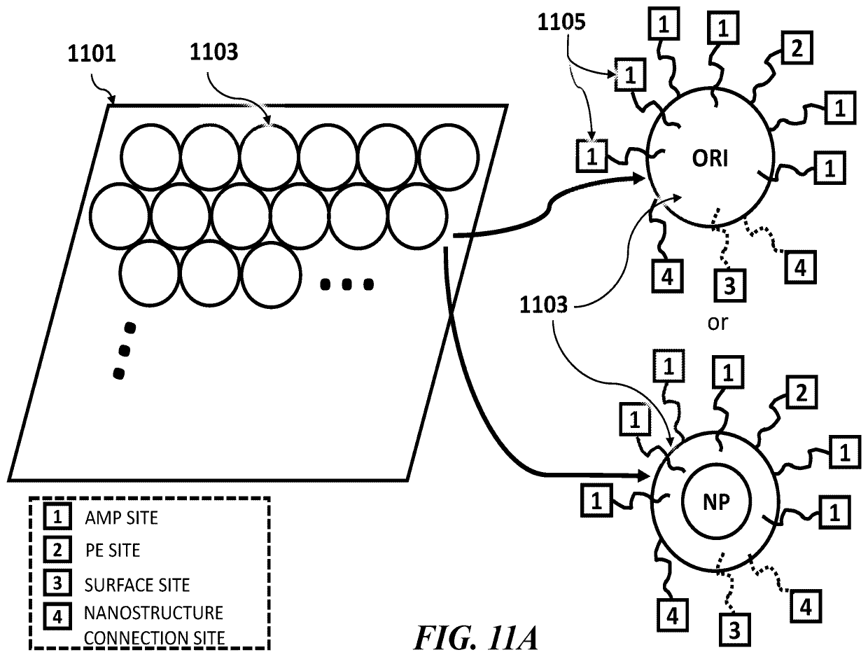

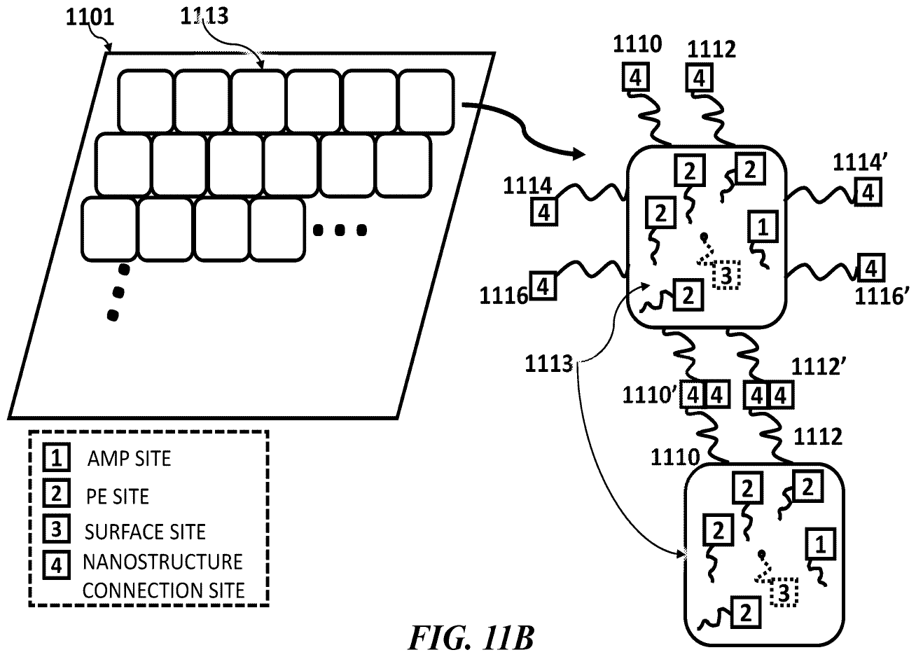

FIGS. 9A and 9B illustrate examples of labeled reagents comprising an oligonucleotide (e.g., where the nucleic acid moiety is an oligonucleotide). Dye-labeled nucleotides (e.g., of a first canonical base type) that may be used in accordance with the methods described in FIGS. 9A and 9B are detailed in Example 3.

In FIG. 9A, various organizations for a single strand of a labeled oligonucleotide are provided. In these cases, a first canonical base type, here adenine (A*) is labeled with a detectable moiety. In these examples, these labeled strands comprise one or more labeled adenines interspersed with unlabeled nucleotides that are not adenine (i.e., thymine, guanine, or cytosine). In 902, the unit of organization is one or more of a second canonical base type or types (e.g., represented by B, which is not adenine) and a labeled adenine (A*). The one or more of the second canonical base type may be any number n. In some cases, n is an integer between 1 and 20. In some cases, n may be an integer greater than 20. Unit 902 may be repeated any number of times i. In some cases, i may be an integer between 1 and 10. Each repetition of unit 902 increases by 1 the number of detectable moieties present in the single strand of a labeled oligonucleotide. By way of example, in a case where n is 4 and i is 3 the sequence would be:

(SEQ ID NO: 4)
BBBBA*BBBBA*BBBBA*.

In 904, the number n of unlabeled nucleotide bases may vary in one or more of the i repetitions. For instance, one possible sequence is BBBA*BBA*BBBA*BA* (SEQ ID NO:6). It will be appreciated that there are many possible sequences, e.g., where n varies in each repetition, where n varies in at least one repetition, where n varies according to a pattern (e.g., n=(2, 3, 2, 3)).

Example oligonucleotides 906 and 908 differ in that the labeled nucleotide begins the sequence. It will be appreciated that different organizations than those listed here are possible.

In all of these examples, the nucleotide that is coupled to one or more detectable moieties may be of any canonical base type. That is, in some cases the labeled nucleotide(s) may be adenine, thymine, guanine, cytosine, or any nucleotide analog. In some cases, the labeled nucleotides may be of two canonical base types (e.g., A and T). In some cases, the labeled nucleotides may be of three canonical base types (e.g., A, T, and C). That is, in some such cases, there may be bases of two or three canonical base types that are labeled.

Similarly, the unlabeled nucleotides may all be of a single canonical base type, of two canonical base types, or of three canonical base types; however, the unlabeled nucleotides will not be of the same canonical base type(s) as the labeled nucleotides. That is, in all cases, at least one canonical base type that is present in the sequence will be unlabeled. For instance, if the first canonical base type is C, then the unlabeled bases may be any of G, T, and A.

In FIG. 9B, provides an exemplary method for producing a labeled oligonucleotide in accordance with the organizations shown in FIG. 9A. Unlabeled oligonucleotides may be ordered from a commercial source or synthesize. The strand including labeled nucleotide bases may be synthesized using the unlabeled oligonucleotide as a template. This permits strict control over the number of labeled nucleotides that are included in the labeled, double-stranded oligonucleotide. The resulting double stranded labeled molecule may be used to label a reagent. In some cases, the synthesized labeled strand of the oligonucleotide may be used in single stranded form (e.g., denatured from the unlabeled strand and purified) to label a reagent.

In FIG. 9B, an unlabeled oligonucleotide 912 is provided. Here the sequence of oligonucleotide 912 is TVVVVVTVVVVVT (SEQ ID NO:1), where T is thymine and V is not thymine and may be any combination of cytosine, adenine, and guanine. In some cases, oligonucleotide 912 may further comprise a primer binding site (e.g., for binding a primer to enable polymerase extension). Oligonucleotide 912 may be exposed to conditions for extension 914, where the extension reagents include two or more canonical base types, e.g., a first canonical base type comprising labeled nucleotide bases and a set of additional nucleotide bases including a second canonical base type. In some cases, the set of additional nucleotide bases comprises one, two, or three canonical base types different from the first canonical base type. The product oligonucleotide of extension 914 is double stranded and comprises the original strand (e.g., oligonucleotide 912) and the synthesized strand comprising one or more labeled nucleotides of the first canonical base type. The labeled strand of the extension product 915 comprises the organizational unit (B₅A*)₂ (i.e., n=5 and i=2). The double stranded, labeled oligonucleotide 915 may be further coupled 916 to a linker and/or a reagent. The linker may be any linker described herein. The reagent may be any reagent described herein. In some cases, the linker further comprises one or more cleavable moieties. The resulting labeled reagent 918 may be used in sequencing.

### DNA Origami-Based Labeling

DNA origami refers to nucleic acid nanostructures that may self-assemble into predetermined organizations and are particularly useful for organizing other nanoparticles (e.g., by their inherent structural properties). DNA origami allows for precise control of the shape of the resulting nanostructures (e.g., selecting particular sequences). Details on DNA origami can be found, e.g., in Agarwal and Gopinath. 2022. DNA origami 2.0. bioRxiv doi: 10.1101/2022.12.29.522100; Engelhardt et al. 2019. Custom-Size, Functional, and Durable DNA Origami with Design-Specific Scaffolds. ACS Nano. 13, 5015-5027; and Han et al. 2011 DNA Origami with Complex Curvatures in Three-Dimensional Space. Science 332, 342-346, each of which is incorporated by reference herein in its entirety.

DNA origami typically comprises one or more single stranded nucleic acid molecules. These single stranded molecules, due to sequence complementarity, will hybridize to each other, thus folding into the desired shape. Typically, assembly begins with a longer, single stranded nucleic acid molecule (e.g., a “scaffold”) that may be circular (e.g., a single stranded plasmid shape) or linear. One or more shorter, single stranded nucleic acid molecules (e.g., “staples”) with sequence complementarity to regions of the scaffold and/or to each other may be added. These hybridizations will result in sequence-induced conformational changes to the scaffold, thus producing the desired nucleic acid nanostructure.

FIG. 4 and FIG. 5 illustrate two examples of labeled reagents comprising DNA origami structures (i.e., where the nucleic acid moiety is a DNA nanostructure).

In FIG. 4, a reagent is coupled to a linker, the linker is coupled to a DNA nanostructure, and the DNA nanostructure is coupled to one or more detectable moieties, thus providing a labeled reagent. In this case, the DNA nanostructure 404 comprises one or more attachment sites 406 where the detectable moieties may be any label described herein. The DNA nanostructure may be any structure suitable for adhering to the one or more detectable moieties (e.g., any suitable DNA origami structure). The DNA nanostructure may be two dimensional. The DNA nanostructure may be three dimensional.

In FIG. 5, a reagent is coupled to a linker, the linker is coupled to a DNA nanostructure 504, and the DNA nanostructure encloses at least one detectable moiety 502. The DNA nanostructure may be any structure suitable for enclosing to the one or more detectable moieties (e.g., any suitable DNA origami structure). The DNA nanostructure may be two dimensional. The DNA nanostructure may be three dimensional.

### Substrate Systems and Methods

One of the key advantages of DNA origami is its ability to create a wide range of nanoscale shapes, including squares, triangles, stars, and even more complex structures like smiley faces or letters. The technology can also be used to create nanoscale three-dimensional shapes, including but not limited to pyramids, tetrahedrons, cones, etc. DNA origami technique has found applications in various fields, including nanoelectronics, drug delivery, and biophysics.

In nanoelectronics, DNA origami has been explored as a platform for the precise placement of nanoscale components, such as carbon nanotubes or nanoparticles, to create functional devices and circuits. In drug delivery, DNA origami structures can be engineered to encapsulate and deliver therapeutic agents with high precision, potentially revolutionizing targeted drug delivery systems. Moreover, in biophysics, DNA origami serves as a valuable tool for studying fundamental biological processes and interactions at the nanoscale.

The versatility, programmability, and biocompatibility of DNA origami make it a promising tool for various scientific and technological applications, pushing the boundaries of what is possible in the world of nanotechnology. As researchers continue to refine and expand upon the capabilities of DNA origami, its potential impact on fields ranging from medicine to materials science is poised to grow.

Creating DNA origami involves a series of well-established methods that can include but are not limited the following:

Designing DNA Sequences: At this step different DNA sequences are designed in order to create different types of shapes. DNA origami design often utilizes software tools like caDNAno and others for designing scaffold and staple sequences. caDNAno was first initially developed in William Shih's laboratory at the Dana Farber Cancer Institute and can be downloaded from the cadnano.org website. Additional information can be found in Rothemund, P. “Folding DNA to create nanoscale shapes and patterns,” Nature, Vol 440: 297-302 (2006), which is incorporated by reference herein in its entirety.

Assembly Planning: Computational tools aid in optimizing staple strand placement. Tools like CanDo (Computer-aided engineering for DNA origami), which is available from cando-dna-origami.org, assist in assist in optimizing staple strand placement to achieve the desired structure. Additional information can be found in Kim D N et al., “Quantitative prediction of 3D solution shape and flexibility of nucleic acid nanostructures,” Nucleic Acids Research, 40(7):2862-2868 (2012), which is incorporated by reference herein in its entirety.

Chemical Synthesis: DNA strands are chemically synthesized using techniques such as solid-phase synthesis. Commercial services or in-house synthesis methods are employed (for instance, using phosphoramidite chemistry).

Annealing and Folding: Annealing is achieved by mixing the synthesized DNA strands in buffer solutions under controlled conditions, allowing for the self-assembly of the origami structure. Additional information can be found in Douglas S M et al., “Self-assembly of DNA into nanoscale three-dimensional shapes,” Nature Vol 459: 414-418 (2009), which is incorporated by reference herein in its entirety.

Verification and Purification: Gel electrophoresis and atomic force microscopy (AFM) may be used to purify, verify, and characterize folded DNA origami structures. This may be beneficial to select for desired sizes and/or shapes of nanostructures.

Characterization and Imaging: High-resolution imaging on the scale required for DNA nanostructure resolution may comprise atomic force microscopy (AFM) or transmission electron microscopy (TEM). These techniques permit the visualization of DNA origami scaffolds.

Functionalization (Optional): Additional functionalization may involve the conjugation of other molecules or nanoparticles to specific sites on the origami. This step depends on the intended application. For example, specific probes, antibodies, complementary nucleic acid sequences can be added to an origami structure to capture certain targets.

Application-Specific Modifications (Optional): For specific applications, researchers may integrate additional components into the origami structure. In nanoelectronics, this could include incorporating nanowires or quantum dots.

Additional information can be found in, for example, Hong F. et al., “DNA origami: scaffolds for creating higher order structures,” Chem. Rev. 117: 12584-12640 (2017) and Dong Y et al., “Preparation of irregular silica nano-abrasives for the chemical mechanical polishing behaviour on sapphire substrates,” Micro & Nano Letters 14 (13): 1328-1333 (2019), each of which is incorporated by reference herein in its entirety.

### DNA Nanostructures as Supports

Provided herein are devices, systems, methods, compositions, and kits that use DNA nanostructures as supports to immobilize nucleic acids for sequencing. Such devices, systems, methods, compositions, and kits can be applied alternatively or in addition to the sequencing workflow 100 of FIG. 1. Such devices, systems, methods, compositions, and kits can be used in conjunction with the sample processing systems and methods, or components thereof (e.g., substrates, detectors, reagent dispensing, continuous scanning, etc.) described herein.

A DNA nanostructure may be a 2- or 3-dimensional structure which shape, size, and surface functionality can be programmed with high precision. For example, a molecule or a number of molecules may be precisely disposed at or attached to a pre-determined location on a surface of the DNA nanostructure. The DNA nanostructure may comprise a plurality of pre-determined locations for placement of a plurality of molecules, which molecules may be the same type of molecule or different types of molecules. In some cases, a DNA nanostructure may comprise at least and/or at most about an order of 1, 10, 10², 10³, 10⁴, 10⁵, 10⁶, 10⁷, 10⁸, 10⁹ or more predetermined locations that can function as molecule attachment sites. The pre-determined molecule may be used to attach any molecule, such as an organic, inorganic, biological, or non-biological molecule. A DNA nanostructure may also be referred to herein as a DNA nanoparticle, DNA origami structure, or DNA origami particle.

A DNA nanostructure may be constructed using DNA origami techniques. In one example, a DNA nanostructure may be assembled or self-assembled using a long single-stranded DNA oligonucleotide which acts as a scaffold or backbone strand and a plurality of short single-stranded DNA oligonucleotides that acts as staple strands. The staple strands may attach to the scaffold strand in particular structural configurations to form an organized, engineered DNA nanostructure. The staple strands may comprise the same or different oligonucleotides. A DNA nanostructure may be constructed using a single scaffold strand or a plurality of scaffold strands. In some cases, a DNA nanostructure may comprise a silica shell at least in part or in whole. A DNA nanostructure may comprise any number of pre-determined locations, such as with functional ligands, to attach molecules.

A DNA nanostructure used herein may comprise a cross-link or other linker to stabilize the nanostructure. The cross-link or other linker may or may not be reversible, such as by applying one or more stimuli, including light stimuli, heat stimuli, chemical stimuli, magnetic stimuli, electrical stimuli, and other stimuli, or combination thereof. In some cases, the DNA nanostructure may comprise a photo-cross-link. A photo-cross-link may be generated by a photo cross-linking reaction. In some instances, an oligodeoxynucleotide (ODN) comprising 3-cyanovinylcarbazole nucleoside (^{CNV}K) can be subjected to photoirradiation conditions to photo-cross-link a target pyrimidine and the ^{CNV}K. In some instances, irradiation is provided at 366 nm for about 1 second for photo-cross-linking to thymine, and for up to about 25 seconds for photo-cross-linking to cytosine. In this example, irradiation provided at 312 nm for about 3 minutes can reverse the cross-link. Various other cross-linking reagents may be used to generate a cross-link (e.g., chemical cross-link).

A DNA nanostructure used herein may be capped in one or more locations for non-extension, such as with a terminal dideoxy NTP (ddNTP). Beneficially, when a DNA nanostructure comprising a template nucleic acid is subjected to nucleic acid extension reactions, structural components of the DNA nanostructure will not also extend with the intended extending molecule on the DNA nanostructure.

Provided herein are systems and methods for pre-enrichment using DNA nanostructures. Pre-enrichment methods have been described elsewhere herein, such as with respect to operation 102 in FIG. 1, in which template nucleic acids are attached to supports to prepare for amplification. A method of pre-enrichment may comprise attaching a template nucleic acid to a support, wherein the support comprises a DNA nanostructure, the DNA nanostructure comprising (1) a pre-enrichment site configured to bind to the template nucleic acid and (2) an amplification site configured to bind an amplified derivative of the template nucleic acid, wherein there are fewer pre-enrichment site(s) than amplification site(s). A template nucleic acid may be configured to bind to the pre-enrichment site and not to the amplification site. For example, the pre-enrichment site may comprise a first oligonucleotide molecule comprising (1) a first sequence comprising a capture sequence complementary to an adapter sequence of the template nucleic acid and (2) a second sequence comprising an amplification primer sequence (e.g., forward primer, reverse primer). The amplification site may comprise a second oligonucleotide molecule comprising the second sequence comprising the amplification primer sequence but not the first sequence comprising the capture sequence complementary to the adapter sequence. As a result, the template nucleic acid may only be able to bind to the first oligonucleotide molecule at the pre-enrichment site and not to the second oligonucleotide molecule at the amplification site. Upon hybridization of the template nucleic acid, (1) the first oligonucleotide may be extended using the template nucleic acid as a template to generate a first extended molecule comprising the first sequence and a complement of the template sequence, and (2) the template nucleic acid may be extended using the first oligonucleotide molecule as a template to generate a second extended molecule comprising the template sequence and a complement of the first sequence. The second extended molecule (e.g., an amplified derivative of the template nucleic acid) may then be removed (e.g., denatured) from the first extended molecule and it or its derivatives may be able to bind to the second oligonucleotide molecule at the amplification site via hybridization of the second sequence and the complement of the second sequence.

The DNA nanostructure may comprise a plurality of amplification sites to bind a plurality of, e.g., a colony of, amplified derivatives of the template nucleic acid. Each pre-enrichment site may comprise the first oligonucleotide molecule, as described above. Each amplification site may comprise the second oligonucleotide molecule, as described above. In some cases, the DNA nanostructure may comprise only a single, a few, or significantly less pre-enrichment site(s) compared to amplification site(s). For example, there are at most 50%, 45%, 40%, 35%, 30%, 25%, 20%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.0010%, 0.00010% or less percentage of pre-enrichment sites from all attachment sites (including all pre-enrichment and amplification sites). Beneficially, when a plurality of template nucleic acids is contacted with a plurality of DNA nanostructures comprising such pre-enrichment site % composition, it is more likely that a DNA nanostructure binds to at most one template nucleic acid than binding to multiple template nucleic acids. A DNA nanostructure that binds to multiple template nucleic acids (e.g., 2, 3, 4, 5, or more) is not favored as once the complex is subjected to amplification, it will lead to multi-clonally amplified support that produces mixed signals and adds to sequencing noise. Thus, the pre-enrichment methods described herein may advantageously generate single-template attached support-template complexes.

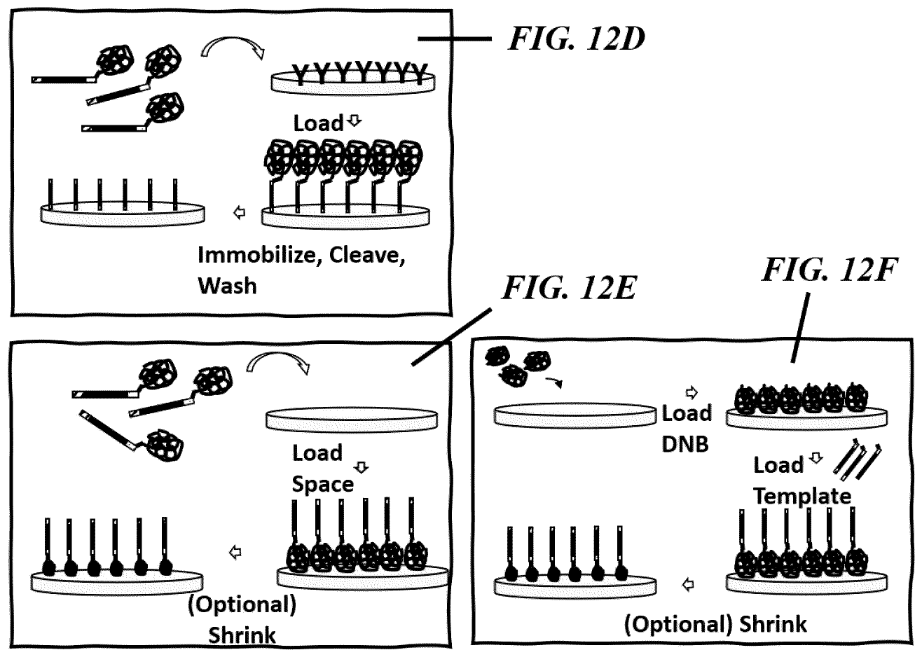

FIG. 11A illustrates example DNA nanostructures that can be used as supports. A DNA nanostructure 1103 may comprise a plurality of attachment sites 1105, for example, [1] amplification (AMP) site(s) and [2] pre-enrichment (PE) site(s). In the illustration, the DNA nanostructures comprises many [1] amplification sites and few or single [2] pre-enrichment site(s). In some cases, the plurality of attachment sites 1105 can also comprise [3] surface site(s) configured to attach to a substrate 1101. In some cases, the plurality of attachment sites 1105 may further comprise [4] nanostructure attachment site(s). Nanostructure attachment site(s) on a first DNA nanostructure 1103 may be configured to bind to nanostructure attachment site(s) on a second (e.g., adjacent) DNA nanostructure 1103. In some cases, a DNA nanostructure may comprise one or more core nanoparticles (NP), each with a corresponding DNA origami shell. In some cases, a DNA nanostructure may not comprise a core nanoparticle. In some cases, a DNA nanostructure with multiple core nanoparticles and corresponding DNA origami shells may further comprise one or more intervening linkers, e.g., each DNA origami shell may be separated by one or more linkers as described elsewhere herein.

In some cases, the concentration of the supports and the template nucleic acids may be adjusted, such as to have a lower concentration of template nucleic acids than that of supports, to further the likelihood that a support couples to at most one template nucleic acid.

In some cases, the reaction kinetics of the support-to-template attachment may be accelerated by providing a diffusion-limiting or crowding agent, such as polyethylene glycol (PEG) or other polymer (e.g., non-reactive polymer). The polymer or PEG may be provided at any useful molar mass, e.g., PEG 100 (g/mol), PEG 200, PEG 300, PEG 400, PEG 500, PEG 600, PEG 700, PEG 800, PEG 900, PEG 1000, PEG 2000, PEG 3000, PEG 4000, PEG 5000, PEG 6000, PEG 7000, PEG 8000, PEG 9000, PEG 10000, or higher. The PEG may be provided at a range of molar masses, e.g., from about 4000 g/mol to about 8000 g/mol. The PEG or polymer may be provided at any useful concentration, for example at least and/or at most about 0.1% w/v, 1% w/v, 2% w/v, 3% w/v, 4% w/v, 5% w/v, 6% w/v, 7% w/v, 8% w/v, 9% w/v, 10% w/v, 20% w/v, 30% w/v, 40% w/v, 50% w/v, 60% w/v, 70% w/v, 80% w/v, 90% w/v, or greater. The diffusion-limiting agent may increase the likelihood of template nucleic acids contacting the rare pre-enrichment site(s) on the support.

Also provided herein are methods for loading supports onto a substrate, where sequencing is performed while the support is immobilized to the substrate. In some cases, the supports may be loaded after amplification on the supports, such that amplified supports are dispensed and immobilized to the substrate. In some cases, the supports may be loaded prior to performing amplification on the supports, such that, for example, pre-enriched (e.g., single template-attached) or non-pre-enriched (no template-attached) supports are dispensed and immobilized to the substrate, and then subject to amplification on the substrate. A method for loading supports onto a substrate may comprise contacting a plurality of supports to the substrate, wherein the plurality of supports comprises a plurality of DNA nanostructures, wherein the plurality of DNA nanostructures comprises amplification sites.

In some cases, the substrate may be unpatterned and/or untextured and the DNA nanostructures may self-assemble and immobilize onto the unpatterned substrate.

In some cases, the substrate (e.g., wafer) may be patterned and/or textured with binders, linkers, and/or active chemical groups as described elsewhere herein. The DNA nanostructures may comprise one or more surface attachment sites configured to bind to such binders, linkers, and/or active chemical groups. In one example, the binder on the substrate and surface site on the DNA nanostructure comprise a complementary oligonucleotide pair, click chemistry pair, and/or cross-link pair. In some cases, the substrate may comprise multiple types of binders configured to bind to different types of surface sites. In some cases, the DNA nanostructure may comprise multiple types of surface sites configured to bind to different types of binders. In some cases, a collection of DNA nanostructures may comprise multiple types of surface sites configured to bind to different types of binders. FIG. 11A illustrates example DNA nanostructures 1103 which comprise a [3] surface site. The surface site may be used to immobilize the DNA nanostructures to the substrate 1101, such as via binders on the substrate. In some cases, the substrate may be patterned and/or textured, and the DNA nanostructures may not comprise a surface site. For example, a DNA nanostructure may immobilize to an elevated ‘pad’ (comprising distinct surface chemistry) on the substrate via electrostatic interactions between the surface chemistry on the pad and nucleic acid molecule(s) attached to a DNA nanostructure. A [3] surface site may comprise a coupling moiety (e.g., azide moiety, DBCO moiety, thiol moiety, oligonucleotide sequence, a cross-linking base, etc.) capable of binding to the substrate.

FIG. 11A illustrates example DNA nanostructures 1103 which comprise multiple [4] nanostructure connection sites. Nanostructure connection sites may be used to immobilize the DNA nanostructures to each other (e.g., to stabilize a self-assembled layer of DNA nanostructures on a surface). In some cases, a DNA nanostructure may comprise one or more [3] surface sites and one or more [4] nanostructure connection sites. In some cases, a DNA nanostructure may comprise only one or more [3] surface sites. In some cases, a DNA nanostructure may comprise only one or more [4] nanostructure connection sites.

FIG. 11B illustrates another example of DNA nanostructures 1113. In the illustration, the DNA nanostructures 1113 comprise a few or single [1] amplification sites and many [2] pre-enrichment site(s). DNA nanostructures 1113 comprise one or more [3] surface sites. In addition, DNA nanostructures 1113 comprise [4] nanostructure connection sites. In some cases, nanostructure connection sites may be positioned randomly on DNA nanostructures. In some cases, nanostructure connection sites may be positioned at respective locations on DNA nanostructures. A nanostructure connection site may comprise a coupling moiety (e.g., azide moiety, DBCO moiety, thiol moiety, oligonucleotide sequence, a cross-linking base, etc.) capable of binding to another nanostructure connection site. That is, there may be sets of nanostructure connection sites comprising coupling pairs (e.g., azide and DBCO, or complementary oligonucleotide sequences). As shown in FIG. 11B, DNA nanostructures 1113 may comprise multiple pairs of nanostructure connection sites. That is, sites 1114 and 1116 are configured to couple to sites 1114′ and 1116′, respectively. By way of example, site 1114 may comprise a first oligonucleotide sequence, and site 1114′ may comprise a second oligonucleotide, wherein the second oligonucleotide sequence is complementary to the first oligonucleotide sequence; site 1116 may comprise a third oligonucleotide and site 1116′ may comprise a fourth oligonucleotide sequence, wherein the third and fourth oligonucleotide sequences are complementary. In some cases, the third sequence may be the same as the first sequence and the fourth sequence may be the same as the second sequence. Likewise, in DNA nanostructure 1113, sites 1110 and 1112 may be configured to couple to sites 1110′ and 1112′, respectively (e.g., as shown). By way of example, site 1110 may comprise an azide moiety and site 1110′ may comprise a DBCO moiety; sites 1112 and 1112′ may comprise thiols. In some cases, a DNA nanostructure may comprise one or more nanostructure connection sites comprising coupling moieties all of a same type (e.g., all oligonucleotides). In some cases, a DNA nanostructure may comprise one or more nanostructure connection sites comprising a first coupling moiety type (e.g., click-chemistry compatible such as DBCO or azide) and one or more nanostructure connection sites comprising a second coupling moiety type (e.g., thiol). In some cases, a plurality of DNA nanostructures loaded onto a substrate may all comprise a same set of nanostructure connection sites (e.g., comprising thiol moieties). In some cases a plurality of DNA nanostructures loaded onto a substrate may comprise a first set of DNA nanostructures comprising a first set of nanostructure connection sites and a second set of DNA nanostructures comprises a second set of nanostructure connection sites. In this way, adjacent DNA nanostructures may be coupled to each other (e.g., after loading on a substrate 1101). Adjacent DNA nanostructures may be coupled to each other prior to, subsequent to, or concurrent with coupling to the substrate.

FIG. 11C illustrates several example shapes of DNA nanostructures. Nanostructure 1121 comprises a triangular pyramid (e.g., a DNA origami nanostructure in an approximately triangular pyramidal shape) comprising a [2] pre-enrichment site at one apex, [4] nanostructure connection sites at the other three apexes, and a [3] surface site on the base. Nanostructure 1123 comprises a square pyramid (e.g., a DNA origami nanostructure in an approximately rectangular pyramidal shape) comprising a [2] pre-enrichment site at one apex, [4] nanostructure connection sites at three other three apexes, and a [3] surface site on the remaining apex. Nanostructure 1125 comprises a DNA nanoball comprising a [2] pre-enrichment site at one end of the concatemer, a [3] surface site at the other end of the concatemer, and a plurality of [4] nanostructure connection sites attached throughout the concatemer (e.g., where the nanostructure connection sites are coupled to the nanoball via oligonucleotides 1127 with complementarity to regions of the nanoball). Nanostructure 1129 comprises a DNA nanoball comprising a [2] pre-enrichment site at one end of the concatemer, a [3] surface site at the other end of the concatemer, a plurality of [4] nanostructure connection sites attached throughout the concatemer (where the nanostructure connection sites may be coupled to the nanoball via oligonucleotides 1127 that are complementary to first regions of the nanoball), and one or more [1] amplification sites (where the amplification sites may be coupled to the nanoball via oligonucleotides 1131 that are complementary to second regions of the nanoball, where first and second regions may comprise the same sequence or where first and second regions may comprise different sequences).

The DNA nanostructures may comprise a plurality of sites comprising a pre-enrichment site and an amplification site, where a template nucleic acid is able to bind to a pre-enrichment site but not an amplification site, as described elsewhere herein. In some cases, a pre-enrichment site may comprise a sequencing site. For example, the DNA nanostructures may comprise a sequencing site without amplification sites, where a template molecule (e.g., a single template molecule (e.g., for single molecule sequencing) or a concatemer such as a DNA nanoball) is able to bind. The DNA nanostructures may comprise an amplification site without a pre-enrichment site, where a template nucleic acid is able to bind to any amplification site. In such a configuration, for example, each amplification site may comprise an oligonucleotide comprising a first sequence comprising an amplification primer and a second sequence comprising a capture sequence complementary to an adapter sequence of the template nucleic acid.

The DNA nanostructures provided to the substrate may be pre-enriched, i.e., attached to at least one template nucleic acid or a single template nucleic acid, such as to the pre-enrichment site or the amplification site. After pre-enriched DNA nanostructures are loaded to the substrate, the support-template complexes may be subjected to amplification (e.g., using any of the amplification methods described herein) on the surface of the substrate, to generate amplified supports. The amplified supports may be subjected to sequencing while immobilized to the substrate.

The DNA nanostructures provided to the substrate may be non-pre-enriched, i.e., not attached to at least one template nucleic acid. After non-pre-enriched DNA nanostructures are loaded to the substrate, template nucleic acids may be provided to contact the supports and generate support-template complexes on the surface of the substrate. In some cases, the DNA nanostructures may comprise a pre-enrichment site and amplification site as described elsewhere herein to achieve single-template pre-enrichment of at least a subset of the supports on the surface. In other cases, the DNA nanostructures may comprise amplification sites and no pre-enrichment site and template nucleic acids may be provided at low concentration, for example, to achieve single-template pre-enrichment of at least a subset of the supports on the surface. The support-template complexes may be subjected to amplification (e.g., using any of the amplification methods described herein) on the surface of the substrate, to generate amplified supports. The amplified supports may be subjected to sequencing while immobilized to the substrate.

In other instances, the DNA nanostructures provided to the substrate may be conducive for single molecule sequencing. For example, the DNA nanostructures may comprise a template attachment site but not comprise any amplification site. For example, a pre-enrichment site as described herein may function as a template attachment site. The template may be pre-attached to the DNA nanostructure prior to loading the DNA nanostructures onto the substrate. Alternatively, the DNA nanostructures may be pre-loaded onto the substrate and the templates deposited onto the DNA nanostructures to bind the template to the template attachment sites. Beneficially, the DNA nanostructures may be used to space out single molecule templates. A single molecule template may be non-concatemeric. A single molecule template may be concatemeric.

Provided herein are systems, compositions, and kits that comprise a DNA nanostructure as described herein. The DNA nanostructure can comprise a plurality of attachment sites. The DNA nanostructure can comprise a pre-enrichment site, an amplification site, and/or a surface site. The systems, compositions, and kits may further comprise template nucleic acids and/or amplified derivatives thereof attached to the DNA nanostructure. The systems, compositions, and kits may comprise a plurality of DNA nanostructures. systems, compositions, and kits may comprise a substrate.

### Substrate Coating

A plurality of polymers and/or dendrimers may be dispensed and immobilized on the substrate, wherein all or a subset of the polymers and/or dendrimers comprises attachment sites, such as described with respect to DNA nanostructures herein. For example, the polymer may comprise PEG, as described elsewhere herein. In some cases, a functionalized surface of the substrate may comprise the plurality of polymers and/or dendrimers. The plurality of polymers and/or dendrimers may function as attachment sites for supports (e.g., beads or DNA nanostructures or a combination thereof). Alternatively or in addition, the plurality of polymers and/or dendrimers may function as attachment sites for template nucleic acids and/or their derivatives (e.g., amplified products). For example, the attachment sites may be strategically placed amongst the polymers and/or dendrimers and/or amongst a subset of the polymers and/or dendrimers.

### DNA Nanoballs and Beads for Loading

Provided herein are devices, systems, methods, compositions, and kits that use DNA nanoballs and/or beads to facilitate loading of nucleic acids for sequencing on a substrate. For example, DNA nanoballs and/or beads may be used as supports to immobilize nucleic acids, which supports are immobilized to a substrate. In another example, DNA nanoballs and/or beads may be used as spacing and/or self-assembling objects that are used to space out and/or self-assemble nucleic acids on the substrate. Such devices, systems, methods, compositions, and kits can be applied alternatively or in addition to the sequencing workflow 100 of FIG. 1. Such devices, systems, methods, compositions, and kits can be used in conjunction with the sample processing systems and methods, or components thereof (e.g., substrates, detectors, reagent dispensing, continuous scanning, etc.) described herein.

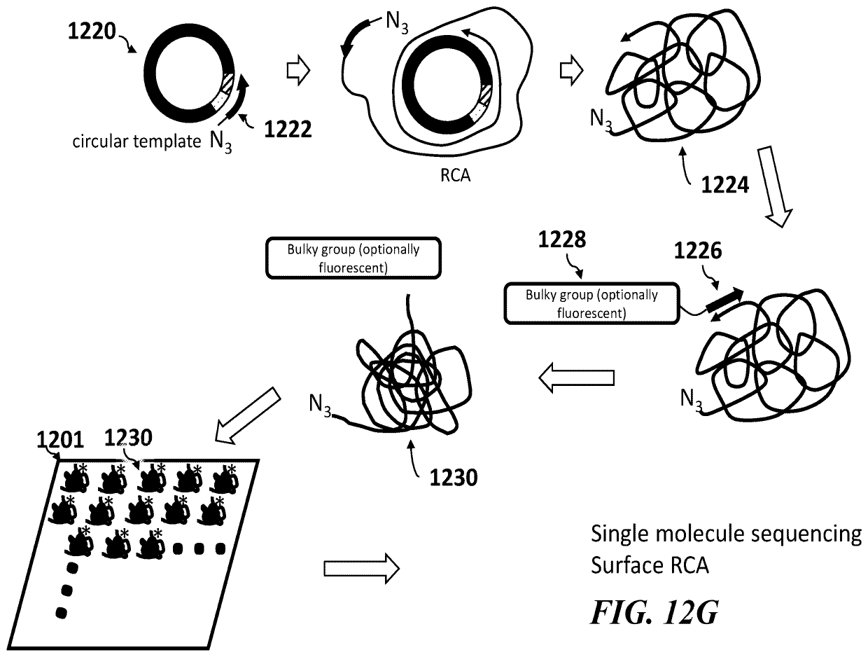

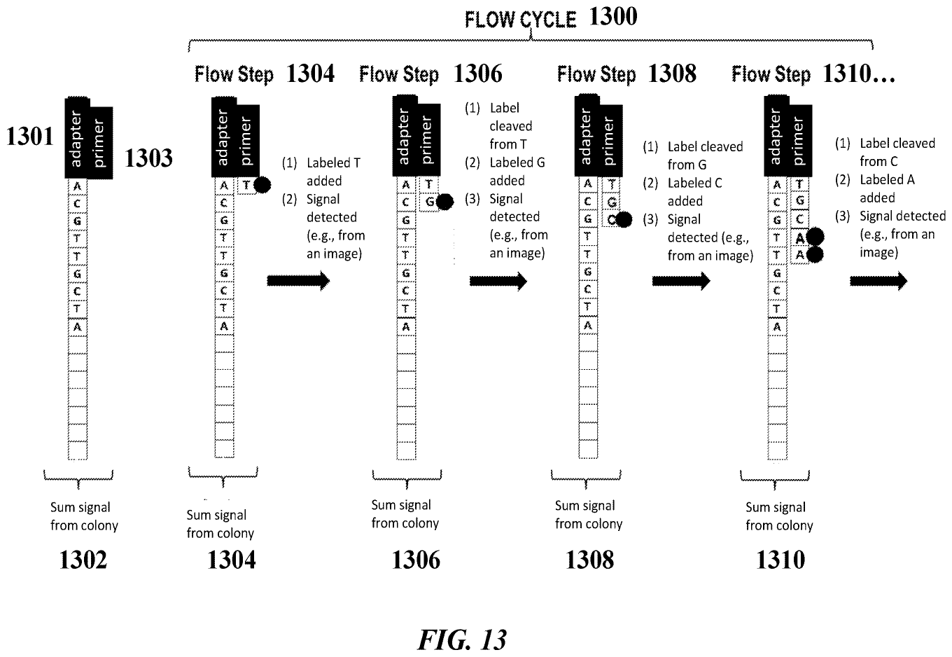

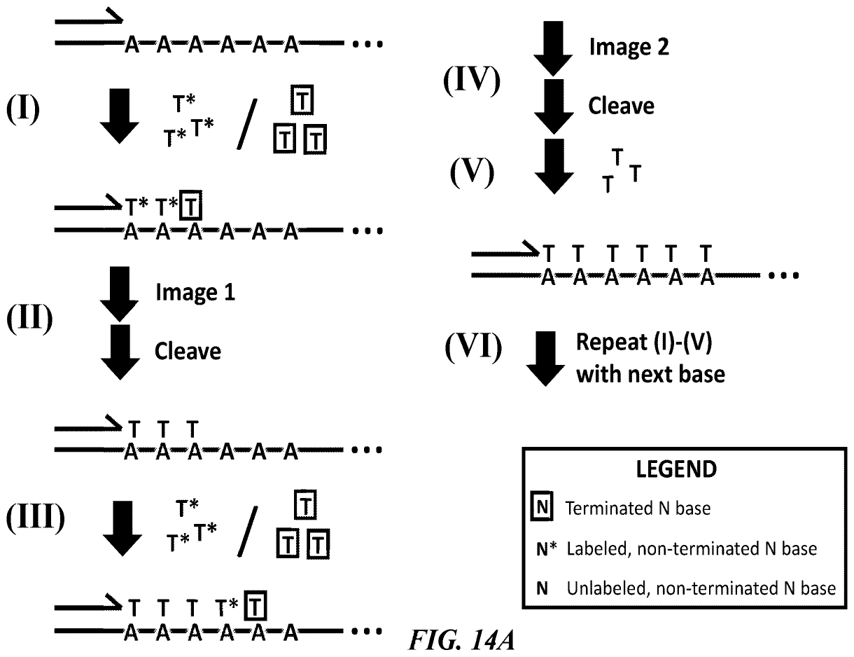

Nucleic acids may be loaded onto a substrate using beads, DNA nanostructures (e.g., origami), DNA nanoballs, or a combination thereof. FIG. 12A-12C illustrate different workflows for loading nucleic acids using beads as spacers. As shown in FIG. 12A, pre-enriched template-bead assemblies (or positive beads) may be loaded onto a substrate such that a template of a given template-bead assembly binds to the substrate. Pre-enrichment may refer to the generation of template-bead assemblies via contacting templates and beads together and then isolation of template-bead assemblies from other templates and beads that did not attach to each other. Pre-enriched template-bead assemblies may refer to the isolated template-bead assembly population. The substrate may be patterned or unpatterned. For example, the substrate may be patterned with binders that are configured to bind to templates of template-bead assemblies. In some cases, the substrate may be patterned or coated with DNA nanostructures as described elsewhere herein, where the DNA nanostructures are configured to bind to beads of template-bead assemblies. In another example, the substrate may be unpatterned such that there is a substantially uniform coating of a surface chemistry on the substrate. The surface chemistry may comprise binders that are configured to bind to templates. For example, the surface chemistry may comprise DBCO moieties, and the template may comprise azide moieties, respectively, which can couple together (e.g., template to surface) via click chemistry. Any one or more coupling mechanisms described elsewhere herein may be used for the template-substrate binding, such as any click chemistry pair, complementary oligonucleotides that hybridize, magnetic particles that are forced together by magnetic fields, electric particles that are forced together by electric fields, specific binding, non-specific binding, electrostatic interactions, cross-linking, etc. The substrate and/or template may comprise any binder described elsewhere herein. For example, the template may comprise a first coupler of a coupling pair and the substrate may comprise a second coupler of the coupling pair. In some cases, a single template may comprise a single moiety (e.g., azide moiety, DBCO moiety, thiol moiety, oligonucleotide sequence, a cross-linking base, etc.) capable of binding to the substrate. In another example, the template may have a negative charge and a surface chemistry of the substrate may have a positive charge which is electrostatically attracted to the negative charge. In some cases, a single template may comprise a plurality of moieties (e.g., at the same location or different locations on template strand(s)) capable of binding to the substrate. A single moiety, any subset of moieties, or all of the plurality of moieties may be used to bind to the substrate. In some cases, a single binder on the substrate may bind to a single template. In some cases, multiple binders on the substrate may bind to a single template. In some cases, a template may be bound at one end to the bead and at the other end to the substrate. In other cases, a template may be bound to the bead and/or the substrate at a location that is not at the 5′ or 3′ end of a strand, for example at a base that is adjacent to a 5′ or 3′ end of a strand. Beneficially, the beads bound to the templates in the template-bead assemblies may function as spacers and/or self-assembling objects on the substrate which prevents one template from binding too close to another template on the substrate. For example, after loading, a template-to-template pitch (center-to-center distance) may be at least or about a bead-to-bead pitch (center-to-center distance) when the template-bead assemblies are loaded as a result of the spacing/self-assembling between the beads. In some cases, after loading, an average template-to-template pitch may be at least an average bead-to-bead pitch and/or at least an average bead diameter. In some cases, upon depositing the template-bead assemblies on the substrate, the template-bead assemblies may be permitted to self-assemble or space out on the substrate before a binding reaction between the template and the substrate is activated via one or more stimuli (e.g., chemical reagent, catalyst, light (e.g., UV light), heat, etc.) to bind the templates to the substrate. In other cases, the template-bead assemblies may be deposited on the substrate under conditions sufficient to permit binding of the template to the substrate upon contact. After spacing out and/or self-assembling, the beads may be cleaved or otherwise removed from the templates and washed away. The cleaving may occur before or after the templates bind to the substrate. The beads may be washed away after the templates bind to the substrate.

Alternatively, as shown in FIG. 12B, a mixture of non-pre-enriched template-bead assemblies (or positive beads) and negative beads (not bound to any templates) may be loaded onto a substrate such that a template of a given template-bead assembly binds to the substrate. In this case, the negative beads in the mixture are unable to bind to the substrate as they lack a template. Once the beads in the template-bead assemblies are cleaved and washed, the negative beads will also get washed away. In some cases, DNA nanostructures (e.g., DNA origami, DNA nanoballs, etc.) may be used instead of or in addition to negative beads. In some cases, after loading, an average template-to-template pitch may be at least an average bead-to-bead pitch and/or at least an average bead diameter. In some cases, upon depositing the template-bead assemblies on the substrate, the template-bead assemblies may be permitted to self-assemble or space out on the substrate before a binding reaction between the template and the substrate is activated via one or more stimuli (e.g., chemical reagent, catalyst, light (e.g., UV light), heat, etc.) to bind the templates to the substrate. In other cases, the template-bead assemblies may be deposited on the substrate under conditions sufficient to permit binding of the template to the substrate upon contact. After spacing out and/or self-assembling, the beads may be cleaved or otherwise removed from the templates and washed away. The cleaving may occur before or after the templates bind to the substrate. The beads may be washed away after the templates bind to the substrate.

Alternatively, as shown in FIG. 12C, template-bead assemblies may be loaded onto a substrate such that a bead of a given template-bead assembly binds to the substrate. The template-bead assemblies may be pre-enriched such that only positive beads (bound to a template) are deposited on the substrate. The template-bead assemblies may be non-pre-enriched such that a mixture of positive and negative beads are deposited on the substrate. The substrate may be patterned or unpatterned. For example, the substrate may be patterned with binders that are configured to bind to beads of template-bead assemblies. In some cases, the substrate may be patterned or coated with DNA nanostructures as described elsewhere herein, where the DNA nanostructures are configured to bind to beads of template-bead assemblies. In another example, the substrate may be unpatterned such that there is a substantially uniform coating of a surface chemistry on the substrate. The surface chemistry may comprise binders that are configured to bind to beads. Any one or more coupling mechanisms described elsewhere herein may be used for the bead-substrate binding, such as any click chemistry pair, complementary oligonucleotides that hybridize, magnetic particles that are forced by magnetic fields, electric particles that are forced by electric fields, specific binding, non-specific binding, electrostatic interactions, cross-linking, etc. The substrate and/or bead may comprise any binder described elsewhere herein. In some cases, the bead may comprise a plurality of primers that are not bound or extended into a template, which plurality of primers may be used to bind to the substrate. In some cases, the bead may comprise a first coupler of a coupling pair and the substrate may comprise a second coupler of the coupling pair. In some cases, a single bead may comprise a single moiety (e.g., azide moiety, DBCO moiety, thiol moiety, oligonucleotide sequence, a cross-linking base, etc.) capable of binding to the substrate. In another example, the bead or components attached thereto may have a negative charge and a surface chemistry of the substrate may have a positive charge which is electrostatically attracted to the negative charge. In some cases, a single bead may comprise a plurality of moieties (e.g., at the same location or different locations on template strand(s)) capable of binding to the substrate. A single moiety, any subset of moieties, or all of the plurality of moieties may be used to bind to the substrate. In some cases, a single binder on the substrate may bind to a single bead. In some cases, multiple binders on the substrate may bind to a single bead. In some cases, a template may be bound at one end to the bead. In other cases, a template may be bound to the bead at a location that is not at the 5′ or 3′ end of a strand, for example at a base that is adjacent to a 5′ or 3′ end of a strand. In this workflow, a template may not be directly bound to the substrate. Beneficially, the beads bound to the templates in the template-bead assemblies may function as spacers and/or self-assembling objects on the substrate which prevents one template from being immobilized too close to another template on the substrate. For example, after loading, a template-to-template pitch (center-to-center distance) may be at least a bead-to-bead pitch (center-to-center distance) when the template-bead assemblies are loaded as a result of the spacing/self-assembling between the beads. Where non-pre-enriched mixture of positive and negative beads are deposited, the average template-to-template pitch may be greater due to the presence of non-template-bound negative beads also loaded on the substrate. In some cases, after loading, an average template-to-template pitch may be at least an average bead-to-bead pitch and/or at least an average bead diameter. In some cases, upon depositing the template-bead assemblies on the substrate, the template-bead assemblies may be permitted to self-assemble or space out on the substrate before a binding reaction between the bead and the substrate is activated via one or more stimuli (e.g., chemical reagent, catalyst, light (e.g., UV light), heat, etc.) to bind the beads to the substrate. In other cases, the template-bead assemblies may be deposited on the substrate under conditions sufficient to permit binding of the bead to the substrate upon contact. After the beads are immobilized, in some cases, the beads may be subjected to shrinking conditions. Templates attached to the beads may be further spaced apart from neighboring templates via the shrinking as on average each template is pulled closer to the center of the bead in each template-bead assembly.

Nucleic acids may be loaded onto a substrate using DNA nanoballs. For example, in each of the workflows described with respect to FIG. 12A-12C, the beads may be replaced with DNA nanoballs. In some cases, a combination of beads and DNA nanoballs may be used to load nucleic acids onto a substrate. Alternatively, or in addition, in some cases, a combination of beads and DNA nanostructures (e.g., DNA nanoballs, DNA origami, or other DNA organizations) to load nucleic acids onto a substrate. For example, in some cases, a first plurality of templates may be assembled with DNA nanoballs and a second plurality of templates may be associated with beads. The first and second plurality of template assemblies may be loaded onto a substrate concurrently or sequentially. FIGS. 12D-12F illustrate different workflows for loading nucleic acids using DNA nanoballs as spacers.

As shown in FIG. 12D, template-nanoball assemblies may be loaded onto a substrate such that a template of a given template-nanoball assembly binds to the substrate. In some cases, nucleic acid nanostructures (e.g., DNA origami, or other organized nucleic acid structures), beads, or a combination thereof may be used instead of or in addition to nanoballs. The substrate may be patterned or unpatterned. For example, the substrate may be patterned with binders that are configured to bind to templates of template-nanoball assemblies. In some cases, the substrate may be patterned or coated with DNA nanostructures as described elsewhere herein, where the DNA nanostructures are configured to bind to templates of template-nanoball assemblies. In another example, the substrate may be unpatterned such that there is a substantially uniform coating of a surface chemistry on the substrate. The surface chemistry may comprise binders that are configured to bind to templates. For example, the surface chemistry may comprise DBCO or azide moieties, and the template may comprise azide moieties or DBCO moieties, respectively, which can couple together via click chemistry. Any one or more coupling mechanisms described elsewhere herein may be used for the template-substrate binding, such as any click chemistry pair, complementary oligonucleotides that hybridize, magnetic particles that are forced by magnetic fields, electric particles that are forced by electric fields, specific binding, non-specific binding, electrostatic interactions, cross-linking, etc. The substrate and/or template may comprise any binder described elsewhere herein. For example, the template may comprise a first coupler of a coupling pair and the substrate may comprise a second coupler of the coupling pair. In some cases, a single template may comprise a single moiety (e.g., azide moiety, DBCO moiety, thiol moiety, oligonucleotide sequence, a cross-linking base, etc.) capable of binding to the substrate. In another example, the template may have a negative charge and a surface chemistry of the substrate may have a positive charge which is electrostatically attracted to the negative charge. In some cases, a single template may comprise a plurality of moieties (e.g., at the same location or different locations on template strand(s)) capable of binding to the substrate. A single moiety, any subset of moieties, or all of the plurality of moieties may be used to bind to the substrate. In some cases, a single binder on the substrate may bind to a single template. In some cases, multiple binders on the substrate may bind to a single template. In some cases, a template may be bound at, or comprise at, one end to the nanoball and at the other end to the substrate. In other cases, a template may be bound to the nanoball and/or the substrate at a location that is not at the 5′ or 3′ end of a strand, for example at a base that is adjacent to a 5′ or 3′ end of a strand. Beneficially, the nanoballs bound to the templates in the template-nanoball assemblies may function as spacers and/or self-assembling objects on the substrate which prevents one template from binding too close to another template on the substrate. For example, after loading, a template-to-template pitch (center-to-center distance) may be at least a nanoball-to-nanoball pitch (center-to-center distance) when the template-nanoball assemblies are loaded as a result of the spacing/self-assembling between the nanoballs. In some cases, after loading, an average template-to-template pitch may be at least an average nanoball-to-nanoball pitch and/or at least an average nanoball diameter. In some cases, upon depositing the template-nanoball assemblies on the substrate, the template-nanoball assemblies may be permitted to self-assemble or space out on the substrate before a binding reaction between the template and the substrate is activated via one or more stimuli (e.g., chemical reagent, catalyst, light (e.g., UV light), heat, etc.) to bind the templates to the substrate. In other cases, the template-nanoball assemblies may be deposited on the substrate under conditions sufficient to permit binding of the template to the substrate upon contact. After spacing out and/or self-assembling, the nanoballs may be cleaved or otherwise removed from the templates and washed away. The cleaving may occur before or after the templates bind to the substrate. The nanoballs may be washed away after the templates bind to the substrate.

In some cases, instead of cleaving nanoballs, the nanoballs may be protected (e.g., may be double-stranded) and thus unavailable for sequencing. In such cases, after loading and binding of template-nanoball assemblies, the templates may be subjected to conditions sufficient for sequencing (e.g., where a sequencing primer may anneal to a template and not to nanoballs).

Alternatively, as shown in FIG. 12E, template-nanoball assemblies may be loaded onto a substrate such that a nanoball of a given template-nanoball assembly binds to the substrate. In some cases, nucleic acid nanostructures (e.g., DNA origami, or other organized nucleic acid structures), beads, or a combination thereof may be used instead of or in addition to nanoballs. The substrate may be patterned or unpatterned. For example, the substrate may be patterned with binders that are configured to bind to nanoballs of template-nanoball assemblies. In some cases, the substrate may be patterned or coated with DNA nanostructures as described elsewhere herein, where the DNA nanostructures are configured to bind to nanoballs of template-nanoball assemblies. In another example, the substrate may be unpatterned such that there is a substantially uniform coating of a surface chemistry on the substrate. The surface chemistry may comprise binders that are configured to bind to nanoballs. Any one or more coupling mechanisms described elsewhere herein may be used for the nanoball-substrate binding, such as any click chemistry pair, complementary oligonucleotides that hybridize, magnetic particles that are forced by magnetic fields, electric particles that are forced by electric fields, specific binding, non-specific binding, electrostatic interactions, cross-linking, etc. The substrate and/or nanoball may comprise any binder described elsewhere herein. In some cases, the nanoball may comprise a first coupler of a coupling pair and the substrate may comprise a second coupler of the coupling pair. In some cases, a single nanoball may comprise a single moiety (e.g., azide moiety, DBCO moiety, thiol moiety, oligonucleotide sequence, a cross-linking base, etc.) capable of binding to the substrate. In another example, the nanoball may have a negative charge and a surface chemistry of the substrate may have a positive charge which is electrostatically attracted to the negative charge. In some cases, a single nanoball may comprise a plurality of moieties (e.g., at the same location or different locations on template strand(s)) capable of binding to the substrate. A single moiety, any subset of moieties, or all of the plurality of moieties may be used to bind to the substrate. In some cases, a single binder on the substrate may bind to a single nanoball. In some cases, multiple binders on the substrate may bind to a single nanoball. In some cases, a template may be bound at one end to the nanoball. In other cases, a template may be bound to the nanoball at a location that is not at the 5′ or 3′ end of a strand, for example at a base that is adjacent to a 5′ or 3′ end of a strand. In this workflow, a template may not be directly bound to the substrate. Beneficially, the nanoballs bound to the templates in the template-nanoball assemblies may function as spacers and/or self-assembling objects on the substrate which prevents one template from being immobilized too close to another template on the substrate. For example, after loading, a template-to-template pitch (center-to-center distance) may be at least a nanoball-to-nanoball pitch (center-to-center distance) when the template-nanoball assemblies are loaded as a result of the spacing/self-assembling between the nanoballs. In some cases, after loading, an average template-to-template pitch may be at least an average nanoball-to-nanoball pitch and/or at least an average nanoball diameter. In some cases, upon depositing the template-nanoball assemblies on the substrate, the template-nanoball assemblies may be permitted to self-assemble or space out on the substrate before a binding reaction between the nanoball and the substrate is activated via one or more stimuli (e.g., chemical reagent, catalyst, light (e.g., UV light), heat, etc.) to bind the nanoballs to the substrate. In other cases, the template-nanoball assemblies may be deposited on the substrate under conditions sufficient to permit binding of the nanoball to the substrate upon contact. After the nanoballs are immobilized, in some cases, the nanoballs may be subjected to shrinking conditions. Templates attached to the nanoballs may be further spaced apart from neighboring templates via the shrinking as on average each template is pulled closer to the center of the nanoball in each template-nanoball assembly.

In some cases, empty nanoballs or negative nanoballs not bound to any template may be co-deposited onto the substrate with the template-nanoball assemblies. The presence of additional negative nanoballs between template-nanoball assemblies may additionally space out the templates and increase the average template-to-template pitch (center-to-center distance).

Alternatively, as shown in FIG. 12F, empty nanoballs not bound to templates may be loaded onto a substrate such that the nanoballs bind to the substrate, and then templates may be deposited onto the nanoball-bound substrate to bind templates to the nanoballs. In some cases, nucleic acid nanostructures (e.g., DNA origami, or other organized nucleic acid structures), beads, or a combination thereof may be used instead of or in addition to nanoballs. Unbound nanoballs may be washed away before depositing the templates. The substrate may be patterned or unpatterned. For example, the substrate may be patterned with binders that are configured to bind to nanoballs. In some cases, the substrate may be patterned or coated with DNA nanostructures as described elsewhere herein, where the DNA nanostructures are configured to bind to nanoballs. In another example, the substrate may be unpatterned such that there is a substantially uniform coating of a surface chemistry on the substrate. The surface chemistry may comprise binders that are configured to bind to nanoballs. Any one or more coupling mechanisms described elsewhere herein may be used for the nanoball-substrate binding, such as any click chemistry pair, complementary oligonucleotides that hybridize, magnetic particles that are forced by magnetic fields, electric particles that are forced by electric fields, specific binding, non-specific binding, electrostatic interactions, cross-linking, etc. The substrate and/or nanoball may comprise any binder described elsewhere herein. In some cases, the nanoball may comprise a first coupler of a coupling pair and the substrate may comprise a second coupler of the coupling pair. In some cases, a single nanoball may comprise a single moiety (e.g., azide moiety, DBCO moiety, thiol moiety, oligonucleotide sequence, a cross-linking base, etc.) capable of binding to the substrate. In another example, the nanoball may have a negative charge and a surface chemistry of the substrate may have a positive charge which is electrostatically attracted to the negative charge. In some cases, a single nanoball may comprise a plurality of moieties (e.g., at the same location or different locations on template strand(s)) capable of binding to the substrate. A single moiety, any subset of moieties, or all of the plurality of moieties may be used to bind to the substrate. In some cases, a single binder on the substrate may bind to a single nanoball. In some cases, multiple binders on the substrate may bind to a single nanoball. In some cases, a single nanoball may comprise a single moiety (e.g., azide moiety, DBCO moiety, thiol moiety, oligonucleotide sequence, a cross-linking base, etc.) capable of binding to the template. In some cases, a single nanoball may comprise a plurality of moieties (e.g., at the same location or different locations on template strand(s)) capable of binding to the template. A single moiety, any subset of moieties, or all of the plurality of moieties may be used to bind to the template. In some cases, a single binder on the template may bind to a single nanoball. In some cases, multiple binders on the template may bind to a single nanoball. In some cases, a template may be bound at one end to the nanoball. In other cases, a template may be bound to the nanoball at a location that is not at the 5′ or 3′ end of a strand, for example at a base that is adjacent to a 5′ or 3′ end of a strand. In this workflow, a template may not be directly bound to the substrate. In some cases, each nanoball may bind to at most template. In some cases, each nanoball may bind at most 10, 9, 8, 7, 6, 5, 4, 3, 2, or 1 template. Beneficially, the nanoballs bound to the templates may function as spacers and/or self-assembling objects on the substrate which prevents one template from being immobilized too close to another template on the substrate. For example, after loading the templates, a template-to-template pitch (center-to-center distance) may be at least a nanoball-to-nanoball pitch (center-to-center distance) as a result of the spacing/self-assembling between the nanoballs. In some cases, after loading the templates, an average template-to-template pitch may be at least an average nanoball-to-nanoball pitch and/or at least an average nanoball diameter. In some cases, upon depositing the nanoballs on the substrate, the nanoballs may be permitted to self-assemble or space out on the substrate before a binding reaction between the nanoball and the substrate is activated via one or more stimuli (e.g., chemical reagent, catalyst, light (e.g., UV light), heat, etc.) to bind the nanoballs to the substrate. In other cases, the nanoballs may be deposited on the substrate under conditions sufficient to permit binding of the nanoball to the substrate upon contact. Before or after the templates are bound to the nanoballs, in some cases, the nanoballs may be subjected to shrinking conditions. Templates attached to the nanoballs may be further spaced apart from neighboring templates via the shrinking as on average each template or template binding site is pulled closer to the center of the nanoball in each template-nanoball assembly.

Beneficially, the loading mechanisms described herein may immobilize templates in a spaced-apart manner which enables spatial discerning of signals collected from individual templates immobilized to the substrate during sequencing reactions, such as single molecule sequencing reactions or concatemer sequencing reactions. A template may be a concatemer molecule or a non-concatemer molecule. The spacing apart may also reduce inter-dye effects, such as quenching or FRET, between dyes coupled to different templates that may affect sequencing quality.

It will be appreciated that another particle, support, or object may be used in place of nanoballs and beads in these workflows, such as a DNA origami particle, or non-DNA objects, such as nanoparticles. In some cases, any combination of particles, supports, objects, beads, DNA nanostructures, DNA origami, DNA nanoballs may be used in these workflows.

Different conditions and methods for shrinking beads, which may be applied generally to nanoballs and other objects (e.g., DNA origami particle, nanoparticles, etc.), are described in further detail in U.S. Patent Pub. No. 2023/0340570A1 and International Pub. No. 2023/069648A1, each of which is incorporated by reference herein in its entirety for all purposes. For example, particles (e.g., nanoballs, beads, etc.) may be subjected to incubation with a buffer solution comprising a polymer, such as polyethylene glycol (PEG), and/or a cation, such as a divalent cation, to shrink them. The substrate may be subjected to one or more washing operations, such as before, during, or after shrinking the particles.

In some instances, the cations may be magnesium ions, calcium ions, or spermine ions (e.g., spermine¹⁺, spermine²⁺, spermine³⁺, spermine⁴⁺, etc.), or a combination thereof. In some cases, the cations may comprise ions of aluminum, barium, bismuth, cadmium, calcium, cesium, chromium, cobalt, copper, copper, hydrogen, iron, iron, lead, lithium, magnesium, mercury, mercury, nickel, potassium, rubidium, silver, sodium, strontium, tin, or spermine. In some cases, the cations may comprise Al3+, Ba2+, Bi3+, Cd2+, Ca1+, Ca2+, Cs1+, CrH, Co2+, Cu1+, Cu2+, H1+, Fe2+, Fe3+, Pb2+, Li1+, Mg1+, Mg2+, Hg22+, Hg2+, Ni2+, K1+, Rb1+, Ag1+, Na1+, Sr2+, Sn2+, spermine1+, spermine2+, spermine3+, or spermine4+. The cation may facilitate shrinking of particle (e.g., beads, nanoballs, etc.) sizes. The substrate may be treated with a cation buffer solution to facilitate shrinking of particles. In some cases, a cation buffer solution may comprise about, at least about, and/or at most about 5 mM, 10 mM, 15 mM, 20 mM, 25 mM, 30 mM, 35 mM, 40 mM, about 45 mM, 50 mM, 55 mM, 60 mM, 65 mM, 70 mM, 75 mM, 80 mM, 85 mM, 90 mM, 95 mM, or 100 mM of cations. In some cases, the substrate may be treated with a PEG solution. The PEG molecule in the solution may have a molecular mass of up to about 100, 200, 300, 400, 500, 1000, 1500, 2000, 2500, 3000, 3500, 4000, 4500, 5000, 5500, 6000, 6500, 7000, 7500, 8000, 8500, 9000, 9500, 10000, 10500, 11000, 11500, 12000, 12500, 13000, 13500, 14000, 14500, 15000, 15500, 16000, 16500, 17000, 17500, 18000, 18500, 19000, 19500, 20000, or more Da. In some cases, a PEG molecule may have a molecular mass of more than about 20,000 Da. In some cases, a PEG molecule may have a molecular mass of less than about 100 Da. In some instances, a PEG molecule may have a molecular mass within a range defined by any two of the preceding values. In some cases, a PEG molecule may have a molecular weight of at least about 1×10⁴, 2×10⁴, 5×10⁴, 1×10⁵, 2×10⁵, 5×10⁵, 1×10⁶, 2×10⁶, 5×10⁶, 1×10⁷, 2×10⁷, 5×10⁷, 1×10⁸ or more grams per molecule (g/mol). In some cases, a PEG molecule may have a molecular weight of more than about 1×10⁸ g/mol. In some cases, a PEG molecule may have a molecular weight of less than about 1×10⁴ g/mol. In some cases, a PEG molecule may have a molecular weight within a range defined by any two of the preceding values. In some instances, the PEG concentration may be up to about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 15%, 20%, 25%, 30%, 35%, 40%, 45%, 50%, by weight, of a buffer solution. In some cases, the PEG concentration may be less than about 0.1% by weight, of a buffer solution. In some cases, the PEG concentration may be more than about 50% by weight, of a buffer solution. In some instances, the PEG concentration may be a percent by weight of a buffer solution within a range defined by any two of the preceding values. In some cases, the PEG concentration may be up to about 0.1%, 0.2%, 0.3%, 0.4%, 0.5%, 0.6%, 0.7%, 0.8%, 0.9%1, 1%, 2%, 3%, 4%, 5%, 6%, 7%, 8%, 9%, 10%, 15%, 20%, 25%, 30%, 35%, 40%, 45%, 50%, by volume, of a buffer solution. In some cases, the PEG concentration may be less than about 0.1%, by volume, of a buffer solution. In some cases, the PEG concentration may be more than about 50%, by volume, of a buffer solution. In some instances, the PEG concentration may be a percent by volume of a buffer solution within a range defined by any two of the preceding values.

In some instances, a layer of buffer solution comprising a cation and/or polymer molecule formed on the substrate may have a thickness of from about 1 nm to about 10 nm, from about 1 nm to about 100 nm, from about 1 nm to about 1 μm, from about 1 nm to about 10 μm, from about 1 nm to about 100 μm, or from about 1 nm to about 1 mm. In some cases, a layer of buffer solution formed on the substrate may have a thickness about 1 μm to about 40 μm, from about 1 μm to about 39 μm, from about 2 μm to about 38 μm, from about 3 μm to about 37 μm, from about 4 μm to about 36 μm, from about 5 μm to about 35 μm, from about 6 μm to about 34 μm, from about 7 μm to about 33 μm, from about 8 μm to about 32 μm, from about 9 μm to about 31 μm, from about 10 μm to about 30 μm, from about 11 μm to about 29 μm, from about 12 μm to about 28 μm, from about 13 μm to about 27 μm, from about 14 μm to about 26 μm, from about 15 μm to about 25 μm, from about 16 μm to about 24 μm, from about 17 μm to about 23 μm, from about 18 μm to about 22 μm, from about 19 μm to about 21 μm, from about 1 μm to about 20 μm, from about 5 μm to about 20 μm, from about 10 μm to about 20 μm, from about 15 μm to about 20 μm, from about 10 μm to about 25 μm, from about 10 μm to about 30 μm, from about 10 μm to about 35 μm, from about 10 μm to about 40 μm, from about 10 μm to about 20 μm, from about 10 μm to about 25 μm, from about 10 μm to about 30 μm, from about 10 μm to about 35 μm, from about 10 μm to about 40 μm, from about 5 μm to about 20 μm, from about 4 μm to about 20 μm, from about 3 μm to about 20 μm, from about 2 μm to about 20 μm, or from about 1 μm to about 20 μm. In some instances, a layer of buffer solution formed on the substrate may have a thickness of at least about 0.1 nm, 0.2 nm, 0.5 nm, 1 nm, 2 nm, 5 nm, 10 nm, 20 nm, 50 nm, 100 nm, 200 nm, 500 nm, 1 μm, 2 μm, 5 μm, 10 μm, 20 μm, 50 μm, 100 μm, 200 μm, 500 μm, 1 mm, or more than at least about 1 mm.

In some instances, an average size of a plurality of particles is measured in full-width at half-maximum (FWHM). As used herein, the term “FWHM” refers to a size (e.g., a diameter) of a particle determined from fluorescence imaging. In some instances, FWHM is the width of an intensity profile for the imaged particle, measured at the median intensity value (e.g., amplitude) detected from the particle (e.g., from an intensity profile of the fluorescence emitted from the particle). For instance, the FWHM may be determined for one or more particles in the plurality of particles, and an average size may be determined by averaging the one or more FWHM values so determined. In some instances, an intensity line profile corresponding to a respective particle is extracted from an image of the substrate. In some such instances, the FWHM for the particle is measured directly from the intensity line profile. In some such instances, the FWHM for the particle is estimated by fitting a Gaussian to the intensity line profile. In some instances, the FWHM for the particle is determined from a gray value version of the line intensity profile of the particle. In some instances, a FWHM may be determined for a particle at multiple time points (e.g., prior to, upon, and/or subsequent to a washing operation). In some instances, an average FWHM of a plurality of particles prior to subsequent to shrinking may be about, at least about, and/or at most about 0.1 μm, 0.5 μm, 1 μm, 5 μm, 10 μm, 50 μm, 100 μm, 500 μm, 1000 μm or 1 nm, 5 nm, 10 nm, 50 nm, 100 nm, 500 nm, 1000 nm or 1 μm, 5 μm, 10 μm, 50 μm, 100 μm, 500 μm, 1000 μm or 1 mm, or more. In some cases, the average FWHM of a plurality of particles may shrink by about, at least about, and/or at most about 1%, 2%, 3%, 4%, 5%, 6%, 7%, 8%, 9%, 1%, %15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% or more.

In some cases, DNA particles such as DNA nanoballs and DNA origami particles may be shrunk via applying staples that bind to different strand segments, thus reducing segment-to-segment distance(s) within a molecule and providing a reduced size form of the particle. In some cases, particles may be subjected to shrinking via other intra-molecular or inter-molecular linking mechanisms. In one example, a DNA particle may comprise thiol moieties which may link with each other to form disulfide bonds or link with an intermediary molecule that comprises thiol moieties to form disulfide bonds that reduces segment-to-segment distance(s) within a molecule to provide a reduced size form of the particle. In another example, a DNA particle may comprise cross-linkable bases (e.g., CNVK) that may link with other bases within the molecule or link with bases of an intermediary molecule to reduce segment-to-segment distance(s) within a molecule to provide a reduced size form of a particle. In another example, a DNA particle may comprise ‘click’able moieties that may link via click chemistry with each other or with an intermediary molecule that comprises complementary ‘click’able moieties. Accordingly, methods provided herein may comprise generating particles with linking reagents (e.g., a modified base comprising a thiol group, a modified base comprising a click chemistry group, a modified base that is cross-linkable, an oligonucleotide sequence that binds to a staple, an oligonucleotide sequence that binds to another intramolecular oligonucleotide sequence, etc.). For example, such linking reagents may be incorporated during synthesis of a DNA nanoball or DNA origami particles. Alternatively or in addition, a starting material, such as a primer that hybridizes to a circular template or an origami scaffold may comprise the linking reagent. The linking may be readily activatable, such as by providing one or more stimuli (e.g., providing staple reagents, providing light for crosslinking reaction, etc.). The linking may be reversible. The linking may be irreversible.

In some cases, a template-nanoball assembly, as described in FIGS. 12D-12F, may be generated by coupling a template to a nanoball. In some cases, the nanoball may be generated via a primer hybridized to and extending using a circular template, the primer comprising a template-binding moiety. The template-binding moiety may comprise any coupling mechanism described elsewhere herein. Thus, a nanoball generated from the primer comprises the template-binding moiety which can bind to the template. In some cases, the nanoball may be generated via a primer hybridized to and extending using a circular template, the primer being an extension from and/or being coupled to the template. The primer may be covalently or non-covalently coupled to the template. Thus, a nanoball generated from the primer comprises at one end the template and at the other end a concatemeric nanoball that is based off the circular template.

FIG. 12G illustrates another example of DNA nanoball loading onto a substrate. In some cases, this may be suitable for single molecule sequencing and/or surface RCA (e.g., RCA amplification of a nanoball-bound template). A nanoball source (e.g., a circular template, a circular non-template, circularized DNA, plasmid, etc.) 1220 may be amplified with a primer 1222, where the primer comprises a first coupling moiety (i.e., here an azide moiety) where the first coupling moiety may be configured for coupling to a substrate (e.g., to a binder on a substrate surface) or to a template molecule (e.g., a template nucleic acid). Amplification may produce DNA nanoball 1224, which comprises the first coupling moiety. In some cases, nanoball 1224 may be suitable for any of the methods described elsewhere herein (e.g., with reference to FIGS. 12D-12F). In some cases, nanoball 1224 may be subjected to further processing. For example, nanoball 1224 may be contacted with a plurality of primers 1226 (e.g., where the primers are random hexamers or have sequence complementarity with adapters in the circularized DNA 1220). In some cases the primers may comprise a second coupling moiety (e.g., different from the first coupling moiety). In some cases, the primers may further comprise a bulky group (e.g., a protein, a nanoparticle, etc.). In some cases, the bulky group may further comprise a label (e.g., any label as described herein). In some cases, the bulky group may be a label (e.g., green fluorescent protein (GFP)). In some cases, the bulky group may have an average diameter larger than the nanoball. After contacting nanoball 1224 with primers 1226, nanoball 1224 may be subjected to conditions sufficient for amplification (e.g., to produce a double-stranded nanoball 1230). In some cases, the at least two strands of a double-stranded nanoball 1230 may be linked together. For example, the at least two strands may comprise cross-linkable bases (e.g., CNVK) that may link with other bases within the molecule or link with bases of an intermediary molecule to prevent denaturation of the nanoball. For example, nanoball 1230 may comprise ‘click’able moieties that may link via click chemistry with each other or with an intermediary molecule that comprises complementary ‘click’able moieties. The linking may be readily activatable, such as by providing one or more stimuli (e.g., providing light for crosslinking reaction, etc.). The linking may be reversible. The linking may be irreversible. In some cases, a plurality of double-stranded nanoballs 1230 may be further subjected to size selection (e.g., to select for nanoballs that are entirely or mostly double-stranded and/or to select for nanoballs that are coupled to one or more bulky groups or to a predetermined number of bulky groups). In some cases, size selection may not be performed. Double-stranded nanoballs 1230 may be loaded onto a substrate surface 1201. In some cases, the substrate surface may be patterned or unpatterned as described elsewhere herein. In some cases, the substrate surface may comprise any suitable binders as described elsewhere herein. Double-stranded nanoballs 1230 may be coupled to the substrate surface. In some cases, any kind of loading described herein may be used (e.g., click chemistry, chemical affinity etc.). Optionally, loaded nanoballs 1230 may be shrunk after loading (e.g., using any suitable shrinking method described herein). Optionally, after loading nanoballs 1230, substrate 1201 may be imaged to confirm loading and/or distribution of nanoballs 1230 (e.g., to ensure desired density of loading for downstream processing). Beneficially, the double-stranded nanoballs 1230 bound to the substrate may function as spacers and/or self-assembling objects on the substrate which prevents one nanoball from being immobilized too close to another nanoball. For example, after loading the double-stranded nanoballs, a nanoball-to-nanoball pitch (center-to-center distance) may be at least a bulky group-to-bulky-group pitch (center-to-center distance) or at least an average bulky group diameter as a result of the spacing/self-assembling between the double-stranded nanoballs. After loading, bulky groups 1228 may be cleaved from the nanoballs, e.g., using any suitable method as described herein, thereby leaving a double-stranded nanoball 1230 comprising a functional moiety (e.g., a coupling moiety). After cleavage, template molecules may be loaded onto substrate surface 1201, where templates can be coupled to the functional moieties. Beneficially, this method enables the loading of nanoballs at distinct individually addressable locations and the binding of a single template molecule to a single nanoball (e.g., by ensuring each nanoball comprises a single functional group suitable for binding to a template molecule). This can reduce the incidence of polyclonality during sequencing. Templates may be sequenced (e.g., single molecule sequencing) or may be amplified (e.g., via RCA) and then sequenced (e.g., colony sequencing).

### Sequencing Methods

During sequencing by synthesis, a sequencing primer may be hybridized to a template (e.g., to a primer binding site on the template) and extended in a stepwise manner by, in each extension step, contacting the complex with nucleotide reagents of known canonical base type(s). The extended or extending sequencing primer may also be referred to herein as a growing strand. An extension step may be a bright step (also referred to herein, in some cases, as labeled step, hot step, or detected step) or a dark step (also referred to herein, in some cases, as an unlabeled step, cold step, or undetected step). A sequencing method may comprise only bright steps. Alternatively, a sequencing method may comprise a mix of bright step(s) and dark step(s). For a bright step, the growing strand may be contacted with nucleotide reagents that include labeled nucleotides (of known canonical base type(s)) and signals indicative of incorporation of the labeled nucleotides, or lack thereof, may be detected to determine a base or sequence of the template. Alternatively or in addition, for a bright step, the growing strand may be contacted with a mixture of labeled and unlabeled nucleotide reagents. For a dark step, the growing strand may be contacted with solely unlabeled nucleotide reagents. Alternatively or in addition, for a dark step, the growing strand may be contacted with labeled nucleotide reagents and detection omitted. Sequencing data can be generated from the signals collected after one or more extension steps. A sequencing by synthesis method may comprise any number of bright steps and any number of dark steps. A sequencing by synthesis method may comprise any number of bright regions (consecutive bright steps) and any number of dark regions (consecutive dark steps). In some cases, the dark steps or dark regions may be used to accelerate or fast forward through certain regions of the template during sequencing. In some cases, the dark steps or dark regions may be advantageous to correct phasing problems.

Sequencing methods of the present disclosure may comprise flow-based sequencing, non-terminated sequencing, and/or terminated sequencing. Sequencing methods of the present disclosure may be applied to colony-based sequencing where template strands are provided in clusters, each cluster comprising copies of a single template strand, concatemer-based sequencing where template strands are provided as concatemers, each concatemer comprising multiple copies of a single template insert, or single molecule-based sequencing where template strands are provided as single molecules as opposed to colonies, clusters, or concatemers. For non-single molecule-based sequencing methods, multiple sequencing primers may be simultaneously bound to multiple primer binding sites across multiple copies of a template insert (in clusters or in a concatemer), extended in parallel, and provide synchronized and cumulative signals from the multiple copies at bright steps.

### Terminated Sequencing

In terminated sequencing methods, a bright step may comprise terminated nucleotides (e.g., reversibly terminated nucleotides). In some cases, a bright step may comprise a single nucleotide base type (e.g., A, C, G, T, U) or a mixture of nucleotide base types (e.g., 2, 3, 4, or more base types). A dark step may comprise terminated nucleotides, unterminated nucleotides, or a mixture thereof. A dark step may comprise a single nucleotide base type. Alternatively, a dark step may comprise a mixture of nucleotide base types. In an extension step comprising solely reversibly terminated nucleotides (e.g., and not unterminated nucleotides) at most a single nucleotide base may be incorporated into a growing strand. In an extension step comprising a mixture of reversibly terminated and unterminated nucleotides, more than one nucleotide base may be incorporated into a growing strand, the last incorporation being of a terminated nucleotide. In some cases, a sequencing method may comprise using one or more mixtures of terminated and non-terminated nucleotides.

### Non-Terminated Sequencing

Sequencing data can be generated using flow-based sequencing methods that include extending a primer bound to a template nucleic acid according to a pre-determined flow cycle and/or flow order where, in one or more flow positions, known canonical base type(s) of nucleotides (e.g., A, C, G, T, U) is accessible to the extending primer. At least some of the nucleotides may include a label, which labeled nucleotides upon incorporation into the extending primer renders a detectable signal. The resulting sequence by which nucleotides are incorporated into the extended primer is expected to be the reverse complement of the sequence of the template nucleic acid. A method for sequencing can comprise using a flow sequencing method that includes (1) extending a primer using labeled nucleotides in a flow, and (2) detecting the presence or absence of a labeled nucleotide incorporated into the extending primer to generate sequencing data. Flow sequencing methods may also be referred to as “natural sequencing-by-synthesis,” “mostly natural sequencing-by-synthesis,” or “nonterminated sequencing-by-synthesis” methods. Example methods are described in U.S. Pat. Nos. 8,772,473 and 11,459,609B2, each of which is incorporated by reference herein in its entirety.

In flow sequencing, iterative nucleotide flows are used to extend the primer hybridized to the template nucleic acid, with detection of incorporated nucleotides between one or more flows. The nucleotides may be, for example, nonterminating nucleotides such that more than one consecutive base can be incorporated into the extending primer strand if more than one consecutive complementary base (or homopolymer region) is present in the template strand. At least a portion of the nucleotides can be labeled so that incorporation can be detected. Generally, only a single nucleotide type is introduced in a flow, although two or three different types of nucleotides may be simultaneously introduced in certain embodiments. This methodology can be contrasted with sequencing methods that use a reversible terminator, where primer extension is stopped after extension of every single base before the terminator is reversed (e.g., by removing a 3′ blocking group) to allow incorporation of the next succeeding base.

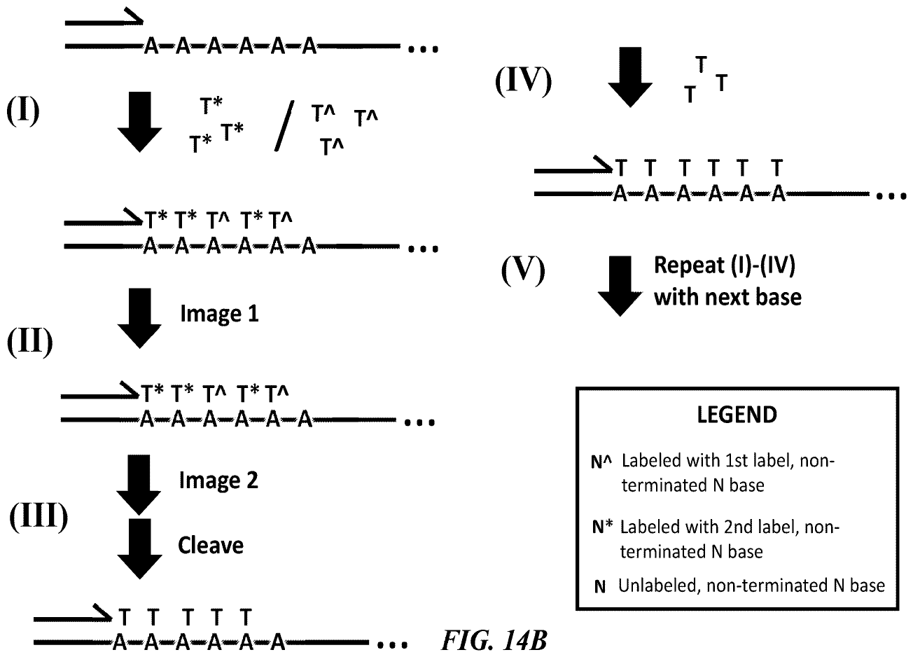

FIG. 13 illustrates an example flow sequencing method that can be used to generate the sequencing data described herein. Template nucleic acids may be immobilized to a surface (e.g., the surface of a bead attached to a substrate or directly to a substrate), as described in detail herein. In this example, the template nucleic acid includes an adapter sequence 1301 followed by an insert sequence (“ACGTTGCTA . . . ”). The adapter sequence 1301 can include a sequencing primer hybridization site. At operation 1302, a sequencing primer 1303 is hybridized to the adapter sequence 1301 at the sequencing primer hybridization site. The sequencing primer 1303 is then extended in a series of flows according to flow cycle 1300 with flow order: [T G C A]. In this example, the flow cycle 1300 includes four flow steps 1304, 1306, 1308, 1310, and in a given flow step, a single base type is provided to the template-primer hybrid. In flow step 1304, nucleotides comprising labeled T nucleotides are provided; in flow step 1306, nucleotides comprising labeled G nucleotides are provided; in flow step 1308, nucleotides comprising labeled C nucleotides are provided; in flow step 1310, nucleotides comprising labeled A nucleotides are provided. Nucleotides in a single-base flow may comprise a mixture of labeled and unlabeled nucleotides of the single base. At flow step 1304, a labeled T nucleotide is incorporated by the extending sequencing primer 1303 opposite the A base in the template strand. Then, a signal indicative of the incorporation of the labeled T nucleotide can be detected. For example, the signal may be detected by imaging the surface the template nucleic acids are immobilized on and analyzing the resulting image(s). The sequencing platform may be washed with a wash buffer to remove unincorporated nucleotides prior to signal detection. In some cases, prior to the next flow step (e.g., 1306), the label may be removed from the incorporated labeled T nucleotide (e.g., by cleaving the label from the nucleotide), before proceeding. Nucleotide flow, detection, and optionally cleavage, may be repeated according to a flow order that may or may not include repeating the flow cycle 1300 for any number of times. Flow step 1310 illustrates incorporation of two labeled A bases by the extending sequencing primer 1303 opposite the two T bases in the template strand, per the non-terminated nature of the flown nucleotides. The detected signal intensity indicating the incorporation of two A nucleotides may be greater than the signal intensity indicating the incorporation of one nucleotide. For simplicity, this Figure illustrates incorporation of two labeled A nucleotides in the same hybrid. However, flow-based sequencing may be performed on colonies of amplified molecules, e.g., each bead representing one colony, where an optically resolvable location contains multiple copies of the same template nucleic acid molecule (e.g., a location contains one amplified bead), such that the signal detected at an optically resolvable location represents an aggregate signal from the multiple copies of molecules. Thus, when using a nucleotide flow mixture containing labeled and unlabeled nucleotides of a same base type, the incorporation of the labeled nucleotides can be distributed across the multiple copies of the molecules, and the aggregate signal from the multiple copies detected. In some cases, for a majority of hybrids, at most a single labeled nucleotide may be incorporated into a single homopolymer stretch in a hybrid—the longer the homopolymer stretch, the more likely that more hybrids of the plurality of copies of hybrids in an optically resolvable location will incorporate one labeled nucleotide.

While each flow step in the example flow sequencing method in FIG. 13 results in incorporation of one or more nucleotides (and thus a detected signal indicating such incorporation), it should be appreciated that not all flow steps result in incorporation of nucleotides. In some flow steps, no nucleotide base may be incorporated (for example, in the absence of a complementary base in the template).

A nucleotide mixture that is provided during any one flow may comprise only labeled nucleotides, only unlabeled nucleotides, or a mixture of labeled and unlabeled nucleotides. The mixture of labeled and unlabeled nucleotides may be of any fraction of labeled nucleotides, such as at least or at most about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 99%. Labeled nucleotides may comprise a dye, fluorophore, or quantum dot, multiples thereof, and/or combination thereof. In some cases, nucleotides of different canonical base types may be labeled and detectable at a single frequency (e.g., using the same or different dyes). In other cases, nucleotides of different canonical base types may be labeled and detectable at different frequencies (e.g., using the same or different dyes). Labeled nucleotides may comprise an optical moiety (e.g., dye, fluorophore, quantum dot, label, etc.) coupled to a nucleobase via a linker, and the label from the labeled nucleotides may be removed by cleaving the linker to remove the optical moiety. Cleaving may comprise one or more stimuli, such as exposure to a chemical (e.g., reducing agent), an enzyme, light (e.g., UV light), or temperature change (e.g., heat).

Flow-based sequencing may comprise providing non-detected nucleotide flow(s), for example to skip sequencing of a region(s) of the template nucleic acid; to ensure completion of incorporation reactions across all template-primer hybrids in the reaction space; and/or phasing or re-phasing. A non-detected nucleotide flow may be referred to herein as a “dark flow”, “dark tap”, or “dark tap flow.” A detected nucleotide flow may be referred to herein as a “bright flow”, “bright tap”, or “bright tap flow.” Incorporation reactions may be incomplete in the reaction space when not all available incorporation sites in the template-primer hybrids have incorporated a complementary base, such as due to reaction kinetics and/or insufficient incubation time or reagents. In some cases, single-base flows of the same canonical base type may be provided consecutively (without intervening flow of a different nucleotide base type) for any number of consecutive flows, to ensure completion of incorporation reactions. A consecutive same-base flow may be referred to herein as a “double tap” or “double tap flow” if there are two consecutive flows, a “triple tap” or “triple tap flow” if there are three consecutive flows, or a “nth tap” or “nth tap flow” if there are n consecutive flows of the same base type. A double tap, triple tap, or nth tap flow may or may not be detected. Labels in a flow may or may not be removed (e.g., cleaved) prior to the double tap, triple tap, or nth tap flow. Detection of labeled nucleotides from a particular flow may be performed prior to, during, or subsequent to the double tap, triple tap, or nth tap flow. Accordingly, below are non-limiting examples of flow cycles that can be used in a larger flow order of flow-based sequencing methods, which may or may not be repeated and/or mixed and matched with other flow cycles, where * after a base represents a detected flow step, / between bases represents a mixed base flow, and a base without modification indicates an unlabeled base or a non-detected flow step:
Single-base flow cycle: e.g., [T*, A*, C*, G*]
Single-base flow cycle with double tap: e.g., [T*, T, A*, A, C*, C, G*, G]
Mixed base flow cycle, all labeled: e.g., [T*, A*/C*/G*]
Mixed base flow cycle, some unlabeled: e.g., [T*, A/C*/G]
Mixed base flow cycle, some unlabeled: e.g., [T, A*/C*/G*]
Skip region base flow cycle: e.g., [T/A/C or G/A/T]
Three base flow cycle: e.g., [T, A, C].

Additional sequencing schemes are described in U.S. Pat. Pub. Nos. 2021/0017593A1, 2022/0064728A1, and 2022/0154272A1, each of which is incorporated by reference herein in its entirety.

Sequencing with Capping

Sequencing methods may comprise contacting a nucleic acid molecule complex (or a sequencing primer-template nucleic acid complex) with a capping reagent. A sequencing primer is also referred to herein as an extending primer or growing nucleic acid strand. Any capping reagent described herein may be used. The capping reagent may be provided prior to, during, or subsequent to the nucleic acid molecule complex contacting a labeled reagent (e.g., a labeled nucleotide or other labeled substrate) to incorporate the labeled reagent. The capping reagent may be added with a labeled nucleotide, unlabeled nucleotide, a cleavage reagent, another reagent, light-input, energy-input, and/or any change in condition (e.g., for a scar immolation reaction). The capping reagent may be added subsequent to providing or adding a labeled nucleotide, unlabeled nucleotide, a cleavage reagent, another reagent, light-input, energy-input, and/or any change in condition (e.g., for a scar immolation reaction). The capping reagent may be added prior to providing or adding a labeled nucleotide, unlabeled nucleotide, a cleavage reagent, another reagent, light-input, energy-input, and/or any change in condition (e.g., for a scar immolation reaction).

The capping reagent may be provided with or prior to providing a subsequent nucleotide mixture to the nucleic acid molecule complex for incorporation. The capping reagent may be provided with or prior to detecting a label from an incorporated labeled reagent (e.g., labeled nucleotide).

Accordingly, a method for sequencing a nucleic acid molecule may comprise (a) incorporating a labeled nucleotide into a growing nucleic acid strand hybridized to the nucleic acid molecule, wherein the labeled nucleotide is coupled to a dye via a cleavable linker; (b) detecting a signal from the dye; (c) cleaving the cleavable linker and contacting the growing nucleic acid strand with a capping reagent, wherein the capping reagent is configured to generate a capped moiety on the growing nucleic acid strand. The method may further comprise determining or generating a sequencing read of the nucleic acid molecule based at least on the signal. The operations of (a) incorporation of a labeled nucleotide, (b) detecting a signal from the labeled nucleotide, if any, and (c) cleaving and contacting a capping reagent, may be repeated any number of times, with or without any intervening flows or operations as described elsewhere herein, to determine or generate the sequencing read of the nucleic acid molecule.

The nucleotides (e.g., labeled nucleotide) used in these methods may be terminated nucleotides. The nucleotides used in these methods may be non-terminated nucleotides. Nucleotides (e.g., labeled nucleotide) may be provided in a nucleotide flow comprising all labeled nucleotides, all unlabeled nucleotides, or a mixture of labeled and unlabeled nucleotides. A nucleotide flow may include nucleotides of a single canonical base type (e.g., A, T, G, C, U), or a mixture of canonical base types.

In some cases, the capping reagent may be provided to the growing nucleic acid strand in a mixture comprising an additional nucleotide. The additional nucleotide may be of a different canonical base type as the labeled nucleotide. Alternatively, the additional nucleotide may be of a same canonical base type as the labeled nucleotide. Beneficially, where the growing nucleic acid strand has failed to incorporate all available nucleotides in a first nucleotide flow comprising the labeled nucleotide (e.g., fail to incorporate all nucleotides of a same canonical base type in a homopolymer region of the template nucleic acid molecule, fail to incorporate any nucleotides) such as due to reaction kinetics, the subsequent, consecutive flow of additional nucleotides of the same canonical base type as nucleotides in the first nucleotide flow may complete the incorporation reaction for the growing nucleic acid strand before proceeding to interrogating/incorporating the growing nucleic acid strand with a nucleotide of a different base type. Not completing all available incorporation reactions may create sequencing phasing issues downstream where the nucleic acid molecule is being sequenced in synchrony with a colony of nucleic acid molecules. A subsequent nucleotide flow of a same canonical base type that is consecutively flowed (with no intervening different base type nucleotide flow) may be referred to herein as a “chase” flow.

The additional nucleotide may be an unlabeled nucleotide of the same canonical base type as the labeled nucleotide. The additional nucleotide may be a second labeled nucleotide of the same canonical base type as the labeled nucleotide. In such cases, the method may further comprise detecting a second signal from the second labeled nucleotide. The method may further comprise processing the signal and the second signal to determine or generate the sequencing read. For example, if the first nucleotide flow did not yield a signal (0 signal units) and the second nucleotide flow yielded a signal (1 signal units), the signals may be added or otherwise processed to determine that a number of nucleotides corresponding to a total of 1 signal units was incorporated. In another example, if the first nucleotide flow yielded a signal (4 signal units) and the second nucleotide flow yielded a signal (1 signal units), the signals may be added or otherwise processed to determine that a number of nucleotides corresponding to a total of 5 signal units was incorporated. In another example, if the first nucleotide flow yielded a signal (2 signal units) and the second nucleotide flow did not yield a signal (0 signal units), the signals may be added or otherwise processed to determine that a number of nucleotides corresponding to a total of 1 signal units was incorporated. As seen in this example, the plurality of signals from the initial flow and chase flow(s) may be added or otherwise processed to sequence a non-homopolymer region or homopolymer region of the template nucleic acid. Any number of chase flows may be provided to the growing nucleic acid strand, which may or may not include labeled or unlabeled nucleotides, such as 2, 3, 4, 5, 6, or more chase flows, to complete available incorporation reactions. Where a chase flow comprises labeled nucleotides, labels may be cleaved after detection. Capping reagents may be provided to address the chemical scars formed after cleavage of the labels. The capping reagents may be provided with or prior to a chase flow. The capping reagents may be provided with or subsequent to a cleavage flow (e.g., to cleave the dye). Chemical scars that otherwise are inhibitory towards subsequent incorporation reactions may be capped by the capping reagent to reduce such inhibitory effect. Such a method may increase the likelihood of complete extension across all molecules of a colony and/or across homopolymeric regions of a template nucleic acid. The capping reagent may remain stably bound to the scarred nucleotide through subsequent nucleotide additions and cleavage steps.

In some cases, a cleavage reagent may be provided independently of the additional nucleotide. For example, the cleavage reagent may be provided prior to providing the additional nucleotide.

Accordingly, a method for sequencing a nucleic acid molecule may comprise (a) contacting a first nucleotide solution to a growing nucleic acid strand hybridized to the nucleic acid molecule, wherein the first nucleotide solution comprises first labeled nucleotides; (b) detecting a signal from the growing nucleic acid strand indicative of incorporation of a labeled nucleotide of the first labeled nucleotides; (c) contacting with the growing nucleic acid strand (i) a cleavage reagent configured to cleave a label from the labeled nucleotide to the growing nucleic acid strand and (ii) a capping reagent configured to generate a capped moiety on the growing nucleic acid strand from a cleaved linker of the labeled nucleotide; (d) contacting a second nucleotide solution to the growing nucleic acid strand, wherein the second nucleotide solution comprises second labeled nucleotides, wherein the first nucleotide solution and the second nucleotide solution comprise nucleotides of the same canonical base type; and (e) detecting a second signal from the growing nucleic acid strand indicative of incorporation of a labeled nucleotide of the second labeled nucleotides.

### Sequencing for Increased Homopolymer Detection Accuracy

### Mixed-Reversibly Terminated Flow Sequencing

In some cases, for a bright step, the growing strand may be contacted with only non-terminated nucleotides—here, if the template has a homopolymer portion, the growing strand may incorporate multiple non-terminated nucleotides in a single step, and thus signals detected from incorporated labeled nucleotides may have to be further resolved to determine the length of the homopolymer. For example, relatively stronger signals may correspond to longer homopolymer length as they are indicative that more labeled nucleotides have been incorporated, and relatively weaker signals may correspond to lower homopolymer length as they are indicative that fewer labeled nucleotides have been incorporated. For example, detected signals may be algorithmically processed to distinguish a 2-mer from a 3-mer or a 4-mer from a 7-mer. However, homopolymer length determination accuracy from these signals may decrease as homopolymer lengths become longer and/or goes above a certain resolution threshold (e.g., 5-mer, 6-mer, 7-mer, 8-mer, 9-mer, 10-mer, 11-mer, 12-mer, 13-mer, 14-mer, 15-mer, 16-mer, 17-mer, 18-mer, 19-mer, 20-mer, 21-mer, etc.), such as due to increasing quenching effects of dye moieties on incorporated labeled nucleotides, optical resolution limitations for signal collection, and/or computing limitations. Alternatively or in addition, nucleotide incorporation may be impeded by the presence of scars in the growing strand (e.g., as a result of cleaving labels from incorporated nucleotides). This can inhibit sequencing, e.g., by increasing phasing, by pausing or stopping incorporation. The present systems, methods, compositions, and kits address at least the abovementioned limitations by improving the accuracy of sequencing reads by reading a homopolymer section of a template in multiple shorter segments and by reducing the impact of scarring. The methods described herein are applicable to either sequencing single molecules or sequencing colonies of amplified template molecules.

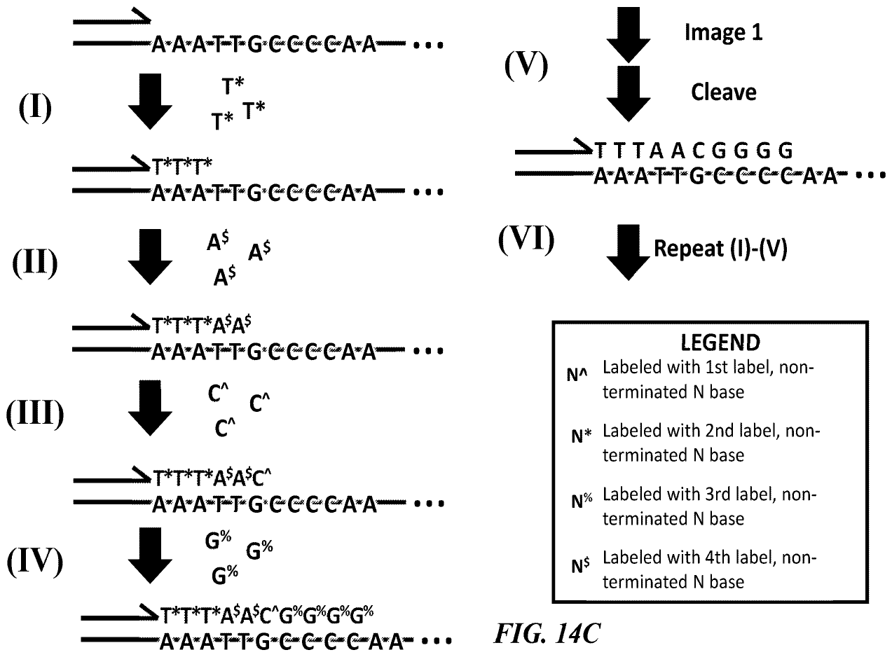

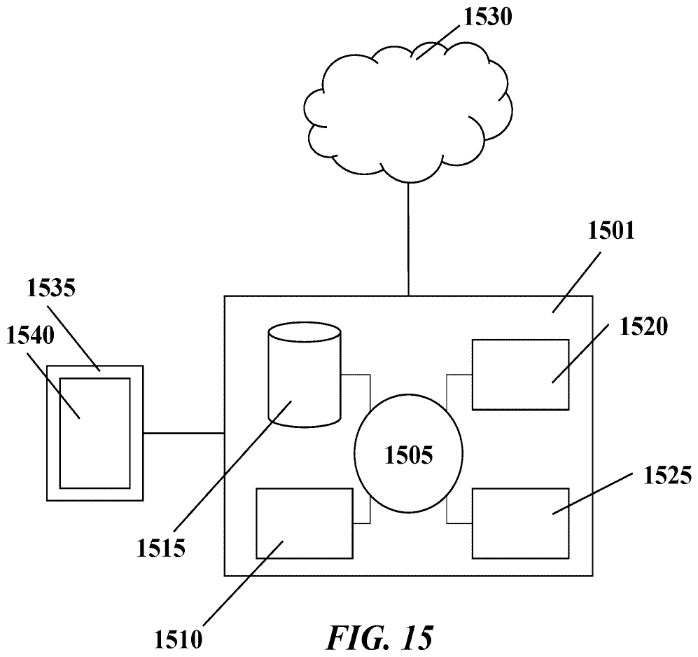

FIG. 14A illustrates an example of a mixed-reversibly terminated sequencing scheme. A template is hybridized to a growing strand which is ready to extend through a 6-mer polyA homopolymer portion in the template. In step (I), the first bright extension step, the growing strand is contacted with a nucleotide mixture comprising both labeled, non-terminated bases and reversibly terminated bases of T. The growing strand incorporates only two labeled, non-terminated T bases before incorporation is blocked by incorporation of a terminated T base, resulting in extending through 3 of 6 available T incorporation positions. In step (II), a first imaging is performed to collect first signals indicative of the first homopolymer segment, and then any labels and blocking moieties removed via cleaving. In step (III), the second bright extension step, step (I) is repeated where the growing strand is contacted with a nucleotide mixture comprising both labeled, non-terminated bases and reversibly terminated bases of T. This time, the growing strand incorporates only one labeled, non-terminated T base before incorporation is blocked by incorporation of a terminated T base, resulting in extending through 2 of 3 of the remaining available T incorporation positions. In step (IV), a second imaging is performed to collect second signals indicative of the second homopolymer segment, and then any labels and blocking moieties removed via cleaving. In step (V), in a dark extension step, the growing strand is contacted with unlabeled, non-terminated T bases to extend through all (in this case 1) of the remaining T incorporation positions. The data collected and/or determined from the two imaging actions (in steps (II) and (IV) respectively) may be processed (e.g., added) to determine a total homopolymer length of the homopolymer portion just sequenced. In this illustration, a determination of at least a 5-mer homopolymer length is made from the data collected. In step (VI), steps (I)-(V) may be repeated with a next, different canonical base type.

It will be appreciated that while this example includes only two bright extension steps ((I)-(II) and (III)-(IV)), any number of bright extension steps may be performed, which can increase the accuracy of the homopolymer length determination.

In some cases, for single molecule sequencing and colony-based sequencing, all non-terminated bases in a bright extension step may be labeled nucleotides. The terminated bases in a bright extension step may be labeled, unlabeled, or a mixture of both. In some cases, in dark extension steps (e.g., step (V)), the growing primer strand is contacted with labeled, non-terminated bases or a mixture of labeled and unlabeled non-terminated bases. This may be more efficient in terms of reagent storage space (e.g., obviating the need for separate reagent storage wells for different mixtures of unterminated bases for bright and dark extension steps). Dark extension steps do not include imaging.

In some cases, for colony-based sequencing, the non-terminated bases in a bright extension step may be a mixture of labeled and unlabeled nucleotides. The terminated bases in a bright extension step may be labeled, unlabeled, or a mixture of both. The mixture of labeled and unlabeled nucleotides in the non-terminated bases in the nucleotide reagent may be of any fraction of labeled nucleotides, such as at least or at most about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 99%. The mixture of labeled and unlabeled nucleotides in the terminated bases may be of any fraction of labeled nucleotides, such as at least or at most about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 99%. The mixture of labeled and unlabeled nucleotides in the nucleotide reagent may be of any fraction of labeled nucleotides, such as at least or at most about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 99%. Different fractions of labeled and unlabeled nucleotides, labeled and unlabeled nucleotides in the terminated bases, and/or labeled and unlabeled nucleotides in the non-terminated bases may be different for different base types (e.g., based on expected hmer lengths and/or quenching).

In some cases, for colony-based and single molecule sequencing, the nucleotide reagent can comprise a mixture of terminated and non-terminated nucleotides of any fraction of terminated to non-terminated nucleotides, such as or at most about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 99%. The fraction of terminated nucleotides will influence the average number of bases incorporated in each bright extension step. For example, if the fraction of terminated nucleotides is about 10%, then the average number of incorporated bases in each extending sequencing primer may be about 10 (e.g., 9 incorporated unterminated nucleotides and 1 incorporated terminated nucleotide). Similarly, if the fraction of terminated nucleotides is about 25%, then the average number of incorporated bases may be about 4 (e.g., 3 unterminated nucleotides and 1 terminated nucleotide). At most, one terminated base is expected to be incorporated in each bright extension step.

Any number of consecutive bright extension steps of a same canonical base type may be performed, such as 2, 3, 4, 5, 6, 7, 8 or more consecutive bright extension steps of a same canonical base type. In some cases, the respective number of consecutive bright steps may differ for different nucleotide base types (e.g., 2 consecutive bright steps for A and 3 consecutive bright steps for T). In some cases, a number of consecutive bright steps may be predetermined. In some cases, a number of bright steps may be determined based on relative signal brightness in images of a same nucleotide base type (e.g., Image 1 vs Image 2 in FIG. 14A

The sequencing method may comprise repeating the subjecting of a growing strand to a template to at least two consecutive bright extension steps followed by a dark extension step of the same canonical base type (e.g., A, G, C, T, U) with different bases for any number of times. For example, at least 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250, 300, 350, 400, 450, 500, 600, 700, 800, 900, 1000, 2000, 3000, 4000, 5000, 10,000 or more times.

A sequencing method may comprise subjecting a growing strand hybridized to a template to at least two consecutive bright extension steps followed by a dark extension step of the same canonical base type (e.g., A, G, C, T, U). For sequencing methods described herein, T and U are considered the same canonical base type. A bright extension step may comprise contacting the growing strand with a nucleotide mixture of both (1) labeled, non-terminated bases and (2) reversibly terminated bases of a same canonical base type. The reversibly terminated bases may be labeled or unlabeled, or a mixture of both. In some cases, the last bright extension step may comprise only non-terminated bases and omit the reversibly terminated bases. Beneficially, because of the fraction of reversibly terminated bases in the mixture, when at a long homopolymer stretch in the template, the growing strand is likely to incorporate a reversibly terminated base and block incorporation of the next base before fully extending through a long homopolymer stretch. This allows generating sequencing data by collecting signals (e.g., via imaging) from shorter homopolymer segment intervals, which results in a more accurate homopolymer base call for each segment. Sequencing data generated after each of the bright extension step(s) may be processed (e.g., signals added, images added, homopolymer lengths added, etc.) to determine length information of the homopolymer stretch. For example, a total length of the homopolymer may be determined with high accuracy. In another example, a minimum length of the homopolymer may be determined with high accuracy. Any labels may be removed from the growing strand between different bright extension steps, such as via cleavage, to allow for interval imaging and more efficient incorporation of the next succeeding base. Any blocking moieties may be removed from the growing strand between different extension steps (bright or dark), such as via cleavage, to allow incorporation of the next succeeding base in the next extension step. The bright extension steps may be followed by a dark extension step of the same canonical base type to (1) extend through any remaining portions of a homopolymer stretch that was not covered by the bright extension steps to prepare for interrogation with the next base type and/or (2) catch up any strands (e.g., with a colony) that were unable to incorporate a base(s), such as due to reaction kinetics.

### Mixed-Color Flow Sequencing, FRET

FIG. 14B illustrates an example of a mixed-color non-terminated sequencing scheme. A template is hybridized to a growing strand which is ready to extend through a 6-mer polyA homopolymer portion in the template. In step (I), the bright extension step, the growing strand is contacted with a nucleotide mixture comprising a first plurality of bases labeled with a first label and a second plurality of bases labeled with a second label, where all of the bases are T. The growing strand incorporates a mixture of Ts with the first and second labels (in this case only 5 Ts are incorporated; in some cases, 6 Ts may be incorporated). In step (II), a first imaging is performed to collect first signals indicative of the first label. In step (III), a second imaging is performed to collect second signals indicative of the second label, and then any labels are removed via cleaving. In step (IV), a dark extension is performed where the growing strand is contacted with unlabeled, non-terminated T bases to extend through all (in this case 1) of the remaining T incorporation positions. The data collected and/or determined from the two imaging actions (in steps (II) and (III) respectively) may be processed (e.g., added) to determine a total homopolymer length of the homopolymer portion just sequenced. In some cases, first or second signals may further be indicative of the second or first label, respectively. For example, in some cases, the first label may be a FRET donor, and the second label may be a FRET acceptor (or the reverse). In this illustration, a determination of at least a 5-mer homopolymer length is made from the data collected. In step (V), steps (I)-(IV) may be repeated with a next, different canonical base type. It will be appreciated that while this example includes only two bright extension steps ((I)-(II) and (III)-(IV)), any number of bright extension steps may be performed, which can increase the accuracy of the homopolymer length determination.

Beneficially, the use of at least two label types may improve homopolymer length determination. For instance, there may be less quenching between labels on incorporated nucleotides if there is a mixture of label types.

In some cases, for single molecule sequencing and colony-based sequencing, all non-terminated bases in a bright extension step may be labeled nucleotides. The terminated bases in a bright extension step may be labeled, unlabeled, or a mixture of both. In some cases, in dark extension steps (e.g., step (IV)), the growing primer strand is contacted with labeled, non-terminated bases or a mixture of labeled and unlabeled non-terminated bases. This may be more efficient in terms of reagent storage space (e.g., obviating the need for separate reagent storage wells for different mixtures of unterminated bases for bright and dark extension steps). Dark extension steps do not include imaging.

Here a method of sequencing is provided, comprising (a) contacting a growing strand hybridized to a template with a first reagent mixture comprising bases labeled with a first label type and bases labeled with a second label type, wherein the bases are of a first same canonical base type; (b) detecting a first signal indicative of incorporation of at least a subset of the bases labeled with the first label type in the growing strand, or lack thereof, to generate first sequencing data; (c) detecting a second signal indicative of incorporation of at least a subset of the bases labeled with the second label type in the growing strand, or lack thereof, to generate second sequencing data; and (d) processing the first sequencing data and the second sequencing data to determine length information of a homopolymer sequence in the template.

In some cases, the length information of the homopolymer sequence in the template comprises a minimum length of the homopolymer sequence. Alternatively, or in addition, the length information of the homopolymer sequence in the template comprises a total length of the homopolymer sequence.

In some case, the method may further comprise (e) contacting the growing strand with a second reagent mixture comprising unlabeled bases of the first canonical base type. The method may further comprise repeating (a)-(e) with a second canonical base type, a third canonical base type, and/or a fourth canonical base type. These steps may be repeated any number of time suitable for determining the sequence of a nucleic acid template molecule. For example, these steps may be repeated 1, 2, 3, 4, , 6, 7, 8, 9, 10, 20, 30, 40, 50, 60, 70, 80, 90, 100, 150, 200, 300, 400, 500, 600, 700, 800, 900, 1000, or more times.

In some cases, the first signal and the second signal may be localized to a single molecule of the template. Alternatively, the first signal and the second signal may be localized to a colony of molecules comprising the template.

In some cases, nucleotides are unterminated. In some cases, a mixture of terminated and unterminated nucleotides may be used.

In some cases, the template may be immobilized to a substrate surface. Alternatively or in addition, the template may be coupled to a bead that is immobilized to the substrate surface. Alternatively or in addition, the template may be coupled to a DNA nanoparticle (e.g., a DNA nanoball or DNA origami) that is immobilized to the substrate surface. In some cases, the substrate surface comprises at least 1,000,000 individually addressable locations and the template is immobilized to an individually addressable location in the at least 1,000,000 individually addressable locations.

### Multi-Color Flow Sequencing, Decreased Imaging

FIG. 14C illustrates an example of a mixed-color non-terminated sequencing scheme. A template is hybridized to a growing strand which is ready to extend through portion in the template. In step (I), a first bright extension step, the growing strand is contacted with a nucleotide mixture comprising non-terminated T bases labeled with a first label type. The growing strand incorporates three labeled, non-terminated T bases. In step (II), a second bright extension step, the growing strand is contacted with a nucleotide mixture comprising non-terminated A bases labeled with a second label type. The growing strand incorporates two labeled, non-terminated A bases. In step (III), a third bright extension step, the growing strand is contacted with a nucleotide mixture comprising non-terminated C bases labeled with a third label type. The growing strand incorporates one labeled, non-terminated C base. In step (IV), a fourth bright extension step, the growing strand is contacted with a nucleotide mixture comprising non-terminated G bases labeled with a fourth label type. The growing strand incorporates four labeled, non-terminated G bases. In step (V), imaging is performed to collect first, second, third, and fourth signals indicative of incorporation of T, A, C, and G, respectively. After imaging, any labels are removed via cleaving. In step (VI), steps (I)-(V) may be repeated.

It will be appreciated that one or more additional extension steps may be performed (e.g., an additional extension step for one or more base types). For instance, there may be a second extension step (e.g., comprising unlabeled, labeled, or a mixture of labeled and unlabeled Ts) performed after step (I) and prior to step (II). Similar additional extension steps may be performed for each nucleotide base type. In some cases, for single molecule sequencing and colony-based sequencing, all non-terminated bases in a bright extension step may be labeled nucleotides.

Beneficially, the method illustrated in FIG. 14C may permit the use of fewer imaging steps for sequencing. This may improve the speed of sequence (e.g., by replacing imaging steps for each extension step with an imaging step for every 2, 3, or 4 extension steps).

Here a method of sequencing a template is provided, comprising: (a) contacting a growing strand hybridized to the template with a first reaction mixture comprising nucleotides of a first canonical base type, wherein at least a portion of the nucleotides are labeled with a first label type; (b) contacting the growing strand hybridized to the template with a second reaction mixture comprising nucleotides of a second canonical base type, wherein at least a portion of the nucleotides are labeled with a second label type; and (c) detecting signal indicative of incorporation of nucleotides into the growing strand, or lack thereof, to generate first sequencing data.

The some cases, the method further comprises (d) contacting a growing strand hybridized to the template with a third reaction mixture comprising nucleotides of a third canonical base type, wherein at least a portion of the nucleotides are labeled with the first label type; E contacting the growing strand hybridized to the template with a fourth reaction mixture comprising nucleotides of a fourth canonical base type, wherein at least a portion of the nucleotides are labeled with the second label type; and (e) detecting signal indicative of incorporation of nucleotides into the growing strand, or lack thereof, to generate second sequencing data.

An additional method of sequencing a template is provided, comprising: (a) contacting a growing strand hybridized to the template with a first reaction mixture comprising nucleotides of one or more canonical base types, wherein at least a portion of nucleotides each canonical base type are labeled with a different respective label type; (b) contacting a growing strand hybridized to the template with a second reaction mixture comprising nucleotides of one or more canonical base types different from the canonical base types in the first reaction mixture, wherein at least a portion of nucleotides each canonical base type are labeled with a different respective label type; and (c) detecting signal indicative of incorporation of nucleotides into the growing strand, or lack thereof, to generate sequencing data.

An additional method of sequencing a template is provided, comprising: (a) contacting a growing strand hybridized to the template with a first reaction mixture comprising nucleotides of a first canonical base type, wherein at least a portion of the nucleotides are labeled with a first label type; (b) contacting the growing strand hybridized to the template with a second reaction mixture comprising nucleotides of a second canonical base type, wherein at least a portion of the nucleotides are labeled with a second label type; (c) contacting a growing strand hybridized to the template with a third reaction mixture comprising nucleotides of a third canonical base type, wherein at least a portion of the nucleotides are labeled with a third label type; (d) contacting the growing strand hybridized to the template with a fourth reaction mixture comprising nucleotides of a fourth canonical base type; and (e) detecting signal indicative of incorporation of nucleotides into the growing strand, or lack thereof, to generate first sequencing data.

In some cases, at least a portion of nucleotides in the fourth reaction mixture are labeled. In some cases, in each reaction mixture at least 1% of the nucleotides are labeled. Any percentage of the nucleotides in any reaction mixture may be labeled (with the remining percentage being unlabeled). In some case, in each reaction mixture 100% of the nucleotides are labeled.

In some cases, all of the label types are excited by the first illumination source. Alternatively, in some cases, each label type is excited by a separate illumination source. In some cases, at least two of the label types may be excited by the first illumination source. In some cases, the first and second label types are excited by a first illumination source, and the third and fourth label types are excited by a second illumination source. Any combination of labels may be excited by a first illumination source (e.g., 1, 2, 3, or 4 labels). For example, in some cases, all label types may be excited by the first illumination source.

In some cases, detection may be performed by one detector. In some cases, detection may be performed by two or more detectors. In some cases, detection may be performed by the same number of detectors as illumination sources. In some cases, detection may be performed by a different number of detectors from illumination sources (e.g., where a single illumination source excites multiple labels). In some cases, detection may be performed by a same number of detectors as labels. In some cases, detection may be performed by a different number of detectors from labels (e.g., where one detector is capable of simultaneously detecting and/or distinguishing multiple labels).

In some cases, in a single imaging step, signal (e.g., from 1, 2, 3, or 4 labels) may be localized to a single molecule of the template. Alternatively, in a single imaging step, signal (e.g., from 1, 2, 3, or 4 labels) may be localized to a colony of molecules comprising the template.

In some cases, nucleotides are unterminated. In some cases, a mixture of terminated and unterminated nucleotides may be used.

In some cases, the method further comprises, after detecting, cleaving any labels from incorporated nucleotides. In some cases, the method further comprises repeating the contacting, detecting, and cleaving, any number of times to determine a sequence of the template molecule. For instance, the steps may be repeated at least 1, 2, 3, 4, , 6, 7, 8, 9, 10, 20, 30, 40, 50, 60, 70, 80, 90, 100, 150, 200, 300, 400, 500, 600, 700, 800, 900, 1000, or more times to determine the sequence of the template.

In some cases, the template may be immobilized to a substrate surface. Alternatively or in addition, the template may be coupled to a bead that is immobilized to the substrate surface. Alternatively or in addition, the template may be coupled to a DNA nanoparticle (e.g., a DNA nanoball or DNA origami) that is immobilized to the substrate surface. In some cases, the substrate surface comprises at least 1,000,000 individually addressable locations and the template is immobilized to an individually addressable location in the at least 1,000,000 individually addressable locations.

In some cases, a combination of any of the systems, methods, and compositions described herein may be used.

### Computer Systems

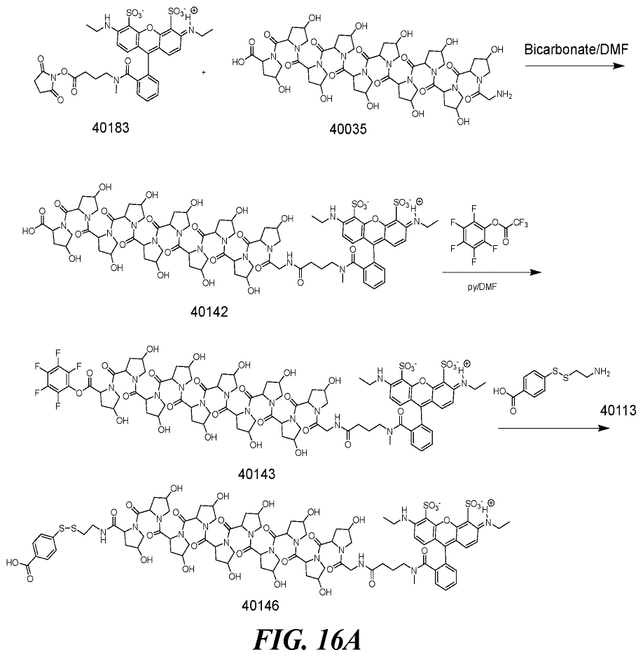

The present disclosure provides computer control systems that are programmed to implement methods of the disclosure. FIG. 15 shows a computer system 1501 that is programmed or otherwise configured to implement methods of the disclosure, such as to control the systems described herein (e.g., reagent dispensing, detecting, etc.) and collect, receive, and/or analyze sequencing information. The computer system 1501 can be an electronic device of a user or a computer system that is remotely located with respect to the electronic device. The electronic device can be a mobile electronic device.

The computer system 1501 includes a central processing unit (CPU, also “processor” and “computer processor” herein) 1505, which can be a single core or multi core processor, or a plurality of processors for parallel processing. The computer system 1501 also includes memory or memory location 1510 (e.g., random-access memory, read-only memory, flash memory), electronic storage unit 1515 (e.g., hard disk), communication interface 1520 (e.g., network adapter) for communicating with one or more other systems, and peripheral devices 1525, such as cache, other memory, data storage and/or electronic display adapters. The memory 1510, storage unit 1515, interface 1520 and peripheral devices 1525 are in communication with the CPU 1505 through a communication bus (solid lines), such as a motherboard. The storage unit 1515 can be a data storage unit (or data repository) for storing data. The computer system 1501 can be operatively coupled to a computer network (“network”) 1530 with the aid of the communication interface 1520. The network 1530 can be the Internet, an isolated or substantially isolated internet and/or extranet, or an intranet and/or extranet that is in communication with the Internet. The network 1530 in some cases is a telecommunication and/or data network. The network 1530 can include one or more computer servers, which can enable distributed computing, such as cloud computing. The network 1530, in some cases with the aid of the computer system 1501, can implement a peer-to-peer network, which may enable devices coupled to the computer system 1501 to behave as a client or a server. The CPU 1505 can execute a sequence of machine-readable instructions, which can be embodied in a program or software. The instructions may be stored in a memory location, such as the memory 1510. The instructions can be directed to the CPU 1505, which can subsequently program or otherwise configure the CPU 1505 to implement methods of the present disclosure. Examples of operations performed by the CPU 1505 can include fetch, decode, execute, and writeback. The CPU 1505 can be part of a circuit, such as an integrated circuit. One or more other components of the system 1501 can be included in the circuit. In some cases, the circuit is an application specific integrated circuit (ASIC).

The storage unit 1515 can store files, such as drivers, libraries and saved programs. The storage unit 1515 can store user data, e.g., user preferences and user programs. The computer system 1501 in some cases can include one or more additional data storage units that are external to the computer system 1501, such as located on a remote server that is in communication with the computer system 1501 through an intranet or the Internet.

The computer system 1501 can communicate with one or more remote computer systems through the network 1530. For instance, the computer system 1501 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1501 via the network 1530.

Methods as described herein can be implemented by way of machine (e.g., computer processor) executable code stored on an electronic storage location of the computer system 1501, such as, for example, on the memory 1510 or electronic storage unit 1515. The machine executable or machine-readable code can be provided in the form of software. During use, the code can be executed by the processor 1505. In some cases, the code can be retrieved from the storage unit 1515 and stored on the memory 1510 for ready access by the processor 1505. In some situations, the electronic storage unit 1515 can be precluded, and machine-executable instructions are stored on memory 1510. The code can be pre-compiled and configured for use with a machine having a processor adapted to execute the code or can be compiled during runtime. The code can be supplied in a programming language that can be selected to enable the code to execute in a pre-compiled or as-compiled fashion.

Aspects of the systems and methods provided herein, such as the computer system 1501, can be embodied in programming. Various aspects of the technology may be thought of as “products” or “articles of manufacture” typically in the form of machine (or processor) executable code and/or associated data that is carried on or embodied in a type of machine-readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine-readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, e.g., as shown in the drawings. Volatile storage media include dynamic memory, such as the main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1501 can include or be in communication with an electronic display 1535 that comprises a user interface (UI) 1540 for providing, for example, results of a nucleic acid sequence (e.g., sequence reads). Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1505. The algorithm can, for example, perform error correction on processed sequencing signals.

### Numbered Embodiments

The following embodiments recite non-limiting permutations of combinations of features disclosed herein. Other permutations of combinations of features are also contemplated. In particular, each of these numbered embodiments is contemplated as depending from or relating to every previous or subsequent numbered embodiment, independent of their order as listed.

Embodiment 1: A labeled reagent, comprising: a substrate; a linker, comprising a cleavable portion; a nucleic acid moiety, wherein the nucleic acid moiety is attached to the substrate via the linker; and one or more detectable moieties coupled to the nucleic acid moiety.

Embodiment 2: The labeled reagent of embodiment 1, wherein the substrate comprises a nucleotide base.

Embodiment 3: The labeled reagent of embodiment 1, wherein the substrate comprises a protein.

Embodiment 4: The labeled reagent of any one of embodiments 1-3, wherein the nucleic acid moiety comprises an oligonucleotide.

Embodiment 5: The labeled reagent of embodiment 4, wherein the oligonucleotide is double-stranded, comprising a first strand and a second strand.

Embodiment 6: The labeled reagent of embodiment 4 or embodiment 5, wherein the first strand of the oligonucleotide is coupled to the one or more detectable moieties.

Embodiment 7: The labeled reagent of embodiment 6, wherein the second strand of the oligonucleotide is not covalently coupled to the one or more detectable moieties.

Embodiment 8: The labeled reagent of any one of embodiments 5-7, wherein the first strand of the oligonucleotide comprises a sequence of at least a first and one or more additional canonical base types, wherein bases of the first canonical base type are coupled to detectable moieties.

Embodiment 9: The labeled reagent of embodiment 8, wherein the sequence of the first strand of the oligonucleotide comprises an alternation of the first canonical base type and the additional canonical base types, respectively.

Embodiment 10: The labeled reagent of embodiment 8, wherein the sequence of the first strand of the oligonucleotide comprises a series of nucleotide bases in the following order: one or more nucleotide bases of the additional canonical base types (Z); and a nucleotide base of the first canonical base type (X).

Embodiment 11: The labeled reagent of embodiment 8, wherein the sequence of the first strand of the oligonucleotide comprises a series of nucleotide bases of the first canonical base type (X) and the additional canonical base types (Z) in the form of (ZₙX)ᵢ, wherein: n is a number of bases of the additional canonical base types (Z), and n is an integer between 1 and 20; and i is a number of repeating units of a nucleotide base of the first canonical base type and n nucleotide bases of the additional canonical base types, and i is an integer between 1 and 10.

Embodiment 12: The labeled reagent of embodiment 8, wherein the first strand of the oligonucleotide comprises a sequence of at least three canonical base types.

Embodiment 13: The labeled reagent of embodiment 12, wherein the first strand of the oligonucleotide comprises a sequence of at least four canonical base types.

Embodiment 14: The labeled reagent of embodiment 12 or embodiment 13, wherein only a single canonical base type is coupled to detectable moieties of the one or more detectable moieties.

Embodiment 15: The labeled reagent of any one of embodiments 1-13, wherein the nucleic acid moiety comprises a predetermined two- or three-dimensional shape.

Embodiment 16: The labeled reagent of embodiment 15, wherein the predetermined two dimensional or three-dimensional shape encloses the one or more detectable moieties.

Embodiment 17: The labeled reagent of embodiment 15, wherein the predetermined two dimensional or three-dimensional shape further comprises one or more attachment sites for coupling to detectable moieties.

Embodiment 18: The labeled reagent of any one of embodiments 15-17, wherein the predetermined two dimensional or three dimensional shape comprises one or more single stranded nucleic acid molecules.

Embodiment 19: The labeled reagent of any one of embodiments 15-18, wherein the predetermined two dimensional or three dimensional shape comprises one or more double stranded or partially double stranded nucleic acid molecules.

Embodiment 20: The labeled reagent of any one of embodiments 1-19, wherein the one or more detectable moieties coupled to the nucleic acid moiety comprise fluorescent dyes.

Embodiment 21: The labeled reagent of embodiment 20, wherein the fluorescent dyes comprise ATTO 390, ATTO 425, ATTO 465, ATTO 488, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 643, ATTO 647, ATTO 655, ATTO Oxa12, ATTO 665, ATTO 680, ATTO 700, ATTO 725, ATTO 740, UG 524.2, UG 524.1, KK1119, KK9046, Abberior STAR635, Abberior STAR635P, or Kam.

Embodiment 22: The labeled reagent of any one of embodiments 1-19, wherein the one or more detectable moieties coupled to the nucleic acid moiety comprise one or more fluorescent nanoparticles.

Embodiment 23: The labeled reagent of embodiment 22, wherein the one or more fluorescent nanoparticles are selected from the set consisting of Q-dots, fluorescent beads, gel particles, or a combination thereof.

Embodiment 24: A method for sequencing, comprising: providing a primer-hybridized template nucleic acid molecule; and contacting the primer-hybridized template nucleic acid molecule with nucleotides, wherein at least a subset of the nucleotides comprises a labeled reagent according to embodiments 1-23.

Embodiment 25: The method of embodiment 24, further comprising (c) detecting one or more signals from the primer-hybridized template nucleic acid molecule.

Embodiment 26: The method of embodiment 24, wherein the nucleotides are of a first canonical base type.

Embodiment 27: A method of pre-enrichment, comprising: contacting a template nucleic acid to a support, wherein the support comprises a DNA nanostructure, the DNA nanostructure comprising (i) a pre-enrichment site configured to bind to the template nucleic acid and (ii) an amplification site configured to bind an amplified derivative of the template nucleic acid, to generate a support-template complex, wherein the DNA nanostructure comprises fewer pre-enrichment sites than amplification sites, wherein the template nucleic acid is capable of hybridizing to the pre-enrichment site and not to the amplification site.

Embodiment 28: The method of embodiment 27, wherein the pre-enrichment site comprises a first oligonucleotide molecule comprising (1) a first sequence comprising a capture sequence complementary to an adapter sequence of the template nucleic acid and (2) a second sequence comprising an amplification primer sequence, and wherein the amplification site comprises a second oligonucleotide molecule comprising the second sequence, wherein the amplification site does not comprise the first sequence.

Embodiment 29: The method of embodiment 28, wherein the template nucleic acid is hybridized to the first sequence, and further comprising extending (1) the first oligonucleotide molecule to generate a first extended molecule and (2) the template nucleic acid to generate a second extended molecule.

Embodiment 30: The method of embodiment 29, wherein the second extended molecule is removed from the first extended molecule, and further comprising attaching the second extended molecule or a derivative of the second extended molecule to the second oligonucleotide molecule.

Embodiment 31: The method of any one of embodiments 27-30, wherein the DNA nanostructure comprises a plurality of amplification sites.

Embodiment 32: The method of any one of embodiments 27-31, wherein the DNA nanostructure comprises at most 1% pre-enrichment sites from all attachment sites including pre-enrichment sites and amplification sites on the DNA nanostructures.

Embodiment 33: The method of any one of embodiments 27-32, wherein the DNA nanostructure is bound to at most one template nucleic acid.

Embodiment 34: The method of any one of embodiments 27-33, wherein the DNA nanostructure further comprises a surface attachment site configured to attach to a binder of a substrate.

Embodiment 35: The method of any one of embodiments 27-34, further comprising contacting a plurality of template nucleic acids, including the template nucleic acid, and a plurality of supports, including the support, to generate a plurality of support-template complexes wherein a majority of the plurality of support-template complexes comprises a single template nucleic acid of the plurality of template nucleic acids.

Embodiment 36: The method of embodiment 35, wherein the plurality of template nucleic acids is provided at lower concentration than the plurality of supports.

Embodiment 37: The method of nay one of embodiments 27-36, further comprising providing a diffusion-limiting agent with the support and the template nucleic acid.

Embodiment 38: The method of embodiment 37, wherein the diffusion-limiting agent comprises polyethylene glycol (PEG).

Embodiment 39: The method of any one of embodiments 27-38, further comprising constructing the DNA nanostructure using a scaffold strand and a plurality of staple strands.

Embodiment 40: The method of any one of embodiments 27-39, wherein the DNA nanostructure comprises a cross-link.

Embodiment 41: The method of any one of embodiments 27-40, wherein the DNA nanostructure comprises a dideoxy NTP (ddNTP).

Embodiment 42: The method of any one of embodiments 27-41, further comprising loading the support-template complex onto a substrate.

Embodiment 43: A composition, comprising: a support, wherein the support comprises a DNA nanostructure, the DNA nanostructure comprising (i) a pre-enrichment site configured to bind to a template nucleic acid and (ii) an amplification site configured to bind an amplified derivative of the template nucleic acid, wherein the DNA nanostructure comprises fewer pre-enrichment sites than amplification sites, wherein the template nucleic acid is capable of hybridizing to the pre-enrichment site and not to the amplification site.

Embodiment 44: The composition of embodiment 43, wherein the pre-enrichment site comprises a first oligonucleotide molecule comprising (1) a first sequence comprising a capture sequence complementary to an adapter sequence of the template nucleic acid and (2) a second sequence comprising an amplification primer sequence, and wherein the amplification site comprises a second oligonucleotide molecule comprising the second sequence, wherein the amplification site does not comprise the first sequence.

Embodiment 45: The composition of any one of embodiments 43-44, further comprising a plurality of supports, each support of the plurality of supports comprising a DNA nanostructure.

Embodiment 46: The composition of any one of embodiments 43-45, further comprising the template nucleic acid.

Embodiment 47: The composition of embodiment 46, wherein the template nucleic acid is not bound to the support.

Embodiment 48: The composition of embodiment 46, wherein the template nucleic acid is bound to the support.

Embodiment 49: The composition of any one of embodiments 43-48, wherein the DNA nanostructure further comprise a surface attachment site.

Embodiment 50: The composition of any one of embodiments 43-49, further comprising a substrate.

Embodiment 51: The composition of any one of embodiments 43-50, further comprising a diffusion-limiting agent.

Embodiment 52: The composition of embodiment 51, wherein the diffusion-limiting agent comprises polyethylene glycol (PEG).

Embodiment 53: The composition of any one of embodiments 43-52, wherein the DNA nanostructure comprises a cross-link.

Embodiment 54: The composition of any one of embodiments 43-53, wherein the DNA nanostructure comprises a dideoxy NTP (ddNTP).

Embodiment 55: A kit, comprising: a support, wherein the support comprises a DNA nanostructure, the DNA nanostructure comprising (i) a pre-enrichment site configured to bind to a template nucleic acid and (ii) an amplification site configured to bind an amplified derivative of the template nucleic acid, wherein the DNA nanostructure comprises fewer pre-enrichment sites than amplification sites, wherein the template nucleic acid is capable of hybridizing to the pre-enrichment site and not to the amplification site.

Embodiment 56: The kit of embodiment 55, wherein the pre-enrichment site comprises a first oligonucleotide molecule comprising (1) a first sequence comprising a capture sequence complementary to an adapter sequence of the template nucleic acid and (2) a second sequence comprising an amplification primer sequence, and wherein the amplification site comprises a second oligonucleotide molecule comprising the second sequence, wherein the amplification site does not comprise the first sequence.

Embodiment 57: The kit of any one of embodiments 55-56, further comprising a plurality of supports, each support of the plurality of supports comprising a DNA nanostructure.

Embodiment 58: The kit of any one of embodiments 55-57, further comprising the template nucleic acid.

Embodiment 59: The kit of embodiment 58, wherein the template nucleic acid is not bound to the support.

Embodiment 60: The kit of embodiment 58, wherein the template nucleic acid is bound to the support.

Embodiment 61: The kit of any one of embodiments 55-60, wherein the DNA nanostructure further comprise a surface attachment site.

Embodiment 62: The kit of any one of embodiments 55-61, further comprising a substrate.

Embodiment 63: The kit of any one of embodiments 55-62, further comprising a diffusion-limiting agent.

Embodiment 64: The kit of embodiment 63, wherein the diffusion-limiting agent comprises polyethylene glycol (PEG).

Embodiment 65: The kit of any one of embodiments 55-64, wherein the DNA nanostructure comprises a cross-link.

Embodiment 66: The kit of any one of embodiments 55-65, wherein the DNA nanostructure comprises a dideoxy NTP (ddNTP).

Embodiment 67: A method, comprising contacting a plurality of supports to a substrate to immobilize the plurality of supports to the substrate, wherein the plurality of supports comprises a plurality of DNA nanostructures, wherein the plurality of DNA nanostructures comprises amplification sites.

Embodiment 68: A method, comprising contacting a plurality of supports to a substrate to immobilize the plurality of supports to the substrate, wherein the plurality of supports comprises a plurality of DNA nanostructures, wherein the plurality of DNA nanostructures comprises amplification sites, pre-enrichment sites, surface sites, nanostructure connection sites, or a combination thereof.

Embodiment 69: A method for sequencing a nucleic acid molecule, comprising: (a) contacting a first nucleotide solution to a growing nucleic acid strand hybridized to the nucleic acid molecule, wherein the first nucleotide solution comprises first labeled nucleotides; (b) detecting a signal from the growing nucleic acid strand indicative of incorporation of a labeled nucleotide of the first labeled nucleotides; (c) contacting with the growing nucleic acid strand (i) a cleavage reagent configured to cleave a label from the labeled nucleotide to the growing nucleic acid strand and (ii) a capping reagent configured to generate a capped moiety on the growing nucleic acid strand from a cleaved linker of the labeled nucleotide; (d) contacting a second nucleotide solution to the growing nucleic acid strand, wherein the second nucleotide solution comprises second labeled nucleotides, wherein the first nucleotide solution and the second nucleotide solution comprise nucleotides of the same canonical base type; and (e) detecting a second signal from the growing nucleic acid strand indicative of incorporation of a labeled nucleotide of the second labeled nucleotides.

Embodiment 70: The method of embodiment 69, further comprising using the first signal and the second signal to determine a sequencing read of the nucleic acid molecule.

Embodiment 71: The method of any one of embodiments 69-70, wherein the capping reagent comprises a disulfide group.

Embodiment 72: The method of embodiment 71, wherein the capping reagent comprises dipyridyl disulfide (DPDS) or pyridyl ethyl amine disulfide (PEAD).

Embodiment 73: The method of any one of embodiments 69-72, wherein the first labeled nucleotides are non-terminated nucleotides.

Embodiment 74: The method of any one of embodiments 69-73, wherein the first labeled nucleotides and the second labeled nucleotides comprise a single canonical base type.

Embodiment 75: The method of any one of embodiments 69-74, wherein the capping reagent is provided to the growing nucleic acid strand in a mixture with the second nucleotide solution.

Embodiment 76: The method of any one of embodiments 69-75, wherein the first nucleotide solution comprises a mixture of labeled and unlabeled nucleotides.

Embodiment 77: The method of any one of embodiments 69-76, wherein the nucleic acid molecule is immobilized to a substrate.

Embodiment 78: The method of embodiment 77, wherein the nucleic acid molecule is coupled to a bead immobilized to the substrate.

Embodiment 79: The method of embodiment 78, wherein the bead comprises a plurality of nucleic acid molecules, including the nucleic acid molecule, comprising an identical sequence, wherein the plurality of nucleic acid molecules are hybridized to a plurality of growing nucleic acid strands, including the growing nucleic acid strand.

Embodiment 80: The method of any one of embodiments 69-79, wherein in (c), cleaving of the label from the labeled nucleotide by the cleavage reagent generates a thiol scar on the growing nucleic acid strand.

Embodiment 81: The method of any one of embodiments 69-80, wherein the cleavage reagent is selected from the group consisting of: tris(3-hydroxypropyl) phosphine (THP), β-mercaptoethanol (β-ME), dithiothreitol (DTT), tris(2-carboxyethyl)phosphine (TCEP), Ellman's reagent, hydroxylamine, glutathione, and cyanoborohydride.

Embodiment 82: The method of any one of embodiments 69-81, wherein the labeled nucleotide of the first labeled nucleotides comprises a cleavable linker, wherein the cleavable linker comprises a disulfide bond.

Embodiment 83: The method of any one of embodiments 69-82, wherein the labeled nucleotide of the first labeled nucleotides comprises a hydroxyproline linker.

Embodiment 84: The method of any one of embodiments 69-82, wherein the first labeled nucleotides and the second labeled nucleotides comprise a same type of dye.

Embodiment 85: A method for sequencing a nucleic acid molecule, comprising: incorporating a labeled nucleotide into a growing nucleic acid strand hybridized to a nucleic acid molecule, wherein the labeled nucleotide is coupled to a dye via a cleavable linker; detecting a signal from the dye; cleaving the cleavable linker; and contacting the growing nucleic acid strand with a capping reagent, wherein the capping reagent comprises pyridyl ethyl amine disulfide.

Embodiment 86: A kit for sequencing, comprising: a plurality of labeled nucleotides comprising a cleavable linker; and a capping reagent comprising pyridyl ethyl amine disulfide.

Embodiment 87: The kit of embodiment 86, further comprising a cleavage reagent.

Embodiment 88: The kit of embodiment 87, wherein the cleavage reagent is selected from the group consisting of tris(3-hydroxypropyl) phosphine (THP), β-mercaptoethanol (β-ME), dithiothreitol (DTT), tris(2-carboxyethyl)phosphine (TCEP), Ellman's reagent, hydroxylamine, glutathione, and cyanoborohydride.

Embodiment 89: A method, comprising: (a) contacting a growing strand hybridized to a template with a first reagent mixture comprising labeled, non-terminated bases and reversibly terminated bases of a first same canonical base type and detecting a first signal indicative of incorporation of at least a subset of the labeled, non-terminated bases of the first reagent mixture in the growing strand, or lack thereof, to generate first sequencing data; (b) reversing termination of the reversibly terminated bases of the first reagent mixture incorporated in the growing strand, if any; (c) contacting the growing strand with a second reagent mixture comprising labeled, non-terminated bases and terminated bases of the first same canonical base type and detecting a second signal indicative of incorporation of at least a subset of the labeled, non-terminated bases of the second reagent mixture in the growing strand, or lack thereof, to generate second sequencing data; and (d) processing the first sequencing data and the second sequencing data to determine length information of a homopolymer sequence in the template

Embodiment 90: The method of embodiment 89, wherein the length information of the homopolymer sequence in the template comprises a minimum length of the homopolymer sequence.

Embodiment 91: The method of any of embodiments 89 or 90, wherein the length information of the homopolymer sequence in the template comprises a total length of the homopolymer sequence.

Embodiment 92: The method of any of embodiments 89-91, further comprising (e) reversing termination of the reversibly terminated bases of the second reagent mixture incorporated in the growing strand, if any, and (f) contacting the growing strand with a third reagent mixture comprising unlabeled, non-terminated bases of the first same canonical base type.

Embodiment 93: The method of embodiment 92, further comprising (g) repeating (a)-(f) with a second same canonical base type different from the first canonical base type.

Embodiment 94: The method of embodiment 93, further comprising (h) repeating (a)-(f) with a third same canonical base type different from the first canonical base type and the second canonical base type.

Embodiment 95: The method of embodiment 94, further comprising (i) repeating (a)-(f) with a fourth same canonical base type different from the first canonical base type, the second canonical base type, and the third canonical base type.

Embodiment 96: The method of embodiment 95, further comprising (j) repeating (a)-(i) at least 10 times.

Embodiment 97: The method of any of embodiments 89-96, wherein the first signal is localized to a single molecule of the template.

Embodiment 98: The method of any of embodiments 89-96, wherein the first signal is localized to a colony of molecules comprising the template.

Embodiment 99: The method of any of embodiments 89-98, wherein the template is immobilized to a substrate surface.

Embodiment 100: The method of embodiment 99, wherein the template is coupled to a bead that is immobilized to the substrate surface.

Embodiment 101: The method of embodiment 99, wherein the template is coupled to a DNA nanoparticle that is immobilized to the substrate surface.

Embodiment 102: The method of embodiment 101, wherein the DNA nanoparticle comprises a DNA nanoball.

Embodiment 103: The method of embodiment 101, wherein the DNA nanoparticle comprises DNA origami.

Embodiment 104: The method of any of embodiments 99-103, wherein the substrate surface comprises at least 1,000,000 individually addressable locations and the template is immobilized to an individually addressable location in the at least 1,000,000 individually addressable locations.

Embodiment 105: A method of sequencing a template, comprising: (a) contacting a growing strand hybridized to the template with a first reaction mixture comprising nucleotides of a first canonical base type, wherein at least a portion of the nucleotides are labeled with a first label type; (b) contacting the growing strand hybridized to the template with a second reaction mixture comprising nucleotides of a second canonical base type, wherein at least a portion of the nucleotides are labeled with a second label type; and (c) detecting signal indicative of incorporation of nucleotides into the growing strand, or lack thereof, to generate first sequencing data.

Embodiment 106: The method of embodiment 104, further comprising: (d) contacting a growing strand hybridized to the template with a third reaction mixture comprising nucleotides of a third canonical base type, wherein at least a portion of the nucleotides are labeled with the first label type; (e) contacting the growing strand hybridized to the template with a fourth reaction mixture comprising nucleotides of a fourth canonical base type, wherein at least a portion of the nucleotides are labeled with the second label type; and (f) detecting signal indicative of incorporation of nucleotides into the growing strand, or lack thereof, to generate second sequencing data.

Embodiment 107: The method of embodiment 106, further comprising combining first sequencing data and second sequencing data.

Embodiment 108: A method of sequencing a template, comprising: (a) contacting a growing strand hybridized to the template with a first reaction mixture comprising nucleotides of one or more canonical base types, wherein at least a portion of nucleotides each canonical base type are labeled with a different respective label type; (b) contacting a growing strand hybridized to the template with a second reaction mixture comprising nucleotides of one or more canonical base types different from the canonical base types in the first reaction mixture, wherein at least a portion of nucleotides each canonical base type are labeled with a different respective label type; and (c) detecting signal indicative of incorporation of nucleotides into the growing strand, or lack thereof, to generate sequencing data

Embodiment 109: A method of sequencing a template, comprising: (a) contacting a growing strand hybridized to the template with a first reaction mixture comprising nucleotides of a first canonical base type, wherein at least a portion of the nucleotides are labeled with a first label type; (b) contacting the growing strand hybridized to the template with a second reaction mixture comprising nucleotides of a second canonical base type, wherein at least a portion of the nucleotides are labeled with a second label type; (c) contacting a growing strand hybridized to the template with a third reaction mixture comprising nucleotides of a third canonical base type, wherein at least a portion of the nucleotides are labeled with a third label type; (d) contacting the growing strand hybridized to the template with a fourth reaction mixture comprising nucleotides of a fourth canonical base type, wherein at least a portion of the nucleotides are labeled with a fourth label type; and (e) detecting signal indicative of incorporation of nucleotides into the growing strand, or lack thereof, to generate first sequencing data.

Embodiment 110: The method of any one of embodiments 104-109, wherein, in each reaction mixture at least 1% of the nucleotides are labeled.

Embodiment 111: The method of any one of embodiments 104-110, wherein in each reaction mixture 100% of the nucleotides are labeled.

Embodiment 112: The method of any one of embodiments 104-111, wherein the first and second label types are excited by a first illumination source, and the third and fourth label types are excited by a second illumination source.

Embodiment 113: The method of any of embodiments 104-112, wherein signal is localized to a single molecule of the template.

Embodiment 114: The method of any of embodiments 104-112, wherein signal is localized to a colony of molecules comprising the template.

Embodiment 115: The method of any of embodiments 104-114, wherein the template is immobilized to a substrate surface.

Embodiment 116: The method of embodiment 115, wherein the template is coupled to a bead that is immobilized to the substrate surface.

Embodiment 117: The method of embodiment 115, wherein the template is coupled to a DNA nanoparticle that is immobilized to the substrate surface.

Embodiment 118: The method of embodiment 117, wherein the DNA nanoparticle comprises a DNA nanoball.

Embodiment 119: The method of embodiment 117, wherein the DNA nanoparticle comprises DNA origami.

Embodiment 120: The method of any of embodiments 115-119, wherein the substrate surface comprises at least 1,000,000 individually addressable locations and the template is immobilized to an individually addressable location in the at least 1,000,000 individually addressable locations.

Embodiment 121: A method of sequencing, comprising: (a) contacting a growing strand hybridized to a template with a first reaction mixture comprising nucleotides of a first canonical base type, wherein at least a portion of the nucleotides are labeled with a first label type; (b) contacting the growing strand hybridized to the template with a second reaction mixture comprising nucleotides of a second canonical base type, wherein at least a portion of the nucleotides are labeled with a second label type; (c) contacting a growing strand hybridized to the template with a third reaction mixture comprising nucleotides of a third canonical base type, wherein at least a portion of the nucleotides are labeled with a third label type; (d) contacting the growing strand hybridized to the template with a fourth reaction mixture comprising nucleotides of a fourth canonical base type; and (e) detecting signal indicative of incorporation of nucleotides into the growing strand, or lack thereof, to generate first sequencing data.

Embodiment 122: The method of embodiment 121, wherein in the fourth reaction mixture at least a portion of the nucleotides are labeled with a fourth label type.

Embodiment 123: The method of any one of embodiments 121-122, wherein, in each reaction mixture at least 1% of the nucleotides are labeled.

Embodiment 124: The method of any one of embodiments 121-123, wherein in each reaction mixture 100% of the nucleotides are labeled.

Embodiment 125: The method of embodiment 121, wherein in the fourth reaction mixture the nucleotides are unlabeled.

Embodiment 126: The method of any one of embodiments 104-125, wherein at least two of the label types are excited by the first illumination source.

Embodiment 127: The method of any one of embodiments 104-125, where all of the label types are excited by the first illumination source.

Embodiment 128: The method of any one of embodiments 104-125, wherein each label type is excited by a separate illumination source.

Embodiment 129: The method of any one of embodiments 104-128, wherein the detection is performed by one detector.

Embodiment 130: The method of any one of embodiments 104-128, wherein the detection is performed by one or more detectors.

Embodiment 131: The method of any one of embodiments 104-130, wherein the nucleotides are unterminated.

Embodiment 132: The method of any one of embodiments 104-131, further comprising, after detecting, cleaving any labels from incorporated nucleotides.

Embodiment 133: The method of embodiment 132, further comprising repeating the contacting, detecting, and cleaving, at least 10 times to determine the sequence of the template.

Embodiment 134: A method of sequencing, comprising (a) contacting a growing strand hybridized to a template with a first reagent mixture comprising bases labeled with a first label type and bases labeled with a second label type, wherein the bases are of a first same canonical base type; (b) detecting a first signal indicative of incorporation of at least a subset of the bases labeled with the first label type in the growing strand, or lack thereof, to generate first sequencing data; (c) detecting a second signal indicative of incorporation of at least a subset of the bases labeled with the second label type in the growing strand, or lack thereof, to generate second sequencing data; and (d) processing the first sequencing data and the second sequencing data to determine length information of a homopolymer sequence in the template

Embodiment 135: The method of embodiment 134, wherein the length information of the homopolymer sequence in the template comprises a minimum length of the homopolymer sequence.

Embodiment 136: The method of any of embodiments 134 or 135, wherein the length information of the homopolymer sequence in the template comprises a total length of the homopolymer sequence.

Embodiment 137: The method of any one of embodiments 134-136 further comprising (e) contacting the growing strand with a second reagent mixture comprising unlabeled bases of the first canonical base type.

Embodiment 138: The method of embodiment 137, further comprising (f) repeating (a)-(e) with a second same canonical base type different from the first canonical base type.

Embodiment 139: The method of embodiment 138, further comprising (g) repeating (a)-(e) with a third same canonical base type different from the first canonical base type and the second canonical base type.

Embodiment 140: The method of embodiment 139, further comprising (h) repeating (a)-(e) with a fourth same canonical base type different from the first canonical base type, the second canonical base type, and the third canonical base type.

Embodiment 141: The method of embodiment 140, further comprising (i) repeating (a)-(h) at least 10 times.

Embodiment 142: The method of any of embodiments 134-141, wherein the first signal and the second signal are localized to a single molecule of the template.

Embodiment 143: The method of any of embodiments 134-141, wherein the first signal and the second signal are localized to a colony of molecules comprising the template.

Embodiment 144: The method of any of embodiments 134-143, wherein the template is immobilized to a substrate surface.

Embodiment 145: The method of embodiment 144, wherein the template is coupled to a bead that is immobilized to the substrate surface.

Embodiment 146: The method of embodiment 144, wherein the template is coupled to a DNA nanoparticle that is immobilized to the substrate surface.

Embodiment 147: The method of embodiment 146, wherein the DNA nanoparticle comprises a DNA nanoball.

Embodiment 148: The method of embodiment 146, wherein the DNA nanoparticle comprises DNA origami.

Embodiment 149: The method of any of embodiments 144-148, wherein the substrate surface comprises at least 1,000,000 individually addressable locations and the template is immobilized to an individually addressable location in the at least 1,000,000 individually addressable location.

### EXAMPLES

These examples are provided for illustrative purposes only and are not intended to limit the scope of the claims provided herein.

### Example 1: General Synthetic Principles

Certain of the following examples illustrate various methods of making linkers and labeled reagents described herein. It is understood that one skilled in the art may be able to make these compounds by similar methods or by combining other methods known to one skilled in the art. It is also understood that one skilled in the art would be able to make other compounds in a similar manner as described below by using the appropriate starting materials and modifying synthetic routes as needed. In general, starting materials and reagents can be obtained from commercial vendors or synthesized according to sources known to those skilled in the art or prepared as described herein.

Unless otherwise noted, reagents and solvents used in synthetic methods described herein are obtained from commercial suppliers. Anhydrous solvents and oven-dried glassware may be used for synthetic transformations sensitive to moisture and/or oxygen. Yields may not be optimized. Reaction times may be approximate and may not be optimized. Materials and instrumentation used in synthetic procedures may be substituted with appropriate alternatives. Column chromatography and thin layer chromatography (TLC) may be performed on reverse-phase silica gel unless otherwise noted. Nuclear magnetic resonance (NMR) and mass spectra may be obtained to characterize reaction products and/or monitor reaction progress.

### Example 2: Synthesizing Hypn

A large order hydroxyproline moiety, Hypn (e.g., n>=20, 30, 40, 50, etc.), as used and described herein, may be synthesized by adding two or more smaller order hydroxyproline moieties, Hypn (e.g., n>=20, 15, 10, 9, 8, 7, 15, 5, 4, 3, etc.). For example, a Hyp30 is created by adding a Hyp10 and Hyp20. In another example, a Hyp40 is created by adding two Hyp20's. In another example, a Hyp12 is created by adding two Hyp15's. As seen from these examples, the two or more smaller order Hypn moieties may or may not be the same lengths.

### Example 3: Preparation of Dye-Labeled Nucleotides

A set of dye-labeled nucleotides designed for excitation at about 530 nm is prepared. Excitation at 530 nm may be achieved using a green laser, which may be readily available, high-powered, and stable. There are many commercially available fluorescent dyes with excitation at or near 530 nm that are inexpensive and have a variety of properties (hydrophobic, hydrophilic, positively charged, negatively charged). Synthetic routes to such dyes may be shorter and cheaper than those for longer wavelength dyes. Moreover, certain green dyes may have significantly less self-quenching than red dyes, potentially allowing for the use of higher labeling fractions (e.g., as described herein).

A viable reagent set that may be used for a sequencing application consists of each of four canonical nucleotides or analogs thereof with cleavable green dyes. An optimal set may be prepared by varying each component of a labeled nucleotide structure to obtain an array of candidate labeled nucleotides with varying properties. The resultant nucleotides are evaluated (e.g., as described below), and certain labeled nucleotides are optimized for concentration and labeling fraction (e.g., the ratio of labeled to unlabeled nucleotide in a flow).

A synthetic method for preparing G*-B-H (see FIG. 15) is shown in FIGS. 15A and 15B. Similar methods may be used to prepare other labeled nucleotides. As the components used include amino acids, there are multiple routes to the final product. Synthetic considerations include the tendency for hydrolysis of the triphosphate (to the diphosphate and monophosphates) under heat or acidic conditions, the tendency for disulfide to decompose in the presence of triethylamine and ammonia, preventing the use of acid-labile protecting groups, and preventing the use of trifluoroacetamide or FMOC protecting groups.

Preparation of PN 40142. A solution of ATTO 532 succinimidyl ester (ATTO-TEC, PN 40183; 5 mg=4.15 mol) in 100 μL of DMF was mixed with gly-hyp-hyp-hyp-hyp-hyp-hyp-hyp-hyp-hyp-hyp (custom synthesis from Genscript, PN 40035; 8.5 mg=7 mol) in 170 μL 0.1 M bicarbonate in a 1.5 mL Eppendorf tube. The reaction was purified on a Phenomenex reverse phase C18 semi-prep column (Gemini 5 μM C18, 250×10 mm) using a 10%->40% acetonitrile vs. 0.1 M triethylammonium acetate gradient over 115 minutes. The fractions containing product 40142 were combined and concentrated to dryness. The yield was determined by diluting a fraction and measuring the optical density (OD) at 1533 nm, using an extinction coefficient for the dye of 130,000 cm⁻¹M⁻¹. The yield was 50%. The structure was confirmed by mass spectrometry in negative ion mode: m/z calculated for C₈₁H₁₀₃N₁₄O₃₁S₂⁻, 1831.15; found: 1831.8.

Preparation of PN 40143. PN 40142 (4 mol) was suspended in 100 μL DMF in a 1.5 mL eppendorf tube. Pyridine (20 μL) and pentafluorophenyl trifluoroacetate (20 μL) were added to the DMF solution, which was heated to 50° C. for five minutes. A portion (1 μL) of the reaction mixture was precipitated into 0.4% HCl; the aqueous solution remains colorless, indicating complete conversion to the active pentafluorophenyl ester. The remainder of the reaction was precipitated into the dilute acidic solution and the aqueous solution pipetted off. The residue was washed with hexane and dried to a highly colored solid (PN 40143).

Preparation of PN 401415. PN 40143 was dissolved in 100 μL DMF and mixed with disulfide PN 40113 (5 mg, 20 mol) in DMF. Diisopropylethylamine (5 μL) was added to the mixture. The mixture was purified on reverse phase HPLC using a 20%→50% acetonitrile vs. 0.1 M TEAA gradient over 115 minutes. Two dye-colored fractions were obtained at 8.8 min and 9.5 min. The fraction at 9.5 min was identified by mass spectrometry to be the desired product: m/z calculated for C₉₀H₁₁₁N₁₅O₃₂S₄²⁻, [M-H]²⁻, 1020.84; found: 1021.1.

Preparation of PN 40147. PN 401415 was suspended in 100 μL DMF in a 1.5 mL eppendorf tube. Pyridine (20 μL) and pentafluorophenyl trifluoroacetate (20 μL) were added to the DMF solution and heated to 50° C. for five minutes. A portion (1 μL) of the reaction mixture was precipitated into 0.4% HCl; the aqueous solution remained colorless, indicating complete conversion to the active, pentafluorophenyl ester. The remainder of the reaction was precipitated into the dilute acidic solution and the aqueous solution pipetted off. The residue was washed with hexane and dried to a highly colored solid (PN 40147).

Preparation of PN 40150. PN 40147 was dissolved in 50 μL DMF in a 1.5 mL eppendorf tube. A solution of 0.5 μmol 7-deaza-7-propargylamino-2′-deoxyguanosine-5′-triphosphate in 50 μL 1 M bicarbonate was prepared and added to the tube. After remaining overnight at 4° C., the product was purified on HPLC; the fraction at 12 min, purified using a 20%→50% acetonitrile vs. 0.1 M TEAA gradient over 115 minutes, contained the desired product: m/z calculated for C₁₀₄H₁₂₉N₂₀O₄₄P₃S₄²⁻, [M-H]²⁻, 1291.33; found: 1292.4.

### Example 4: Effect of Scars from Cleaved Labels on Preceding Bases on Subsequent Misincorporations

To test the effect of different chemical scars on preceding bases on subsequent misincorporation events, three different nucleotide mixtures were assayed against a same Pacific blue-labeled template. Chemical scars on preceding bases are generally formed from cleaving labels from previously labeled nucleotides and optionally treating such scars. The template used in the assays had a long stretch of CT repeats (total 20 bases) followed by a GA sequence. The three different nucleotide mixtures include: (1) a first mixture, comprising an unlabeled dGTP/dATP mix, (2) a second mixture, comprising an unlabeled dGTP-PA/dATP-PA mix, where -PA represents a propargylamine (PA) scar, and (3) a third mixture, comprising an unlabeled dATP-PEAD/dGTP-PEAD mix, where -PEAD represents a pyridyl ethyl amine disulfide (PEAD)-capped scar. Each nucleotide mixture further comprised either a mix of dCTP-Atto532 and dUTP-Atto532 or just dUTP-Atto532. When both labeled U and C were present, rapid changes in the FRET signals were observed, suggesting rapid incorporation of all unlabeled nucleotides (through the CT repeat section in the template), followed by signals from the labeled U and C (incorporated in the GA sequence). However, when only the labeled U was present, in the absence of a labeled C, misincorporation of the labeled U opposite the G (of the GA sequence) in the template was observed—without misincorporation, the template cannot be extended beyond the CT repeat section in the template. The rates of misincorporation observed were as follows: second mixture (-PA mix)>first mixture (unlabeled)>>third mixture (-PEAD mix). Thus, it can be concluded that the presence of the PEAD-capped scar on a preceding base is significantly inhibitory for a following misincorporation event, whereas a PA scar actually accelerates such misincorporation event compared to the wild-type nucleotides. In a follow-on experiment, mixtures with various combinations of the PA and PEAD-capped scars on the two nucleotides (dATP, dGTP) were assayed to confirm that the type of scar on the base immediately preceding the misincorporation site most strongly affects the rate of misincorporation, with a PEAD-capped scar being significantly more inhibitory than the PA scar.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

1.-100. (canceled)

101. A method for sequencing nucleic acid molecules, comprising:
(a) loading a plurality of particles on a surface of a substrate to self-assemble and immobilize to the surface at a plurality of individually addressable locations,
wherein the particles comprise beads, deoxyribonucleic acid (DNA) nanoballs, DNA nanostructures, nanoparticles, or a combination thereof,
wherein the plurality of individually addressable locations is spaced apart on the surface by at least a diameter of a particle of the plurality of particles,
wherein each of the plurality of particles comprises a moiety configured to attach to a template nucleic acid molecule of a plurality of template nucleic acid molecules,
wherein subsequent to immobilizing the plurality of particles, the plurality of template nucleic acid molecules are immobilized to the plurality of individually addressable locations, wherein a majority of the plurality of individually addressable locations comprises at most one template nucleic acid molecule of the plurality of template nucleic acid molecules attached thereto; and
(b) sequencing the plurality of template nucleic acid molecules, or reverse complements thereof, immobilized at the plurality of individually addressable locations on the surface of the substrate.

102. The method of claim 101, wherein the plurality of particles is cleaved or removed from the surface prior to sequencing the plurality of template nucleic acid molecules in (b).

103. The method of claim 101, wherein the plurality of template nucleic acid molecules is attached to the plurality of particles during the sequencing in (b).

104. The method of claim 101, wherein the plurality of particles is attached to the surface, and wherein the plurality of template nucleic acid molecules is coupled to the plurality of particles.

105. The method of claim 104, wherein the plurality of particles is attached to the surface via a click chemistry reaction, complementary hybridization, specific binding, non-specific binding, electrostatic interaction, or crosslinking between the plurality of particles, or moieties thereof, and the surface, or moieties thereof.

106. The method of claim 101, wherein the plurality of template nucleic acid molecules is attached to the surface, and wherein the plurality of particles is coupled to the plurality of template nucleic acid molecules.

107. The method of claim 106, wherein the plurality of template nucleic acid molecules is attached to the surface via a click chemistry reaction, complementary hybridization, specific binding, non-specific binding, electrostatic interaction, or crosslinking between the plurality of template nucleic acid molecules, or moieties thereof, and the surface, or moieties thereof.

108. The method of claim 101, wherein (a) comprises (i) loading to the surface the plurality of particles, and (ii) subsequently attaching a plurality of nucleic acid molecules to respective attachment sites on the plurality of particles to provide or generate the plurality of template nucleic acid molecules immobilized at the plurality of individually addressable locations.

109. The method of claim 101, wherein (a) comprises (i) attaching a plurality of nucleic acid molecules to respective attachment sites on the plurality of particles, and (ii) subsequently loading to the surface the plurality of particles attached to the plurality of nucleic acid molecules to provide or generate the plurality of template nucleic acid molecules immobilized at the plurality of individually addressable locations.

110. The method of claim 101, wherein the plurality of particles comprises a plurality of beads, wherein a bead of the plurality of beads comprises one or more primers, wherein (i) a primer of the one or more primers is extended to generate a template nucleic acid molecule of the plurality of template nucleic acid molecules or (ii) a template nucleic acid molecule of the plurality of template nucleic acid molecules is hybridized to a primer of the one or more primers.

111. The method of claim 101, wherein the plurality of particles comprises a plurality of DNA nanostructures.

112. The method of claim 111, wherein (a) comprises contacting the plurality of template nucleic acid molecules with the plurality of particles in presence of a diffusion-limiting or crowding agent.

113. The method of claim 112, wherein the diffusion-limiting or crowding agent comprises polyethylene glycol (PEG).

114. The method of claim 101, wherein the plurality of particles comprises a plurality of DNA nanoballs.

115. The method of claim 114, wherein the plurality of DNA nanoballs is coupled to the plurality of template nucleic acid molecules.

116. The method of claim 114, wherein the plurality of DNA nanoballs comprises the plurality of template nucleic acid molecules.

117. The method of claim 101, wherein the plurality of particles is subjected to self-assemble on a surface with (i) a diameter of at least 150 millimeters (mm) or (ii) a first lateral dimension of at least 100 mm and a second lateral dimension of at least 100 mm.

118. The method of claim 101, wherein the substrate is unpatterned, untextured, or both prior to loading the plurality of particles in (a).

119. The method of claim 101, wherein prior to loading the plurality of particles in (a), the substrate is patterned with DNA nanostructures.

120. The method of claim 101, wherein each of the plurality of particles comprises a single moiety configured to attach to a template nucleic acid molecule of the plurality of template nucleic acid molecules.
